(19) United States
(12) Patent Application Publication
(10) Pub. No.: US 2026/0207408 A1
(43) Pub. Date: Jul. 23, 2026

(54) SYSTEMS AND METHODS FOR SURGICAL POSITIONING

(71) Applicant: Mizuho Orthopedic Systems, Inc., Union CIty, CA (US)

(72) Inventors: Stephan Schmid, San Francisco, CA (US); Vincent Hodges, San Jose, CA (US); Mark DeSilets, San Jose, CA (US); Peter Le, Union City, CA (US)

(21) Appl. No.: 19/370,739

(22) Filed: Oct. 27, 2025

Related U.S. Application Data
(60) Provisional application No. 63/712,518, filed on Oct. 27, 2024.
(60) Provisional application No. 63/769,429, filed on Mar. 10, 2025.

(51) Int. Cl. A61G 13/00; A61B 46/20; A61B 46/27; A61G 13/08; A61G 13/10; A61G 13/12

(52) U.S. Cl. CPC A61G 13/0063; A61B 46/27; A61G 13/08; A61G 13/129; A61B 2046/201; A61G 13/105; A61G 13/1245; A61G 13/125

(57) ABSTRACT

A positioning system for a surgical procedure is provided. The positioning system may include a surgery table, a spar base, and a spar extending from the spar base to support at least one leg of a patient. The spar base may be positioned beneath a platform of the surgery table and may translate a distance along the surgery table.

### RELATED APPLICATION DATA

This application claims priority to U.S. Provisional Application No. 63/712,518, filed Oct. 27, 2024 and U.S. Provisional Application No. 63/769,429, filed Mar. 10, 2025, the disclosures of which are hereby incorporated by reference in their entireties.

### FIELD

This invention relates to medical procedures, and more particularly, to surgery tables used for multiple types of surgeries.

### BACKGROUND

Patient support apparatuses assist in positioning and supporting a patient before, during, and after a medical procedure. Surgery tables are patient support apparatuses used in surgical procedures, and allow a surgeon access to a surgical site and the ability to maneuver, position, or reposition the patient before, during, and after the surgical procedure. Surgeons endeavor to adjust surgery tables while minimizing or preventing risk to the patient and by maintaining a sterile barrier between the surgical site and sources of contamination, e.g., surfaces of the patient's body, which may host pathogens that are harmful if they enter the patient through an incision, and surgical equipment, which may not be completely free of contaminants even after being sanitized. Surgical drapes may be used to create the sterile barrier by being draped over at least a portion of the patient and the surgery table.

### SUMMARY

In one general aspect, system may include a surgery table having a distal end and a proximal end, and a platform configured to support at least a portion of the patient. System may also include a spar base positioned beneath the platform at the distal end. System may furthermore include a spar extending from the spar base and configured to support at least one leg of the patient. System may in addition include where the spar base is configured to translate a distance along the surgery table between the distal end and the proximal end. Other embodiments of this aspect include corresponding computer systems, apparatus, and computer programs recorded on one or more computer storage devices, each configured to perform the actions of the methods.

Implementations may include one or more of the following features.

System where the spar base may include a spar mount configured to receive the spar, the spar mount having a receptacle having an inclined wall. System where the spar may include a projection, the spar mount configured to receive the projection. System where the receptacle may include a retractable electrical connection, and where the spar may include an electrical connection configured to couple to the electrical connection of the receptacle. System where the spar base may include one or more joints configured to articulate the spar mount. System where the one or more joints may include metal. System where the spar base having a distal end, and where the spar extends from the distal end of the spar base. System where the spar base may include a pivot post, and where the one or more joints may include a first joint configured to articulate the spar mount in a pitch direction, the first joint rotatably coupled to the pivot post. System where the pivot post extends from the distal end of the spar base. System where the one or more joints may include a second joint configured to articulate the spar mount in a yaw direction, the second joint rotatably coupled to the pivot post. System where a first rotational axis for the first joint and a second rotational axis for the second joint intersect. System where the spar base may include a lift assist mechanism configured to provide lift assist for the spar mount to articulate the spar mount in the pitch direction. System where the lift assist mechanism may include a slider coupled to the spar mount and configured to translate along the pivot post. System where the slider is coupled to the spar mount by one or more linkages. System where the lift assist mechanism may include a ball nut and a screw, the slider being coupled to the ball nut configured to translate along the screw. System where the lift assist mechanism may include a first spring coupled to the ball nut, the first spring configured to provide lift assist for the spar to articulate the spar in a downward pitch direction. System where the first spring is configured to be compressed to allow the spar mount to articulate the spar in the downward pitch direction. System where the first spring is a coil spring coupled to the screw. System where the lift assist mechanism may include a gearbox and one or more second springs, each second spring having a mainspring coupled to the screw by the gearbox, the one or more second springs configured to provide lift assist for the spar to articulate the spar in an upward pitch direction. System where the one or more second springs is compressed to provide lift assist for the spar to articulate the spar in the upward pitch direction. System where the one or more second springs may include a plurality of springs stacked on top of one another to provide torque for the lift assist mechanism. System where the spar may include essentially of carbon fiber, and where the spar base may include metal. System where the surgery table may include a top-down lower body imaging window extending from a distal edge of the surgery table, and where the spar base is not may include within the imaging window. System where the surgery table further may include a traction mechanism, and where the spar base is configured to translate along the surgery table such that a traction load is applied by the traction mechanism to the at least one leg of the patient. System where the platform may include a lead screw configured to translate the spar base, and configured to apply traction by pushing the spar outwardly from the surgery table in an inferior direction. System where the lead screw is coupled to the spar base. System where the surgery table further may include a strain gauge configured to measure a traction load. System where the surgery table further may include a display configured to indicate a measurement of the traction load. System where the spar base is configured to articulate the spar in a pitch direction, where the spar is configured to articulate in a downward pitch direction to a downward pitch angle from an axis extending along the spar when the spar is in a straight configuration, the downward pitch angle being up to about 45 degrees. System where the spar may include a distal end and a proximal end, and where the distal end of the spar does not may include a traction mechanism. System where the distal end of the spar may include a foot support module configured to support at least one foot of the at least one leg of the patient, and where the distal end of the spar is configured to reach a position proximate a ground surface supporting the system when the spar is articulated in a downward pitch direction such that the foot of the patient is proximate the ground surface. System where the distal end of the spar further may include a handle configured to allow a user to articulate the spar, the handle having one or more buttons to unlock the spar to allow the spar to be articulated. System where the spar may include a distal end and a proximal end, and where a distal end of the spar may include one or more first traction controls configured to adjust the traction load. System where the surgery table further may include one or more second traction controls configured to adjust the traction load. System where the second traction controls are disposed along the platform proximate the distal end of the surgery table. System where the second traction controls may include a first button configured to incrementally translate the spar in an inferior direction. System where the second traction controls may include a second button configured to incrementally translate the spar in a superior direction. System where the second traction controls may include a third button configured to allow for manual translation of the spar. System where the second traction controls may include a fourth button configured to rotate a foot support configured to support a foot of the patient. System may include a pendant, where the surgery table further may include one or more third traction controls configured to adjust the traction load, and where the pendant may include the third traction controls. System where the surgery table may include a top-down lower body imaging window, and where the spar is laterally offset from the lower body imaging window. System where the spar is not may include within the imaging window. System where the spar is at least partially may include within the imaging window. System where the surgery table may include a lateral edge, where the spar base is disposed along the lateral edge such that the spar extends from the lateral edge, and where the leg of the patient extends from the table interior to the spar. System where an axis extends along the spar when the spar is in a straight configuration, and where a leg axis along the leg of the patient is interior to the axis. System may include: a foot support module configured to support at least one foot of the at least one leg of the patient, where the foot support module is coupled to the spar. System where the foot support module extends outwardly from the spar in a medial direction such that the spar is medially offset from at least a portion of the leg of the patient. System where the surgery table further may include a pelvic port at the distal end of the surgery table, the pelvic port is configured to receive a plurality of attachments configured to support at least a portion of the patient. System where the pelvic port may include an electrical coupling. System may include a pelvic port adapter, where the pelvic port may include a rail, and where the pelvic port adapter may include a track, the track configured to receive the rail such that the pelvic port adapter is coupled to the pelvic port. System where the pelvic port adapter is configured to receive the plurality of attachments. System where the pelvic port adapter may include a protrusion. System where each of the plurality of attachments may include a receptacle configured to receive the protrusion of the pelvic port adapter. System where the spar may include a portion that is substantially radiolucent. System where the surgery table may include a lower body imaging window, and where the portion of the spar is at least partially may include within the imaging window. System where the spar may include a cross-section having a substantially hexagonal shape. System where the spar may include essentially of carbon fiber. System where the surgery table may include a lower body imaging window, and where at least a portion of the spar is may include within the imaging window. System where the spar may include a proximal end and a distal end, the distal end being distal to the proximal end, and where distal end of the spar is length-adjustable. System where the spar may include a proximal end and a distal end, the distal end being distal to the proximal end, and where the distal end is coupled to the proximal end by a center joint. System where the center joint is configured to raise or lower the distal end such that the distal end is at an angle of about 70 degrees relative to the proximal end. System may include: a manual lock proximate the center joint, where the center joint is configured to be locked via the manual lock. System where the manual lock is configured to be in a sterile space. System where the center joint is configured to be released via one or more joint controls remote from the center joint. System where the distal end of the spar may include a handle configured to articulate the spar, the handle having the one or more joint controls. Foot support mechanism where the foot support may include a user interface paddle configured to release the foot support from the foot support module. System where the proximal end may include a gear configured to control the orientation of the proximal end. System where the proximal end of the spar may include one or more links and a passive link, and where the center joint is coupled to the passive link that is coupled to the gear via one or more links. System where the one or more links may include a first link and a second link, and where the gear is configured to control the orientation of the first link. System may include a rotating link, where the second link is coupled to the rotating link, and where the rotating link, the passive link, the first link, and the second link form a 4-bar linkage configured to rotate around the passive link. System where the proximal end may include a locking mechanism configured to lock the orientation of the proximal end. System where one or more links may include a first link and a second link, and where the locking mechanism is configured to lock the first link. System where the locking mechanism may include a pair of gears configured to alternately rotate and lock the first link. System where the locking mechanism may include a proximal rotational shaft, a distal rotational shaft coupled to the first link, and a pair of gears having a proximal gear coupled to the proximal rotational shaft and a distal gear coupled to the distal rotational shaft. System where the proximal gear and the distal gear are coupled to one another such that the distal rotational shaft is configured to rotate relative to the proximal rotational shaft and locking the proximal rotational shaft locks the distal rotational shaft to lock the first link. System where the locking mechanism further may include a slider-crank mechanism configured to control the orientation of the proximal end. System where the slider-crank mechanism may include an electromagnetic brake, a ball screw, a ball nut coupled to the ball screw, and a slider link coupled to the ball nut and the proximal rotational shaft, where the ball screw is configured to rotate such that the ball nut translates vertically when the electromagnetic brake is unlocked, and where the ball screw is configured to stop rotating when the electromagnetic brake is locked such that the slider link is locked in position to lock the proximal rotational shaft to lock the first link. System where the slider-crank mechanism may include an electromagnetic brake and a slider link coupled to the proximal rotational shaft, where the slider link is configured to rotate the proximal rotational shaft when the electromagnetic brake is unlocked, and where the slider link is locked in position when the electromagnetic brake is locked such that the proximal rotational shaft is locked to lock the first link. System may include: a pocket configured to receive a pendant for controlling at least articulation of the spar. System where the pocket is disposable. System where the pocket may include an adhesive. System where the pocket is configured to be selectively attachable to a drape for the system along at least one of the surgery table and the spar. System may include: one or more universal attachment interface (UAI) ports configured to receive two or more table modules, each of the table modules being distinct from another one of the table modules. System where each of the one or more UAI ports may include an electrical connection. System where each of the table modules may include one or more UAI plugs, and where each of the one or more UAI ports is configured to receive the one or more UAI plugs. System where each of the table modules may include an UAI lock configured to retain the UAI plug in the UAI port. System where each of the UAI plugs may include a dog-bone shape. System where each of the UAI plugs may include first portion, a second portion, and a third portion between the first portion and the second portion, where the first portion and the second portion are substantially spherical, and where the third portion is substantially rectangular. System where the first portion and the second portion are configured to guide the UAI plug of the respective table module such that the UAI port receives the table module. System where the first portion and the second portion each may include a bevel shape configured to guide the UAI plug of the table module such that the UAI port receives the table module. Foot support mechanism may include one or more magnets to actuate the mechanical coupling, the one or more magnets are disposed in the sterile space. Foot support mechanism where the one or more magnets is configured to generate a magnetic field that extends to the working space. System where the UAI port may include a dog-bone shape. System where the UAI port may include first opening, a second opening, and a third opening between the first opening and the second opening, where the first opening and the second opening are substantially spherical, and where the third opening is substantially rectangular. System where the first opening and the second opening are configured to guide the UAI plug of the table module such that the UAI port receives the table module. System where the first opening and the second opening each may include a bevel shape configured to guide the UAI plug of the table module such that the UAI port receives the table module. Foot support mechanism where the gripping mechanism is configured to rotate inward when actuated to grip the foot support, and where the foot support is configured to rotate outward when released to release the foot support. System where the distal end of the spar may include at least one UAI port of the one or more UAI ports. System where the at least one UAI port of the distal end of the spar is configured to receive a foot support module, the two or more table modules having the foot support module. Foot support mechanism where the spar may include a proximal end, a center joint, and a distal end coupled to the proximal end of the spar by the center joint, where, as the gripping mechanism is actuated, the distal end of the spar is configured to fold substantially simultaneously as the proximal end of the spar translates relative to the surgery table when the center joint is actuated. Foot support mechanism where the rod may include mu metal configured to alternately magnetize in the presence of a magnetic field and essentially demagnetize in the absence of a magnetic field, and where the magnetic field is configured to actuate the gripping mechanism such that the rod is gripped and in the absence of a magnetic field is configured to release the gripping mechanism such that the rod is released. Foot support mechanism where the foot support may include a rod having the transverse axis and configured to be received by the gripping mechanism. System where the spar base may include at least one UAI port of the one or more UAI ports. System where the at least one UAI port of the distal end of the surgery table is configured to receive a femur hook module, the two or more table modules having the femur hook module. Foot support mechanism where the foot support may include one or more springs and a user interface paddle having a first position in which the one or more magnets does not generate the magnetic field and a second position in which the one or more magnets generates the magnetic field, the foot support being biased to the first position by the one or more springs. System where the at least one UAI port of the distal end of the surgery table is configured to receive a robotic arm module, the two or more table modules having the robotic arm module. Foot support mechanism where, in the second position, the one or more magnets contact a metal sidewall having a mu metal conduit having mu metal, the mu metal conduit coupled to the rod having mu metal such that the magnetic field generated by the one or more magnets is extended to the rod via the mu metal conduit. Foot support mechanism where the foot support module may include a magnetic sensor configured to actuate the gripping mechanism when the magnetic field is present. Foot support mechanism where the user interface paddle, in the second position, is configured to fold the distal end of the spar and translate the proximal end of the substantially simultaneously. Foot support mechanism where the foot support may include one or more magnets configured to generate the magnetic field. System where the distal end of the surgery table may include at least one UAI port of the one or more UAI ports. System where the at least one UAI port of the distal end of the surgery table is configured to receive a femur hook module, the two or more table modules having the femur hook module. System where the at least one UAI port of the distal end of the surgery table is configured to receive a robotic arm module, the two or more table modules having the robotic arm module. System may include: an UAI offset arm configured to couple to the one or more UAI ports, and where the one or more UAI ports is further configured to receive the UAI offset arm, the UAI offset arm configured to couple to the one or more UAI ports, and where the UAI offset arm is selectively disposed on the surgery table at any of the one or more UAI ports. System where the UAI offset arm may include an UAI plug configured to be received by each of the one or more UAI ports. System where the UAI offset arm may include an arm having a proximal end coupled to the UAI plug of the UAI offset arm and a distal end having at least one UAI port of the one or more UAI ports. System may include: a portable UAI, where the portable UAI is selectively coupled to at least one of the spar and the surgery table. System where the portable UAI is not configured to couple to the one or more UAI ports. System where the portable UAI may include at least one UAI port of the one or more UAI ports. Foot support mechanism where the foot support module is in a working space. Foot support mechanism where the foot support is in a sterile space. Foot support mechanism where a spar configured to support the at least one leg of the patient may include the foot support module. System may include: a trunk pad configured to be disposed along the platform, the trunk pad configured to support at least a torso of the patient. System where the trunk pad may include a center pad configured to be disposed under the torso. System where the center pad may include an opening and an insert, and where the center pad is configured to receive the insert, the insert configured to support at least a portion of the patient. System where the trunk pad may include one or more lateral pads configured to extend from the center pad in a lateral direction, the one or more lateral pads configured to support at least a portion of the patient. System where the trunk pad may include one or more hammock attachments, where each of the one or more lateral pads is coupled to the center pad via one of the one or more hammock attachments, and where each of the one or more hammock attachments may include a length of flexible material to adjust the extension of the respective lateral pad relative to the center pad. System may include: one or more lateral flaps configured to extend from the platform in the lateral direction. System where each of the one or more lateral flaps is configured to receive one of the lateral pads. System where each of the one or more lateral flaps is translatable such that the position of each of the one or more lateral flaps relative to the platform is adjustable in at least one of a lateral direction and a medial direction. System where each of the one or more lateral flaps is rotatable such that the pitch of the one or more lateral flaps relative to the platform is adjustable. System where each of the one or more lateral flaps is translatable and rotatable via a single control. System where each of the one or more lateral flaps is rotatable such that the pitch of the one or more lateral flaps relative to the platform is adjustable. System where each of the one or more lateral flaps is rotatable such that the position of each of the one or more lateral flaps relative to the platform is adjustable in at least one of an anterior direction and a posterior direction. System may include: one or more lateral flaps configured to extend from the platform in the lateral direction. System may include: a trunk pad configured to be disposed along the platform, the trunk pad configured to support at least a torso of the patient. System where the trunk pad may include a center pad configured to be disposed under the torso. System where the trunk pad may include one or more lateral pads configured to extend from the center pad in a lateral direction, the one or more lateral pads configured to support at least a portion of the patient. System where the trunk pad may include one or more hammock attachments, where each of the one or more lateral pads is coupled to the center pad via one of the one or more hammock attachments, and where each of the one or more hammock attachments may include a length of flexible material to adjust the extension of the respective lateral pad relative to the center pad. System where each of the one or more lateral flaps is configured to receive one of the lateral pads. System where the trunk pad may include an opening and an insert, and where the trunk pad is configured to receive the insert, the insert configured to support at least a portion of the patient. System where each of the one or more lateral flaps is translatable such that the position of each of the one or more lateral flaps relative to the platform is adjustable in at least one of the lateral direction and the medial direction. System where each of the one or more lateral flaps is rotatable such that the pitch of the one or more lateral flaps relative to the platform is adjustable. System where each of the one or more lateral flaps is translatable and rotatable via a single control. System where each of the one or more lateral flaps is rotatable such that the pitch of the one or more lateral flaps relative to the platform is adjustable. System where each of the one or more lateral flaps is rotatable such that the position of each of the one or more lateral flaps relative to the platform is adjustable in at least one of an anterior direction and a posterior direction. Implementations of the described techniques may include hardware, a method or process, or a computer tangible medium.

Features described above in respect of the preceding aspect may also apply to the next aspect.

In one general aspect, lift may include a slider. Lift may also include one or more linkages configured to couple the slider to a spar configured to support at least one leg of a patient. Lift may furthermore include a first spring configured to compress and expand. Lift may in addition include a ball nut configured to compress the spring. Lift may moreover include where the slider is configured to translate as the spring is expanded or compressed to provide lift assist for the spar. Other embodiments of this aspect include corresponding computer systems, apparatus, and computer programs recorded on one or more computer storage devices, each configured to perform the actions of the methods.

Implementations may include one or more of the following features.

Mechanism may include: a screw, where the ball nut is configured to translate along the screw. Mechanism where the mechanism further may include: a gearbox and one or more second springs, each second spring having a mainspring coupled to the screw by the gearbox, the one or more second springs configured to provide lift assist for the spar to articulate the spar in an upward pitch direction. Mechanism where the one or more second springs is compressed to provide lift assist for the spar to articulate the spar in the upward pitch direction. Mechanism where the one or more second springs may include a plurality of springs stacked on top of one another to provide torque for the lift assist mechanism. Mechanism where the slider is coupled to the ball nut such that the slider is configured to translate as the ball nut translates along the screw. Mechanism where the slider is configured to translate along a pivot post to provide lift assist for the spar. Mechanism where the first spring is a coil spring. Mechanism where the one or more linkages is coupled to a spar mount configured to receive the spar, the one or more linkages configured to articulate the spar mount in a pitch direction to provide lift assist for the spar. Mechanism where the first spring is configured to be compressed to allow the spar to articulate the downward pitch direction. Implementations of the described techniques may include hardware, a method or process, or a computer tangible medium.

Features described above in respect of any of the preceding aspects may also apply to the next aspect.

In one general aspect, attachment system may include an universal attachment interface (UAI) port configured to receive two or more table modules, each of the table modules being distinct from another one of the table modules. Attachment system may also include one or more UAI plugs, each of the UAI ports configured to receive the one or more UAI plugs, each of the table modules having at least one of the one or more UAI plugs. Other embodiments of this aspect include corresponding computer systems, apparatus, and computer programs recorded on one or more computer storage devices, each configured to perform the actions of the methods.

Implementations may include one or more of the following features.

System where each of the one or more UAI ports may include an electrical connection. System where each of the table modules may include an UAI lock configured to retain the UAI plug in the UAI port. System where each of the UAI ports may include a dog-bone shape. System where each of the UAI ports may include first opening, a second opening, and a third opening between the first opening and the second opening, where the first opening and the second opening are substantially spherical, and where the third opening is substantially rectangular. System where the first opening and the second opening are configured to guide the UAI plug of the respective table module such that the UAI port receives the table module. System where the first opening and the second opening each may include a bevel shape configured to guide the UAI plug of the respective table module such that the UAI port receives the table module. Implementations of the described techniques may include hardware, a method or process, or a computer tangible medium.

Features described above in respect of any of the preceding aspects may also apply to the next aspect.

In one general aspect, system may include a surgery table having a distal end and a proximal end, the surgery table having a platform configured to support at least a portion of the patient. System may also include a spar base positioned beneath the platform at the distal end and configured to translate along the surgery table between the distal end and the proximal end of the surgery table. System may furthermore include a spar extending from the spar base and configured to support at least one leg of the patient, the spar having a proximal end, a distal end, and a center joint, the distal end and the proximal end of the spar coupled together by the center joint. System may in addition include where the center joint is configured to actuate while the spar base translates along the surgery table between the distal end and the proximal end of the surgery table. Other embodiments of this aspect include corresponding computer systems, apparatus, and computer programs recorded on one or more computer storage devices, each configured to perform the actions of the methods.

Implementations may include one or more of the following features.

System where the distal end of the spar is distal to the proximal end of the spar, the proximal end of the spar coupled to the spar base. System where the center joint is configured to raise or lower the distal end of the spar such that the distal end is at an angle of about 70 degrees relative to the proximal end of the spar. System may include a manual lock proximate the center joint, where the center joint is configured to be locked via the manual lock. System where the center joint is configured to be released via one or more joint controls remote from the center joint. System where the distal end of the spar may include a handle configured to articulate the spar, the handle having the one or more joint controls. System where the manual lock is configured to be in a sterile space. System where the proximal end of the spar may include a gear configured to control the orientation of the proximal end. System where the proximal end of the spar may include one or more links, and where the center joint is coupled to a passive link that is coupled to the gear via the one or more links. System where the one or more links may include a first link and a second link, and where the gear is configured to control the orientation of the first link. System may include a rotating link, where the second link is coupled to the rotating link, and where the rotating link, the passive link, the first link, and the second link form a 4-bar linkage configured to rotate around the passive link. System where the spar base is configured to translate along the surgery table a distance between the distal end and the proximal end of the surgery table. System where the spar base is configured to articulate the spar in a pitch direction. System where the distal end of the spar may include a foot support module configured to support at least one foot of the at least one leg of the patient. System where the foot support module extends outwardly from the spar in a lateral direction such that the spar is medially offset from at least a portion of the leg of the patient. System where the foot support module is in a working space. System may include: a foot support configured to support the at least one foot of the at least one leg of the patient, where the foot support module may include a gripping mechanism configured to grip the foot support. System where the foot support is in a sterile space. System where the foot support module is in a working space. System where the foot support module and the foot support are coupled via a mechanical coupling that is configured to allow the foot support to pitch about a rotational axis relative to the foot support module. System where the foot support further may include one or more magnets to actuate the mechanical coupling, the one or more magnets being disposed in a sterile space. System where the one or more magnets is configured to generate a magnetic field that extends to the foot support module in a working space. System where the foot support may include a user interface paddle configured to release the foot support from the foot support module. System where the foot support may include an user interface paddle configured to release the foot support from the foot support module. System where the foot support may include an user interface paddle configured to release the foot support from the foot support module, and where the user interface paddle may include a detent configured to lock the user interface paddle in one or more positions. System where the foot support may include one or more springs, where the user interface paddle may include a first position and a third position, the foot support being biased to the first position and the third position by the one or more springs, and where the detent is configured to secure the user interface paddle in at least one of the first position or the third position. System where the third position is outward of the first position. System where the detent may include a flat portion configured to receive the one or more springs such that pressure from the one or more springs secures the user interface paddle. System where, in the first position, the one or more magnets does not generate the magnetic field and, in a second position, the one or more magnets generates the magnetic field, where the foot support may include a rod having mu metal, where the rod is configured to alternately magnetize in the presence of a magnetic field and essentially demagnetize in the absence of a magnetic field, and where the magnetic field is configured to actuate the foot support such that the rod is gripped and in the absence of a magnetic field is configured to release the foot support such that the rod is released. System where when the user interface paddle is in the second position, the distal end of the spar is configured to fold and the proximal end of the spar is configured to translate substantially simultaneously. Foot support where the user interface paddle may include a detent configured to lock the user interface paddle in one or more positions. Foot support where the foot support further may include one or more springs, where the user interface paddle may include a third position in which the one or more magnets does not generate the magnetic field, the foot support being biased to the first position and the third position by the one or more springs, and where the detent is configured to secure the user interface paddle in at least one of the first position or the third position. Foot support where the third position is outward of the first position. Foot support where the detent may include a flat portion configured to receive the one or more springs such that pressure from the one or more springs secures the user interface paddle. Foot support where when the user interface paddle is in the second position, a distal end of a spar configured to support the at least one leg of a patient for the surgical procedure is configured to fold and a proximal end of the spar is configured to translate substantially simultaneously, and where the foot support is coupled to the spar. System where the foot support may include a rod having the rotational axis and configured to be received by the gripping mechanism. System where the rod may include mu metal configured to alternately magnetize in the presence of a magnetic field, and essentially demagnetize in the absence of a magnetic field, and where the gripping mechanism is configured to be actuated by the magnetic field such that the rod is gripped, and the gripping mechanism is configured to release the rod in the absence of a magnetic field, the foot support being rotatable in a pitch direction when the gripping mechanism is actuated and released. System where the gripping mechanism is configured to rotate inward when actuated to grip the foot support, and where the gripping mechanism is configured to rotate outward when released to release the foot support. System where the foot support may include one or more magnets configured to generate the magnetic field. System where the foot support may include one or more springs and a user interface paddle having a first position in which the one or more magnets does not generate the magnetic field and a second position in which the one or more magnets generates the magnetic field, the foot support being biased to the first position by the one or more springs. System where, in the second position, the one or more magnets are adjacent to a metal sidewall having a mu metal conduit having mu metal, the mu metal conduit coupled to the rod having mu metal such that the magnetic field generated by the one or more magnets is extended to the rod via the mu metal conduit. System where the foot support module may include a magnetic sensor configured to actuate the gripping mechanism when the magnetic field is present, where the magnetic field is generated in a sterile space, and where the foot support module is in a working space, the working space and the sterile space being separated by a drape. System where when the user interface paddle is in the second position, the system is configured to substantially simultaneously fold the distal end of the spar and translate the proximal end of the spar via the spar base. System where the distal end of the spar is configured to fold substantially simultaneously with the translation of the spar base when the center joint is actuated. System where the spar is configured to fold up when the center joint is actuated. System where the center joint is configured to lower such that the distal end of the spar is at an angle of about 70 degrees relative to the proximal end of the spar. System where the spar is configured to raise relative to the spar being in the straight spar configuration as the spar folds up. System where the surgery table further may include a pelvic port at the distal end of the surgery table, the pelvic port is configured to receive a plurality of attachments configured to support a pelvis of the patient. System may include a pelvic port adapter, where the pelvic port may include a rail, and where the pelvic port adapter may include a track, the track configured to receive the rail such that the pelvic port adapter is coupled to the pelvic port. System where the pelvic port adapter is configured to receive the plurality of attachments. System where the pelvic port adapter may include a protrusion. System where each of the plurality of attachments may include a receptacle configured to receive the protrusion of the pelvic port adapter. System where the plurality of attachments may include a knee ledge board configured to support at least a portion of the patient. System where the knee ledge board may include a receptacle configured to receive the protrusion of the pelvic port adapter. System where the knee ledge board may include one or more grooves configured to support at least a portion of the patient. System where the knee ledge board is substantially radiolucent. System where the knee ledge board may include essentially of carbon fiber. System may include: a knee ledge board pad, where the knee ledge board is configured to receive the knee ledge board pad, the knee ledge board pad having a foam configured to support at least a portion of the patient. System where the knee ledge board pad is disposable. System may include one or more spar boards, where the one or more spar boards is configured to support at least a portion of the leg of the patient. System where the one or more spar boards is coupled to at least one of the distal end and the proximal end of the spar. System where the one or more spar boards is configured to couple to the spar from at least one of a lateral direction and a medial direction. System where the one or more spar boards is radiolucent. System where the one or more spar boards may include essentially of carbon fiber. System where each of the one or more spar boards may include an extension configured to extend and retract relative to the respective spar board to adjust the length of the respective spar board. System where each of the one or more spar boards may include a clamp configured to couple the respective spar board to the spar. System where the clamp is radiolucent. System where the clamp may include essentially of carbon fiber. System where the clamp may include a profile having a substantially hexagonal shape. System may include: one or more spar board pads, each of the spar board pads configured to be disposed on at least a portion of one of the one or more spar boards, each of the one or more spar board pads configured to support at least a portion of the patient. System where each of the one or more spar board pads may include a pad rollout, the pad rollout configured to extend and retract relative to the respective spar board pad to adjust the length of the respective spar board pad. System may include: one or more pivot boards configured to support at least a portion of the leg of the patient. System where the one or more pivot boards is coupled to the proximal end of the spar. System where the one or more pivot boards is configured to rotate relative to an axis extending along the spar when the spar is in a straight configuration. System where the distal end of the spar is configured to fold substantially simultaneously with the translation of the spar base when the center joint is actuated, and where the one or more pivot boards is rotated as the distal end is folded and the proximal end is translated. System where the one or more pivot boards is rotated such that the one or more pivot boards is on a lateral side of the spar. System where the one or more pivot boards is radiolucent. System where the one or more pivot boards may include essentially of carbon fiber. System may include: one or more drapes for the system to separate a sterile space and a working space. System where the distal end of the spar may include a foot support module configured to support at least one foot of the at least one leg of the patient. System may include: a foot support configured to support the at least one foot of the at least one leg of the patient, where the foot support module may include a gripping mechanism configured to grip the foot support. System where the foot support module is in the working space, and where the foot support is in the sterile space. System where the one or more drapes may include one or more lower drapes configured to contact the working space, and one or more top drapes configured to contact the one or more lower drapes and the sterile space. System where the one or more drapes may include a first lower drape having a leg opening configured to wrap around a first leg of the patient, a fabric portion, and a film portion. System where the distal end of the spar further may include a handle configured to allow a user to articulate the spar, the film portion being disposed over the handle such that the user may view the handle through the one or more drapes. System where the one or more drapes may include a second lower drape having a leg opening configured to wrap around a second leg of the patient, a first leg portion configured to cover at least a portion of the first leg of the patient, and a second leg portion configured to be disposed between the first leg and the second leg of the patient. System where the one or more drapes may include a top drape configured to contact the sterile space, the top drape having one or more arm portions configured to cover one or more arms of the patient as the one or more arms extend laterally from the patient. System where the one or more drapes may include a top drape configured to contact the sterile space, the top drape having a film portion configured to be disposed over a portion of the surgery table having one or more controls such that a user may view the controls through the one or more drapes. System where the proximal end may include a locking mechanism configured to lock the orientation of the proximal end. System where one or more links may include a first link and a second link, and where the locking mechanism is configured to lock the first link. System where the locking mechanism may include a pair of gears configured to alternately rotate and lock the first link. System where the proximal gear and the distal gear are coupled to one another such that the distal rotational shaft is configured to rotate relative to the proximal rotational shaft and locking the proximal rotational shaft locks the distal rotational shaft to lock the first link. System where the slider-crank mechanism may include an electromagnetic brake, a ball screw, a ball nut coupled to the ball screw, and a slider link coupled to the ball nut and the proximal rotational shaft, where the ball screw is configured to rotate such that the ball nut translates vertically when the electromagnetic brake is unlocked, and where the ball screw is configured to stop rotating when the electromagnetic brake is locked such that the slider link is locked in position to lock the proximal rotational shaft to lock the first link. System where the slider-crank mechanism may include an electromagnetic brake and a slider link coupled to the proximal rotational shaft, where the slider link is configured to rotate the proximal rotational shaft when the electromagnetic brake is unlocked, and where the slider link is locked in position when the electromagnetic brake is locked such that the proximal rotational shaft is locked to lock the first link. System where the locking mechanism further may include a slider-crank mechanism configured to control the orientation of the proximal end. System where the locking mechanism may include a proximal rotational shaft, a distal rotational shaft coupled to the first link, and a pair of gears having a proximal gear coupled to the proximal rotational shaft and a distal gear coupled to the distal rotational shaft. Implementations of the described techniques may include hardware, a method or process, or a computer tangible medium.

Features described above in respect of any of the preceding aspects may also apply to the next aspect.

In one general aspect, spar may include a proximal end coupled to a spar base of a surgery table. Spar may also include a center joint. Spar may furthermore include a distal end coupled to the proximal end of the spar by the center joint. Spar may in addition include where the proximal end is configured to translate along the surgery table between the distal end and the proximal end of the surgery table as the center joint is actuated. Other embodiments of this aspect include corresponding computer systems, apparatus, and computer programs recorded on one or more computer storage devices, each configured to perform the actions of the methods.

Implementations may include one or more of the following features.

Spar where the distal end of the spar may include a foot support module configured to support the at least one foot of the at least one leg of the patient. Spar where the foot support module extends outwardly from the spar in a lateral direction such that the spar is medially offset from at least a portion of the leg of the patient. Spar where the foot support module is in a working space. Spar where the distal end of the spar may include a foot support, and where the foot support module may include a gripping mechanism configured to grip the foot support. Spar where the foot support is in a sterile space. Spar where the foot support module is in a working space. Spar where the foot support module and the foot support are coupled via a mechanical coupling that is configured to allow the foot support to pitch about a rotational axis relative to the foot support module. Spar may include one or more magnets to actuate the mechanical coupling, the one or more magnets are disposed in the sterile space. Spar where the one or more magnets is configured to generate a magnetic field that extends to the working space. Spar where the foot support may include a user interface paddle configured to release the foot support from the foot support module. Spar where the foot support may include a rod having the transverse axis and configured to be received by the gripping mechanism. Spar where the rod may include mu metal configured to alternately magnetize in the presence of a magnetic field and essentially demagnetize in the absence of a magnetic field, and where the gripping mechanism is configured to be actuated by the magnetic field such that the rod is gripped, and the gripping mechanism is configured to release the rod in the absence of a magnetic field, the foot support being rotatable in a pitch direction when the gripping mechanism is actuated and released. Spar where the gripping mechanism is configured to rotate inward when actuated to grip the foot support, and where the foot support is configured to rotate outward when released to release the foot support. Spar where the foot support may include one or more magnets configured to generate the magnetic field. Spar where the foot support may include one or more springs and a user interface paddle having a first position in which the one or more magnets does not generate the magnetic field and a second position in which the one or more magnets generates the magnetic field, the foot support being biased to the first position by the one or more springs. Spar where, in the second position, the one or more magnets contact a metal sidewall having a mu metal conduit having mu metal, the mu metal conduit coupled to the rod having mu metal such that the magnetic field generated by the one or more magnets is extended to the rod via the mu metal conduit. Spar where the foot support module may include a magnetic sensor configured to actuate the gripping mechanism when the magnetic field is present. Spar where when the user interface paddle is in the second position, the distal end of the spar is configured to fold and the proximal end of the spar is configured to translate substantially simultaneously. Spar where the foot support may include an user interface paddle configured to release the foot support from the foot support module, and where the user interface paddle may include a detent configured to lock the user interface paddle in one or more positions. Spar where the foot support may include one or more springs, where the user interface paddle may include a first position and a third position, the foot support being biased to the first position and the third position by the one or more springs, and where the detent is configured to secure the user interface paddle in at least one of the first position or the third position. Spar where the third position is outward of the first position. Spar where the detent may include a flat portion configured to receive the one or more springs such that pressure from the one or more springs secures the user interface paddle. Spar where, in the first position, the one or more magnets does not generate the magnetic field and, in a second position, the one or more magnets generates the magnetic field, where the foot support may include a rod having mu metal, where the rod is configured to alternately magnetize in the presence of a magnetic field and essentially demagnetize in the absence of a magnetic field, and where the magnetic field is configured to actuate the foot support such that the rod is gripped and in the absence of a magnetic field is configured to release the foot support such that the rod is released. Spar where the user interface paddle, in the second position, the distal end of the spar is configured to fold and the proximal end of the spar is configured to translate substantially simultaneously. Spar where the distal end of the spar is configured to fold substantially simultaneously with the translation of the spar base when the center joint is actuated. Spar where the spar is configured to fold up when the center joint is actuated. Spar where the center joint is configured to lower such that the distal end of the spar is at an angle of about 70 degrees relative to the proximal end of the spar. Spar where the spar is configured to raise relative to the spar being in the straight spar configuration as the spar folds up. Spar may include one or more spar boards, where the one or more spar boards is configured to support at least a portion of the leg of the patient. Spar where the one or more spar boards is coupled to at least one of the distal end and the proximal end of the spar. Spar where the one or more spar boards is configured to couple to the spar from at least one of a lateral direction and a medial direction. Spar where the one or more spar boards is radiolucent. Spar where the one or more spar boards may include essentially of carbon fiber. Spar where each of the one or more spar boards may include an extension configured to extend and retract relative to the respective spar board to adjust the length of the respective spar board. Spar where each of the one or more spar boards may include a clamp configured to couple the respective spar board to the spar. Spar where the clamp is radiolucent. Spar where the clamp may include essentially of carbon fiber. Spar where the clamp may include a profile having a substantially hexagonal shape. Spar may include: one or more spar board pads, each of the spar board pads configured to be disposed on at least a portion of one of the one or more spar boards, each of the one or more spar board pads configured to support at least a portion of the patient. Spar where each of the one or more spar board pads may include a pad rollout, the pad rollout configured to extend and retract relative to the respective spar board pad to adjust the length of the respective spar board pad. Spar may include: one or more pivot boards configured to support at least a portion of the leg of the patient. Spar where the one or more pivot boards is coupled to the proximal end of the spar. Spar where the one or more pivot boards is configured to rotate relative to an axis extending along the spar when the spar is in a straight configuration an axis extending along the spar when the spar is in a straight configuration. Spar where the distal end of the spar is configured to fold substantially simultaneously with the translation of the spar base when the center joint is actuated, and where the one or more pivot boards is rotated as the distal end is folded and the proximal end is translated. Spar where the one or more pivot boards is rotated such that the one or more pivot boards is on a lateral side of the spar. Spar where the one or more pivot boards is radiolucent. Spar where the one or more pivot boards may include essentially of carbon fiber. Implementations of the described techniques may include hardware, a method or process, or a computer tangible medium.

Features described above in respect of any of the preceding aspects may also apply to the next aspect.

In one general aspect, foot support mechanism may include a foot support configured to support at least one foot of at least one leg of a patient. Foot support mechanism may also include a foot support module having a gripping mechanism configured to grip the foot support. Mechanism may furthermore include a mechanical coupling configured to couple the foot support to the foot support module, the mechanical coupling configured to magnetically actuate the gripping mechanism and to allow the foot support to pitch about a transverse axis relative to the foot support module. Other embodiments of this aspect include corresponding computer systems, apparatus, and computer programs recorded on one or more computer storage devices, each configured to perform the actions of the methods.

Implementations may include one or more of the following features. Foot support mechanism where the foot support module is in a working space. Foot support mechanism where the foot support is in a sterile space. Foot support mechanism may include one or more magnets to actuate the mechanical coupling, the one or more magnets are disposed in the sterile space. Foot support mechanism where the one or more magnets is configured to generate a magnetic field that extends to a working space. Foot support mechanism where a spar configured to support the at least one leg of the patient may include the foot support module. Foot support mechanism where the spar may include a proximal end, a center joint, and a distal end coupled to the proximal end of the spar by the center joint, where, as the gripping mechanism is actuated, the distal end of the spar is configured to fold substantially simultaneously as the proximal end of the spar translates relative to the surgery table when the center joint is actuated. Foot support mechanism where the foot support may include an user interface paddle configured to release the foot support from the foot support module. Foot support mechanism where the foot support may include a rod having the transverse axis and configured to be received by the gripping mechanism. Foot support mechanism where the rod may include mu metal configured to alternately magnetize in the presence of a magnetic field and essentially demagnetize in the absence of a magnetic field, and where the magnetic field is configured to actuate the gripping mechanism such that the rod is gripped and in the absence of a magnetic field is configured to release the gripping mechanism such that the rod is released. Foot support mechanism where the gripping mechanism is configured to rotate inward when actuated to grip the foot support, and where the foot support is configured to rotate outward when released to release the foot support. Foot support mechanism where the foot support may include one or more magnets configured to generate the magnetic field. Foot support mechanism where the foot support may include one or more springs and an user interface paddle having a first position in which the one or more magnets does not generate the magnetic field and a second position in which the one or more magnets generates the magnetic field, the foot support being biased to the first position by the one or more springs. Foot support mechanism where the foot support may include an user interface paddle configured to release the foot support from the foot support module, and where the user interface paddle may include a detent configured to lock the user interface paddle in one or more positions. Foot support mechanism where the foot support may include one or more springs, where the user interface paddle may include a first position and a third position, the foot support being biased to the first position and the third position by the one or more springs, and where the detent is configured to secure the user interface paddle in at least one of the first position or the third position. Foot support mechanism where the third position is outward of the first position. Foot support mechanism where the detent may include a flat portion configured to receive the one or more springs such that pressure from the one or more springs secures the user interface paddle. Foot support mechanism where the foot support may include one or more magnets configured to generate a magnetic field such that the mechanical coupling is configured to magnetically actuate the gripping mechanism, where, in the first position, the one or more magnets does not generate the magnetic field and, in a second position, the one or more magnets generates the magnetic field, where the foot support may include a rod having mu metal, where the rod is configured to alternately magnetize in the presence of a magnetic field and essentially demagnetize in the absence of a magnetic field, and where the magnetic field is configured to actuate the foot support such that the rod is gripped and in the absence of a magnetic field is configured to release the foot support such that the rod is released. Foot support mechanism where when the user interface paddle is in the second position, the distal end of the spar is configured to fold and the proximal end of the spar is configured to translate substantially simultaneously. Implementations of the described techniques may include hardware, a method or process, or a computer tangible medium.

In one general aspect, foot support mechanism may include The foot support mechanism. Other embodiments of this aspect include corresponding computer systems, apparatus, and computer programs recorded on one or more computer storage devices, each configured to perform the actions of the methods.

Implementations may include one or more of the following features. Foot support mechanism where the foot support module may include a magnetic sensor configured to actuate the gripping mechanism when the magnetic field is present. Foot support mechanism where the user interface paddle, in the second position, is configured to fold the distal end of the spar and translate the proximal end of the substantially simultaneously.

Features described above in respect of any of the preceding aspects may also apply to the next aspect.

In one general aspect, surgery table may include a platform configured to support at least a portion of a patient for a surgical procedure. Surgery table may also include a spar base positioned beneath the platform. Table may furthermore include a spar for supporting at least one leg of the patient, the spar extending from the spar base, the spar having a proximal end, a distal end, and a center joint, the distal end and the proximal end of the spar coupled together by the center joint. Table may in addition include where the center joint is configured to actuate to allow direct inferior access to the knee of a patient when the knee is bent down and the spar is bent up. Other embodiments of this aspect include corresponding computer systems, apparatus, and computer programs recorded on one or more computer storage devices, each configured to perform the actions of the methods.

Implementations may include one or more of the following features.

Surgery table where the surgery table is configured to allow lateral access to the knee of a patient. Surgery table where the surgery table is configured to allow medial access to the knee of a patient. Surgery table where the spar may include a proximal end coupled to the spar base, a distal end, and a center joint, the distal end and the proximal end of the spar coupled together by the center joint. Surgery table where the distal end of the spar does not may include a traction mechanism. Surgery table where the distal end of the spar may include a foot support module configured to support at least one foot of the at least one leg of the patient, and where the distal end of the spar is configured to reach a position proximate a ground surface supporting the system when the spar is articulated in a downward pitch direction such that the foot of the patient is proximate the ground surface. Surgery table where the distal end of the spar may include a handle configured to allow a user to articulate the spar. Implementations of the described techniques may include hardware, a method or process, or a computer tangible medium.

Features described above in respect of any of the preceding aspects may also apply to the next aspect.

In one general aspect, spar may include a proximal end coupled to a spar base of a surgery table.

Spar may also include a center joint. Spar may furthermore include a distal end coupled to the proximal end of the spar by the center joint. Spar may in addition include where the center joint is configured to actuate to allow direct inferior access to the knee of a patient when the knee is bent down and the spar is bent up. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

Implementations may include one or more of the following features.

Surgery table where the distal end of the spar does not may include a traction mechanism. Surgery table where the distal end of the spar may include a foot support module configured to support at least one foot of the at least one leg of the patient, and where the distal end of the spar is configured to reach a position proximate a ground surface supporting the system when the spar is articulated in a downward pitch direction such that the foot of the patient is proximate the ground surface. Surgery table where the distal end of the spar may include a handle configured to allow a user to articulate the spar. Implementations of the described techniques may include hardware, a method or process, or a computer tangible medium.

Features described above in respect of any of the preceding aspects may also apply to the next aspect.

In one general aspect, surgical method may include supporting at least a portion of a leg of a patient on a spar extending from a surgery table, the spar having a proximal end, and a distal end, and a center joint, the proximal end of the spar coupled to the surgery table, and the distal end and the proximal end of the spar coupled together by the center joint. Surgical method may also include flexing the spar by releasing the center joint. Method may furthermore include moving the spar relative to the surgery table to flex the leg of the patient. Method may in addition include allowing inferior access to a knee of the leg. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

In one general aspect, surgical method may include supporting at least a portion of a leg of a patient on a spar extending from a surgery table, the spar having a proximal end, and a distal end, and a center joint, the proximal end of the spar coupled to the surgery table, and the distal end and the proximal end of the spar coupled together by the center joint. Surgical method may also include generating a magnetic field in a sterile space on a first side of a drape, the magnetic field generated by one or more magnets of a foot support at the distal end of the spar, the foot support configured to support the at least one foot of the at least one leg of the patient. Method may furthermore include extending the magnetic field to a working space on a second side of the drape opposing the first side, the magnetic field extended via mu metal may include by the foot support. Method may in addition include actuating a magnetic sensor in the working space. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

Implementations may include one or more of the following features.

Surgical method may include: flexing the spar by releasing the center joint; and moving the spar relative to the surgery table to flex the leg of the patient. Surgical method may include: allowing inferior access to a knee of the leg. Surgical method where the distal end of the spar may include a foot support module configured to support the at least one foot of the at least one leg of the patient. Surgical method where the foot support module may include the magnetic sensor. Surgical method where the foot support module is in the working space. Surgical method where the foot support may include a rod having the mu metal. Surgical method where the rod is configured to alternately magnetize in the presence of a magnetic field and essentially demagnetize in the absence of a magnetic field, and where the magnetic field is configured to actuate the foot support such that the rod is gripped and in the absence of a magnetic field is configured to release the foot support such that the rod is released. Implementations of the described techniques may include hardware, a method or process, or a computer tangible medium.

Features described above in respect of any of the preceding aspects may also apply to the next aspect.

In one general aspect, foot support may include one or more magnets configured to generate a magnetic field that extends across a drape for the surgical procedure. Foot support may also include a user interface paddle having a first position in which the one or more magnets does not generate the magnetic field and a second position in which the one or more magnets generates the magnetic field. Support may furthermore include a rod having mu metal configured to alternately magnetize in the presence of a magnetic field, and essentially demagnetize in the absence of a magnetic field. Other embodiments of this aspect include corresponding computer systems, apparatus, and computer programs recorded on one or more computer storage devices, each configured to perform the actions of the methods.

Features described above in respect of any of the preceding aspects may also apply to the next aspect.

Implementations may include one or more of the following features.

Foot support where the rod may include a rotational axis about which the foot support is configured to rotate in a pitch direction. Foot support may include: one or more springs configured to bias the user interface paddle to the first position. Foot support may include: a sidewall having a mu metal conduit having mu metal, the mu metal conduit coupled to the rod having mu metal such that the magnetic field generated by the one or more magnets is extended to the rod via the mu metal conduit. Implementations of the described techniques may include hardware, a method or process, or a computer tangible medium.

Features described above in respect of any of the preceding aspects may also apply to the next aspect.

In one general aspect, spar may include a proximal end coupled to a spar base of a surgery table. Spar may also include a distal end coupled to the proximal end of the spar. Spar may furthermore include where the spar may include a straight spar configuration in which spar is not pitched or yawed. Spar may in addition include where the spar is configured to pitch in an upward direction from the straight spar configuration between about 10 degrees and about 30 degrees, pitch in a downward direction from the straight spar configuration between about 20 degrees and about 40 degrees, and yaw from the straight spar configuration between about 10 degrees and about 30 degrees. Other embodiments of this aspect include corresponding computer systems, apparatus, and computer programs recorded on one or more computer storage devices, each configured to perform the actions of the methods.

Implementations may include one or more of the following features. Spar where the spar is configured to pitch in the upward direction from the straight spar configuration about 20 degrees. Spar where the spar is configured to pitch in the downward direction from the straight spar configuration about 36 degrees. Spar where the spar is configured to yaw from the straight spar configuration inward about 20 degrees. Spar where the spar is configured to yaw from the straight spar configuration outward about 40 degrees. Spar where the spar extends along a horizontal plane of the surgical table and along axes generally parallel to a centerline of the surgical table in the straight spar configuration, and where yaw inward and outward of the spar is relative to the centerline. Implementations of the described techniques may include hardware, a method or process, or a computer tangible medium.

Features described above in respect of any of the preceding aspects may also apply to the next aspect.

In one general aspect, a surgical method may include supporting at least a portion of a leg of a patient on a foot support; placing a drape over a foot support module coupled to a distal end of a spar extending from a surgery table, the spar comprising a proximal end and an opposing distal end, the proximal end of the spar coupled to the surgery table, the drape defining a sterile space above the drape and a working space below the drape; coupling the foot support to the foot support module with the drape in between the foot support and the foot support module such that the foot support is in the sterile space and the foot support module is in the working space; generating a magnetic field in the sterile space with one or more magnets of the foot support, generating comprising moving the one or more magnets proximal to a mu-metal pathway comprised within the foot support and extending the magnetic field through the mu-metal pathway to a magnetic field terminus; placing the magnetic field terminus in proximity to a magnetic sensor comprised within the foot support module and extending the magnetic field across the drape from the sterile space to the working space; and actuating the magnetic sensor in the working space via the magnetic field extending from the magnetic field terminus to the magnetic sensor

Implementations may include one or more of the following features. The foot support may include a foot support attachment comprising the magnetic field terminus, and the magnetic field may actuate the foot support module to grip the foot support attachment. The method may include moving a user interface of the foot support attachment from a first position in which the one or more magnets are not proximal to the mu-metal pathway and do not generate the magnetic field at the magnetic field terminus, to a second position in which the one or more magnets are proximal to the mu-metal pathway and extend the magnetic field to the magnetic field terminus, the user interface optionally biased to the first position and optionally including a detent configured to lock the user interface in at least one of the first position or a third position outward of the second position. In the second position of the user interface, a distal end of the spar may be configured to fold and a proximal end of the spar may be configured to translate substantially simultaneously. The magnetic field terminus may include a rotational axis about which the foot support is configured to rotate in a pitch direction. The method may include releasing a joint disposed between and configured to couple the proximal end to the distal end of the spar and flexing the spar generally in a posterior direction by releasing the joint such that an inferior window is formed proximate to the spar to allow access to a knee of the leg generally from an inferior direction. The magnets may actuate a mechanical coupling configured to couple the foot support to the foot support module. The foot support module may include a switch configured to deenergize the magnetic sensor.

In one general aspect, a system may include a surgery table having a distal end and a proximal end opposing the distal end, and a platform configured to support at least a portion of a patient; a spar base coupled to the surgery table and positioned beneath the platform; and a spar having a proximal end coupled to and extending from the spar base and a distal end. The system may further include a foot support coupled to and disposed at the distal end of the spar, the foot support comprising a mu-metal pathway and a magnetic field terminus comprising mu-metal and one or more magnets opposite the magnetic field terminus, a foot support module coupled to the distal end of the spar and comprising a magnetic sensor, and a drape configured to be placed over the foot support module such that the drape defines a sterile space above the drape and a working space below the drape and the foot support is configured to be coupled to the foot support module with the drape in between the foot support and the foot support module, the foot support in the sterile space and the foot support module in the working space. The magnets may be configured to generate a magnetic field in the sterile space, to selectively move proximal to the mu-metal pathway and extend the magnetic field through the mu-metal pathway to the magnetic field terminus, the magnetic field terminus configured to be moved proximate to the magnetic sensor and extend the magnetic field across the drape from the sterile space to the working space, and the magnetic sensor configured to be actuated in the working space via the magnetic field extending from the magnetic field terminus to the magnetic sensor.

Implementations may include one or more of the following features. The foot support may include a foot support attachment comprising the magnetic field terminus such that the magnetic field actuates the foot support module to grip the foot support attachment. The foot support may include a user interface configured to be moved from a first position in which the magnets are not proximal to the mu-metal pathway and do not generate the magnetic field at the magnetic field terminus, to a second position in which the magnets are proximal to the mu-metal pathway and extend the magnetic field to the magnetic field terminus, the user interface optionally biased to the first position and optionally including a detent configured to lock the user interface in at least one of the first position or a third position outward of the second position. In the second position of the user interface, a distal end of the spar may be configured to fold and a proximal end of the spar may be configured to translate substantially simultaneously. The magnetic field terminus may include a rotational axis about which the foot support is configured to rotate in a pitch direction. The system may further include a joint disposed between and configured to couple the proximal end to the distal end of the spar, the joint configured to release to flex the spar generally in a posterior direction such that an inferior window is formed proximate to the spar to allow access to a knee of the leg generally from an inferior direction.

Features described above in respect of any of the preceding aspects may also apply to the next aspect.

In one general aspect, a spar may include a proximal end configured to couple to a surgery table, a distal end opposing the proximal end, a joint disposed between and configured to couple the proximal end to the distal end, the joint further configured to rotate and flex the spar in a generally posterior direction when coupled to the surgery table, the joint comprising an angle between the proximal end and the distal end configured to be reduced as the joint is rotated, and a foot support coupled to and disposed at the distal end of the spar and configured to secure a foot of a leg of a patient, where the spar is configured to flex in the generally posterior direction as the leg is flexed in a generally anterior direction opposing the generally posterior direction such that the leg is able to flex away from the spar and without the spar being directly between an upper leg and a lower leg of the leg when the leg is flexed.

Implementations may include one or more of the following features. Spar where the joint is configured to rotate to flex the spar to reduce the angle between the proximal end and the distal end to an acute angle such that an inferior window is formed proximate to the spar to allow access to a knee of the leg generally from an inferior direction. Spar where the joint is configured to rotate in a plane generally parallel to a plane containing the leg such that the spar and the leg flex in generally parallel planes. Spar where the joint is configured to be locked to fix the distal end of the spar relative to the proximal end of the spar at a plurality of angles, the plurality of angles comprising the angle. Spar further including a control disposed at the distal end of the spar, where the joint is configured to be unlocked by the control. Spar further including a handle disposed at the distal end of the spar, the handle comprising the control. Spar where the spar comprises a straight spar configuration in which the spar is not pitched or yawed, where the straight spar configuration comprises the spar extending generally along a horizontal plane of the surgical table and along an axis generally parallel to a centerline of the surgical table, and where the spar is configured to yaw from the straight spar configuration inward and outward relative to the centerline. Spar where the spar is configured to yaw from the straight spar configuration inward about 20 degrees, and where the spar is configured to yaw from the straight spar configuration outward about 40 degrees. Spar where the spar is configured to yaw outward such that a medial window and a lateral window are formed proximate to the spar to allow access to the knee of the leg from generally a medial direction and generally a lateral direction. Spar where the joint is configured to rotate to flex the spar to reduce the angle between the proximal end and the distal end to an acute angle to allow further access to the knee of the leg from a generally inferior direction. Spar where, as the spar is flexed, the spar is configured to translate in a superior direction relative to the surgery table and lower in generally a posterior direction such that a height of a knee of the leg of the patient is generally maintained as the spar is flexed.

In another general aspect, a system may include a surgery table comprising a distal end, a proximal end opposing the distal end, and a platform configured to support at least a portion of a patient; a first spar configured to support a first leg of the patient, the first spar comprising a proximal end coupled to the surgery table, a distal end opposing the proximal end of the first spar, a joint disposed between and configured to couple the proximal end to the distal end of the first spar, the joint further configured to rotate and flex the first spar in a generally posterior direction when coupled to the surgery table, an angle between the proximal end and the distal end configured to be reduced as the joint is rotated, and a foot support coupled to and disposed at the distal end of the first spar and configured to secure a foot of the leg of the patient; a second spar configured to support a second leg of the patient, the second spar coupled to the surgery table; and an operable leg drape portion configured to be disposed over at least a portion of the first leg, and a non-operable leg drape portion configured to be disposed over at least a portion of the second leg, the operable leg drape and the non-operable leg drape configured to form a sterile zone between the first leg and the second leg and allow a user to stand between the first leg and the second leg; where the first spar is configured to flex generally in a posterior direction as the leg is flexed generally in an anterior direction opposing the posterior direction such that the leg is able to flex away from the first spar and without the first spar being directly between an upper leg and a lower leg of the leg.

Implementations may include one or more of the following features. System where the operable leg drape portion comprises a leg opening configured to encircle at least a portion of the first leg. System further including a wall drape portion configured to extend between the operable leg portion and the non-operable leg portion to form at least a portion of the sterile zone, where the wall drape portion is configured to extend generally vertically between the operable leg portion and the non-operable leg portion. System where the joint is configured to rotate to flex the first spar to reduce the angle between the proximal end and the distal end of the first spar to an acute angle such that an inferior window is formed proximate to the first spar to allow access to a knee of the first leg generally from an inferior direction. System where the joint is configured to be locked to fix the distal end of the first spar relative to the proximal end of the first spar at a plurality of angles comprising the angle. System where the first spar comprises a straight spar configuration in which the first spar is not pitched or yawed, where the first spar is configured to yaw from the straight spar configuration inward and outward, where the first spar extends generally along a horizontal plane of the surgical table and along an axis generally parallel to a centerline of the surgical table in the straight spar configuration, and where yaw inward and outward of the first spar is relative to the centerline. System where the first spar is configured to yaw outward such that a medial window and a lateral window are formed proximate to the first spar to allow access to the knee of the leg from generally a medial direction and generally a lateral direction. System where the second spar comprises a second straight spar configuration in which the second spar is not pitched or yawed, where the second spar is configured to yaw from the second straight spar configuration inward and outward, where the second spar extends generally along a horizontal plane of the surgical table and along an axis generally parallel to a centerline of the surgical table in the straight spar configuration, where yaw inward and outward of the second spar is relative to the centerline, and where the second spar is configured to yaw outward to form the sterile zone between the first leg and the second leg. System where, as the first spar is flexed, the first spar is configured to translate in a superior direction relative to the surgery table and lower in generally a posterior direction such that a height of a knee of a leg of the patient is generally maintained.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 shows a perspective view of a system for supporting a patient during a surgical procedure according to exemplary embodiments.

FIG. 2 shows a perspective view of a portion of a surgery table according to exemplary embodiments.

FIG. 3 shows a perspective view of a spar according to exemplary embodiments.

FIG. 4 shows an enlarged side view of the spar of FIG. 3.

FIG. 5 shows an enlarged side view of the spar of FIG. 3.

FIG. 6 shows an enlarged perspective view of the spar of FIG. 3.

FIG. 7A shows an enlarged perspective view of the spar of FIG. 3.

FIG. 7B shows an enlarged perspective view of the spar of FIG. 3.

FIG. 8 shows a side cross-section view of the spar of FIG. 3 along line 8-8.

FIG. 9 shows a top view of the system of FIG. 1.

FIG. 10A shows an enlarged perspective view of the spar base of FIG. 10A.

FIG. 10B shows an enlarged perspective view of the spar base of FIG. 10A.

FIG. 11A shows an enlarged perspective view of the spar base of FIG. 10A.

FIG. 11B shows an enlarged perspective view of the spar base of FIG. 10A.

FIG. 12 shows a side cross-section view of the spar base of FIG. 10B along line 12-12.

FIG. 13 shows a perspective view of the spar of FIG. 3 and the spar base of FIG. 10A.

FIG. 14 shows a schematic of the spar base of FIG. 10A.

FIG. 15 shows a schematic of the spar base of FIG. 10A.

FIG. 16 shows a schematic of the spar base of FIG. 10A.

FIG. 17 shows a perspective view of the portion of the surgery table of FIG. 2.

FIG. 18 shows a perspective view of the system of FIG. 1.

FIG. 19 shows a perspective view of the system of FIG. 1.

FIG. 20 shows a perspective view of the system of FIG. 1.

FIG. 21 shows a perspective view of the system of FIG. 1.

FIG. 22 shows a perspective view of the system of FIG. 1.

FIG. 23 shows a perspective view of the system of FIG. 1.

FIG. 24 shows a perspective view of the system of FIG. 1.

FIG. 25 shows a perspective view of the system of FIG. 1.

FIG. 26 shows a perspective view of the system of FIG. 1.

FIG. 27 shows a perspective view of the system of FIG. 1.

FIG. 28 shows a perspective view of the system of FIG. 1.

FIG. 29 shows a perspective view of the system of FIG. 1.

FIG. 30 shows a perspective view of a drape according to exemplary embodiments.

FIG. 31 shows a perspective view of a drape according to exemplary embodiments.

FIG. 32 shows a perspective view of a drape according to exemplary embodiments.

FIG. 33 shows a perspective view of a foot support according to exemplary embodiments.

FIG. 34 shows a top cross-section view of the foot support of FIG. 33 along line 34-34.

FIG. 35 shows a side cross-section view of the foot support of FIG. 33 along line 31-31.

FIG. 36 shows a perspective view of a table module according to exemplary embodiments.

FIG. 37 shows an enlarged perspective of the spar of FIG. 3.

FIG. 38 shows a portion of the top view of the system of FIG. 9.

FIG. 39 shows a perspective view of the foot support of FIG. 33 and the table module of FIG. 36.

FIG. 40 shows an enlarged perspective view of the table module of FIG. 36.

FIG. 41 shows an enlarged perspective view of the spar of FIG. 3.

FIG. 42 shows a top view a UAI offset arm according to exemplary embodiments.

FIG. 43 shows a perspective view the UAI offset arm of FIG. 42.

FIG. 44 shows a perspective view of the portion of the surgery table of FIG. 2 and the spar of FIG. 3.

FIG. 45 shows a rear view of the portion of the surgery table of FIG. 2.

FIG. 46 shows an assembly view of the portion of the surgery table of FIG. 2, a pelvic port adapter and an attachment according to exemplary embodiments.

FIG. 47 shows a perspective view of the pelvic port adapter of FIG. 46.

FIG. 48 shows a perspective view of the attachment for a pelvic port of FIG. 46.

FIG. 49 shows a top view of a knee ledge board pad and a spar pad according to exemplary embodiments.

FIG. 50 shows a perspective view of a trunk pad according to exemplary embodiments.

FIG. 51 shows a perspective view of a lateral flap and a portion of the trunk pad of FIG. 50 according to exemplary embodiments.

FIG. 52 shows a perspective view of a spar board and a spar board pad according to exemplary embodiments.

FIG. 53 shows a side view of the spar board of FIG. 52.

FIG. 54 shows a perspective view of the spar board of FIG. 52.

FIG. 55A shows the spar board of FIG. 52 and the spar board pad of FIG. 52.

FIG. 55B shows the spar board of FIG. 52 and the spar board pad of FIG. 52.

FIG. 56 shows a perspective view of the foot support pad according to exemplary embodiments.

FIG. 57 shows a perspective view of the foot support pad of FIG. 56.

FIG. 58 shows a perspective view of a shoulder cover according to exemplary embodiments.

FIG. 59 shows a perspective view of an elbow cover according to exemplary embodiments.

FIG. 60 shows a perspective view of the elbow cover of FIG. 59.

FIG. 61 shows a perspective view of an end cover according to exemplary embodiments.

FIG. 62 shows an enlarged side view of the portion of the surgery table of FIG. 2.

FIG. 63 shows a perspective view of a pendant according to exemplary embodiments.

FIG. 64 shows a perspective view of a manual lock according to exemplary embodiments.

FIG. 65 shows a perspective view of a head support according to exemplary embodiments.

FIG. 66 shows a perspective view of the head support of FIG. 65.

FIG. 67 shows a perspective view of a spar according to exemplary embodiments.

FIG. 68 shows an enlarged top view of the spar of FIG. 67.

FIG. 69 shows an enlarged perspective view of the spar of FIG. 67.

FIG. 70 shows an enlarged side view of the spar of FIG. 67.

FIG. 71 shows an enlarged perspective view of the spar of FIG. 67.

FIG. 72 shows an enlarged side view of the spar of FIG. 67.

FIG. 73 shows an enlarged perspective view of the spar of FIG. 67.

FIG. 74 shows a perspective view of a foot support according to exemplary embodiments.

FIG. 75 shows a side view of the foot support of FIG. 74.

FIG. 76 shows a side view of the foot support of FIG. 74.

FIG. 77 shows an enlarged perspective view of the foot support of FIG. 74.

FIG. 78 shows a perspective view of a user interface paddle of the foot support of FIG. 74.

FIG. 79 shows a perspective view of the spar of FIG. 67.

FIG. 80 shows a perspective view of the spar of FIG. 67.

FIG. 81 shows a perspective view of traction extension controls according to exemplary embodiments.

FIG. 82 shows a perspective view of a lateral board according to exemplary embodiments.

FIG. 83 shows a perspective view of an imaging board according to exemplary embodiments.

FIG. 84 shows a perspective view of a well leg holder according to exemplary embodiments.

### DETAILED DESCRIPTION

U.S. patent application Ser. No. ______ filed Oct. 27, 2025 titled “SYSTEMS AND METHODS FOR SURGICAL POSITIONING”, U.S. patent application Ser. No. ______ Filed Oct. 27, 2025 titled DRAPE FOR USE IN SURGICAL PROCEDURES, U.S. patent application Ser. No. ______ Filed October 2027 titled ARM SUPPORT FOR SURGICAL PROCEDURES, U.S. patent application Ser. No. ______ Filed Oct. 27, 2025 titled SYSTEMS AND METHODS FOR SURGICAL POSITIONING, and U.S. patent application Ser. No. ______ Filed Oct. 27, 2025 titled SYSTEMS AND METHODS FOR SURGICAL POSITIONING are hereby incorporated by reference in their entirety.

In the following detailed description of embodiments, reference is made to the accompanying drawings, which form a part hereof and in which are shown, by way of illustration, specific embodiments in which the invention may be practiced. Specific details disclosed herein are in every case a non-limiting embodiment representing concrete ways in which the concepts of the invention may be practiced. This serves to teach one skilled in the art to employ the present invention in virtually any appropriately detailed system, structure or manner consistent with those concepts. It will be seen that various changes and alternatives to the specific described embodiments and the details of those embodiments may be made within the scope of the invention. Because many varying and different embodiments may be made within the scope of the inventive concepts herein described and in the specific embodiments herein detailed without departing from the scope of the present invention, it is to be understood that the details herein are to be interpreted as illustrative and not as limiting.

Patient support apparatuses, such as surgery tables, must position and support a patient for the particular procedure being conducted while minimizing or preventing risk to the patient. Systems and methods described herein provide a patient positioning apparatus. The system may include a surgery table that is convertible between a general-purpose surgery table and a spar table configured for orthopedic surgery. For example, the surgery table may be configured for knee surgery. The surgery table may include various attachments to support at least a portion of a patient. Spars, or leg supports, may extend from the distal end of the table to support at least a portion of the legs of the patient. The spars may be foldable, and maneuverable in pitch and yaw directions, and may include one or more joints that may be released to flex, or fold, the spars. A foot support may be coupled to the spar of the operable leg of the patient and may support at least a portion of the foot of the operable leg. The foot support may include a user interface that may allow a user to fold the spar and generally simultaneously translate the spar in the superior direction, the patient's operable leg bending as a function of the respective spar bending. Additional user interfaces may allow control of pitch or yaw of the spar. The spars may be yawed away from one another and may be devoid of distal mechanisms for hip arthroscopy. Therefore, a surgeon may access the knee of the bent operable leg from the inferior and medial directions in addition to the lateral direction, as no intermediate structures limit or prevent access to the knee in the inferior, medial, and lateral directions. As will be discussed, the surgeon may stand in front of the operable knee for direct inferior access and between the spars to access the operable knee directly from the medial or lateral direction. One or more surgical drapes configured for the knee surgery may be draped over the system to allow access to the knee in the inferior, medial, and lateral directions by wrapping around the separable spars. The drapes may also include film, or windows, over user interfaces to allow the user to view user interfaces positioned under the drapes for maneuvering and folding the spars. Reference will now be made to the figures.

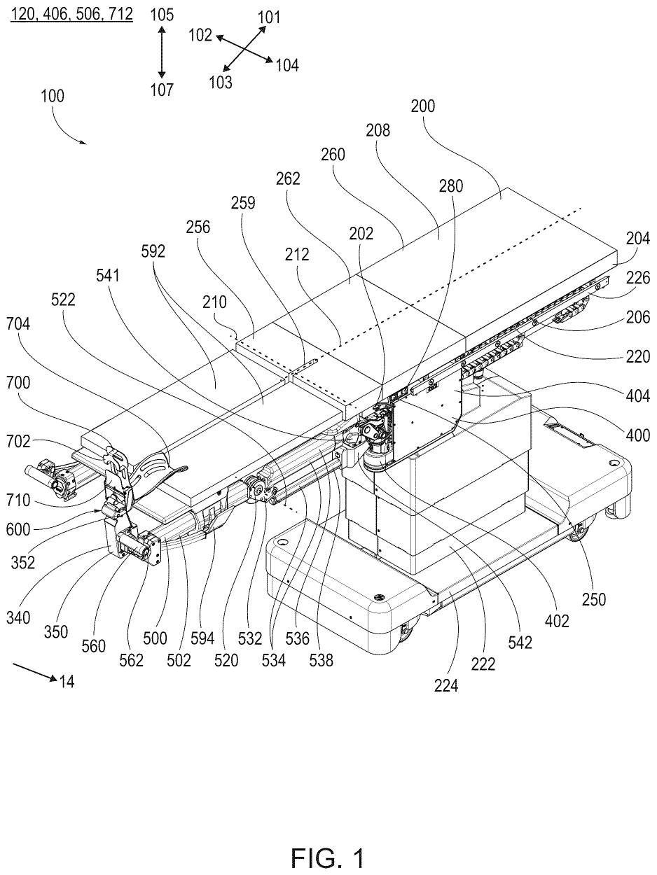

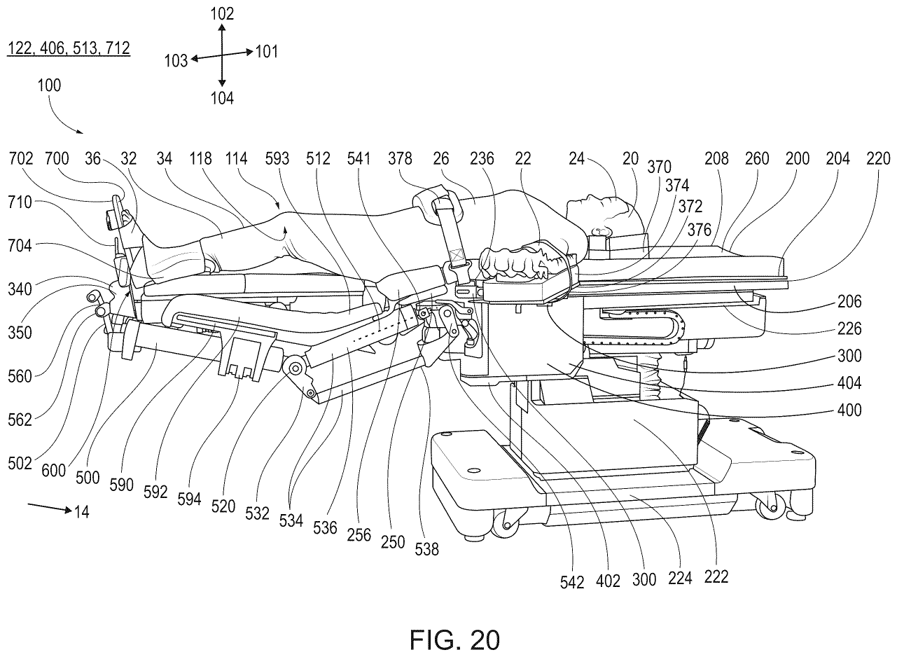

FIG. 1 shows a perspective view of a positioning system 100 for supporting a patient 20 (FIG. 20) for a surgical procedure according to exemplary embodiments. Positioning system 100 may be a modular system in which a surgery table 200 of positioning system 100 may receive one or more removable attachments. Additionally or alternatively, surgery table 200 may include one or more of the attachments when coupled to surgery table 200 such that surgery table 200 may have the functionality of positioning system 100 to support the patient. Additionally or alternatively, each attachment may function separately from positioning system 100, such as with other positioning systems, surgery tables, or apparatuses thereof.

All components of positioning system 100 may be rapidly disinfected. Components may be devoid of or include minimal crevices, openings, and sharp edges to reduce infection risk. As will be discussed, mechanical and software/control interlocks may prevent accidental activation or movement. Individual components may include cutoff switches to negate power and release coupled components for addition fail-safety. Electrical contacts may be covered when not in use, and software may disable power when no component that requires power is attached.

Positioning system 100, surgery table 200, and components thereof may be described with reference to an operable leg 32 (FIG. 20) of patient 20. In one embodiment, operable leg 32 may be a left leg, as shown herein. Accordingly, a medial direction 102 and a lateral direction 104 discussed herein may be with reference to operable leg 32 being a left leg. Additionally or alternatively, operable leg 32 may be a right leg. Positioning system 100, surgery table 200, and components thereof may function with operable leg 32 being a left leg, a right leg, or both legs.

Surgery table 200 may be convertible between a general-purpose surgery table and a specialty surgery table. For example, surgery table 200 may be configured for orthopedic surgeries. As discussed herein, surgery table 200 may be configured for knee surgery. In other embodiments, surgery table 200 may be configured for hip surgery and trauma surgery.

In an embodiment, surgery table 200 may include features that are functional separately and in combination. As shown in the embodiment of FIG. 1, surgery table 200 may include a distal end 202 and a proximal end 204. Distal end 202 may be distal to proximal end 204. In other words, proximal end 204 may oppose distal end 202 and may be superior relative to distal end 202. When patient 20 (FIG. 20) is positioned on surgery table 200, distal end 202 may be closer to legs 32 (FIG. 20) of patient 20 relative to proximal end 204. Distal end 202 may include at least the edge of surgery table 200. Proximal end 204 may include at least the proximal edge of surgery table 200.

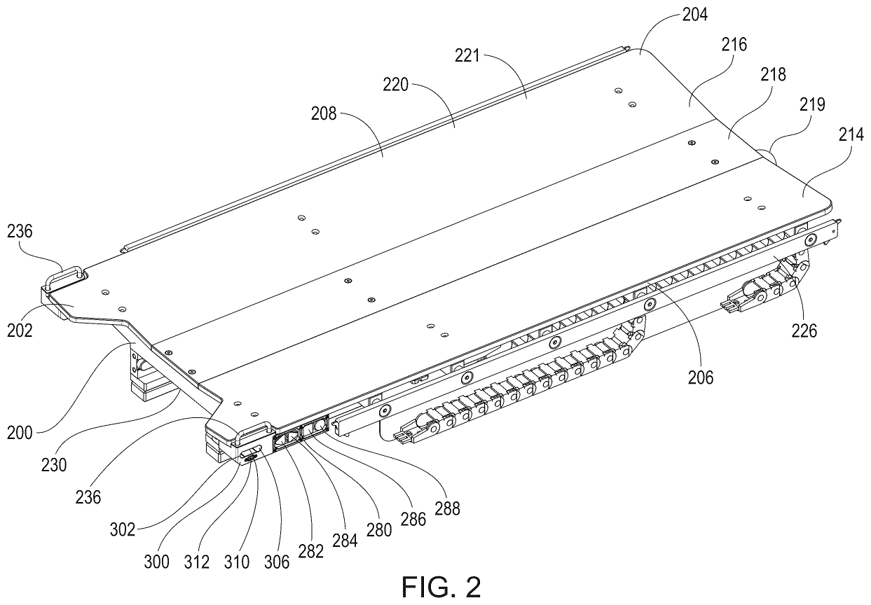

In embodiments, surgery table 200 may also include a platform 220. Platform 220 may support at least a portion of patient 20. For example, platform 220 may support at least the upper body of patient 20 (FIG. 20). A top portion 221 of platform 220, shown in FIG. 2 according to exemplary embodiments, may receive at least a portion of patient 20. Top portion 221 may be generally flat. Alternatively, top portion 221 may include one or more static flaps to form a curved profile that may follow natural curves of patient 20, for example. As shown, top portion 221 may include a first static flap 214, a second static flap 216, and a center static flap 218 intermediate to first static flap 214 and second static flap 216. First static flap 214 may be at an angle 219 relative to center static flap 218. Angle 219 may be between about 0 degrees and about 15 degrees, such as between about 5 degrees and about 10 degrees. Second static flap 216 may similarly be at an angle relative to center static flap 218 such as an angle similar to angle 219. Alternatively, first static flap 214, second static flap 216, and center static flap 218 may form a continuous curved profile. A distance from bottom-most point of center static flap 218 and a top-most point of first static flap 214 and second static flap 216 may be between about half an inch and about three inches, such as about one inch.

Top portion 221 may be generally rigid. Referring again to FIG. 1, top portion 221 may receive one or more pads to support patient 20 (FIG. 20). As shown, a trunk pad 260 may be disposed along platform 220. Trunk pad 260 may support at least a portion of patient 20, such as at least a torso 26 (FIG. 20) of patient 20. Trunk pad 260 will be described further below. Platform 220 may extend from distal end 202 to proximal end 204 such that distal end 202 includes the distal area of platform 220 and proximal end 204 includes the proximal area of platform 220. Platform 220 may also extend from lateral sides of surgery table 200 such that the lateral sides include platform 220. Surgery table 200 may include lateral sides, such as first side 206 and second side 208. First side 206 may oppose second side 208 and may be on opposite sides of a centerline 110 of positioning system 100.

In embodiments, surgery table 200 may include a base 224 that may contact a ground surface 14 of the operating room. Surgery table may include a column 222 coupled to base 224 and platform 220, column 222 being intermediate to platform 220 and base 224. In one embodiment, column 222 may be height adjustable, and may adjust the height of surgery table 200, and therefore positioning system 100, by raising surgery table 200 in an anterior direction 105 or lowering surgery table 200 in a posterior direction 107. In other words, platform 220 may be raised or lowered relative to base 224 between a low position and a high position. Platform 220 may also translate in a superior direction 101 and an inferior direction 103, pitch or Trendelenburg about an axis 210 at distal end 202, and roll or tilt about an axis 212, which may coincide with centerline 110. Base 224 may include a floor lock system to alternately allow or prevent motion of surgery table 200 and/or a drive assist user interface module to permit an operator of surgery table 200 to selectively control movement of surgery table 200, as disclosed in U.S. patent application Ser. No. 15/610,486, the disclosure of which is hereby incorporated by reference in its entirety.

An embodiment may include a spar base 400 is shown in FIG. 1. In an embodiment, spar base 400 may be part of positioning system 100. In another embodiment, spar base 400 may be a part of surgery table 200. In another embodiment, spar base 400 may function separately, such as with other positioning systems, surgery tables, or apparatuses thereof. In an example, spar base 400 may include features that are functional separately and in combination.

As shown in the embodiment of FIG. 1, spar base 400 may be positioned beneath platform 220. Spar base 400 may be positioned beneath platform 220 at distal end 202. Surgery table 200 may include a rail 226 along first side 206 and second side 208 for receiving various accessories having a corresponding rail clamp, including spar base 400. Spar bases 400 may be coupled to surgery table 200 along at least one of first side 206 and second side 208, such as both first side 206 and second side 208. Spar bases 400 may be disposed below rails 226.

Spars 500 are also shown in the embodiment of FIG. 1. In an example, spar 500 may be part of positioning system 100. In another example, spar 500 may be a part of surgery table 200. In another example, spar 500 may function separately, such as with other positioning systems, surgery tables, or apparatuses thereof. In an example, spar 500 may include features that are functional separately and in combination.

One or more spars 500 may extend from spar base 400, such as from a distal end 402 of spar base 400. Each spar 500 may support at least one leg 32 (FIG. 20) of patient 20 (FIG. 20). Spar base 400 may include a distal end 402 and a proximal end 404. Distal end 402 may be distal to proximal end 404. In other words, proximal end 404 may oppose distal end 402 and may be superior relative to distal end 402. Distal end 402 may be at distal end 202 of surgery table 200 such that spar 500 may extend from distal end 202 of surgery table 200. Spar base 400 may translate a distance along surgery table 200 between distal end 202 and proximal end 204 of surgery table 200. Accordingly, spar 500 may translate as spar base 400 translates because spars 500 extend from spar base 400. Spar base 400 may translate along surgery table 200 such that it translates relative to platform 220, column 222, and base 224. Spar base 400 may translate in superior direction 101 such that spar 500 may extend from a point superior to distal end 202 of surgery table 200. Spar base 400 may translate in inferior direction 103 after translating in superior direction 101 to be positioned at distal end 202 of surgery table 200.

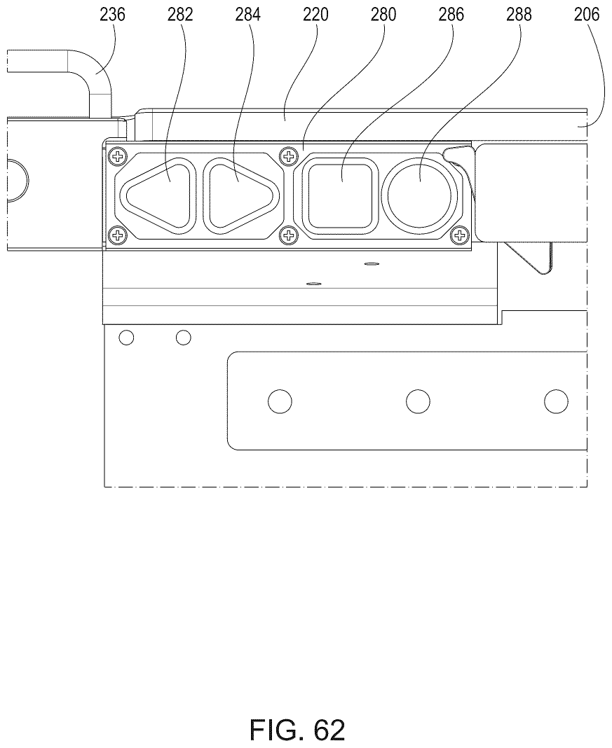

Spar base 400 may be electromechanically translated. Spar base 400 may include an electromechanical driver. In certain embodiments, electomechanical includes lead screw 272 as discussed herein. Additionally or alternatively, another part of surgery table 200 may include an electromechanical driver coupled to spar base 400. The electromechanical driver may translate spar base 400 a distance along surgery table 200 between distal end 202 and proximal end 204 of surgery table 200. One or more controls may control the electromagnetic driver, such as one or more traction controls 280 (FIG. 62), which will be discussed further below.

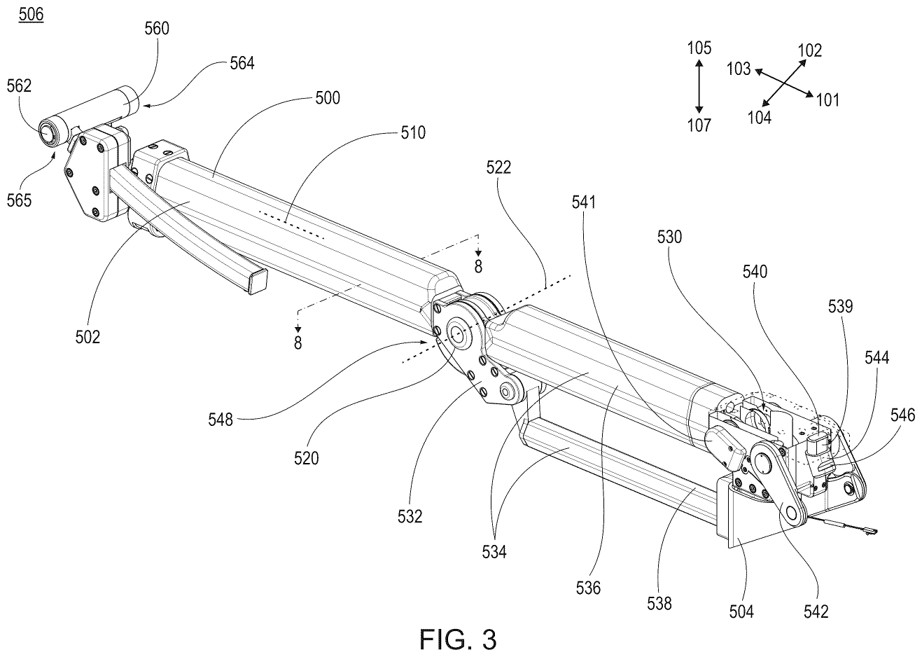

Spar 500 is shown in FIGS. 3-8 according to exemplary embodiments. With reference to FIG. 3, spar 500 may include a distal end 502 and a proximal end 504. Distal end 502 may be distal to proximal end 504. In other words, proximal end 504 may oppose distal end 502 and may be superior relative to distal end 502. In one embodiment, distal end 502 may be length-adjustable. For example, distal end 502 may include a telescoping portion that is retractable to reduce the length of spar 500 and extendable to increase the length of spar 500. In this way, spar 500 may be adapted for different sized patients. Distal end 502 of spar 500 may receive two or more table modules 340 (FIG. 1), which may interface with surgery table 200 such as by drawing power from surgery table 200 and inheriting the articulation of spar 500. Table modules 340 may include a foot support module 350, which may receive one or more foot supports, such as a foot support 700 to support at least one foot 36 (FIG. 20) of patient 20 (FIG. 20). Table modules 340, foot support module 350, and foot support 700 will be described further below.

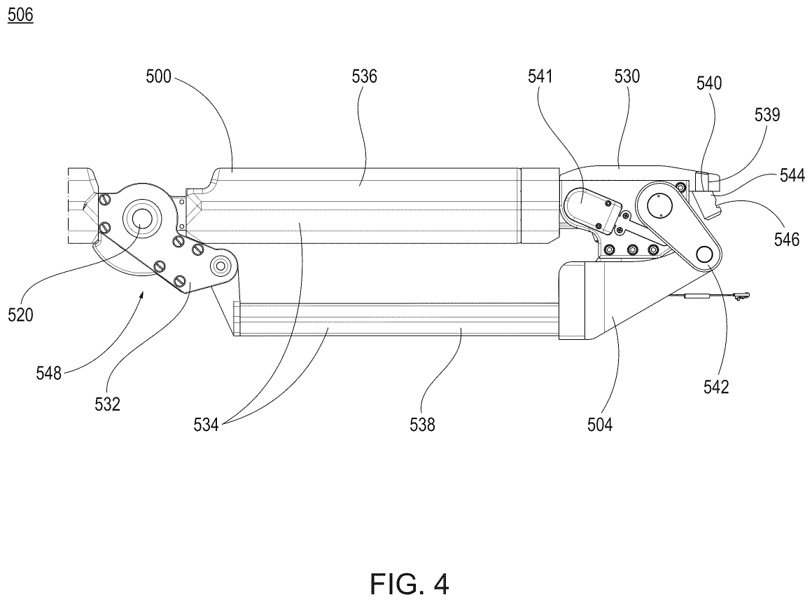

In embodiments, distal end 502 of spar 500 may be coupled to proximal end 504 by a joint 520. Joint 520 may be alternately locked and releasable to allow spar 500 to fold, or flex, by rotation about a joint axis 522 of joint 520. As shown in FIGS. 3-4, spar 500 may be in a straight spar configuration 506. In straight spar configuration 506, spar 500 may be generally straight, e.g., distal end 502 and proximal end 504 may be generally aligned along the same plane as spar 500 may extend along a horizontal spar axis 510 extending along spar 500 when spar 500 is in straight spar configuration 506. From straight spar configuration 506, spar 500 may pitch such that spar 500 is angled up in anterior direction 105 or angled down in posterior direction 107. Spar 500 may also move in a yaw direction, such as in medial direction 102 or lateral direction 104. Proximal end of spar 504 may be coupled to surgery table 200, such as to spar base 400, such that distal end 502 of spar 500 pitches and yaws relative to proximal end of spar 504.

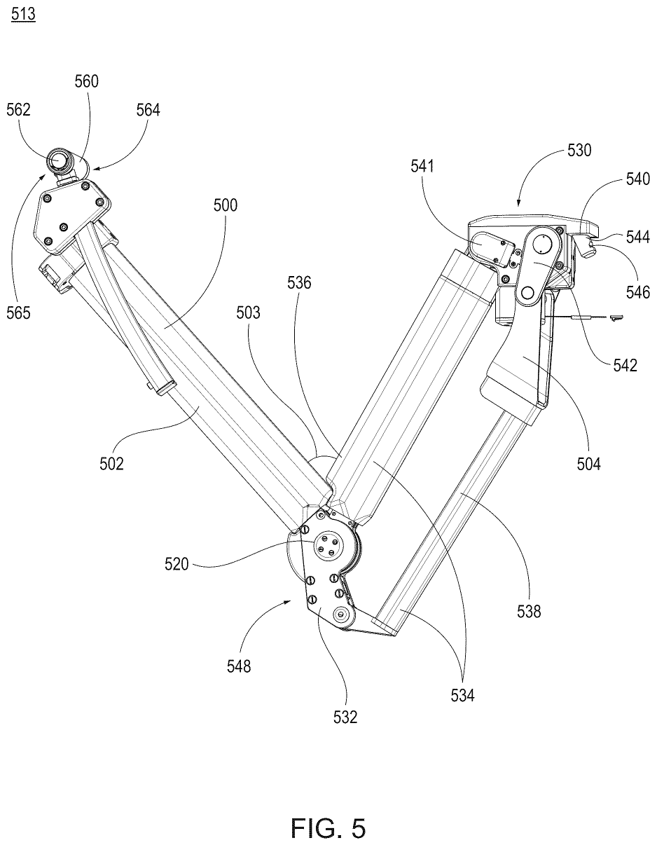

In embodiments, in addition to pitch and yaw, spar 500 may flex such that distal end 502 folds toward proximal end 504, as shown in FIG. 5. Spar 500 may thus be in a folded spar configuration 513. In folded spar configuration 513, spar 500 may be at least partially folded, or flexed. In other words, in folded spar configuration 513, spar 500 may be folded, or flexed, at least partially or fully along a range of motion of spar 500 flex. When folding toward proximal end 504, distal end 502 may raise or lower relative to joint 520. Joint 520 may be released to raise or lower distal end 502 such that distal end 502 may be at an angle of up to and about 70 degrees relative to proximal end 504. Spar 500 may be fully flexed such that distal end 502 may be about 70 degrees from proximal end 504. Spar 500 may be at least partially flexed such that distal end 502 is between about 185 degrees and about 75 degrees, such as between about 150 degrees and about 90 degrees.

When joint 520 is released, spar 500 may move between straight spar configuration 506 shown in FIGS. 3-4 and folded spar configuration 513, one folded spar configuration 513 shown in FIG. 5. Spar 500 may fold less or more than as shown in FIG. 5. As shown, an angle 503 between distal end 502 and proximal end 504 may be manipulated as spar 500 is folded. Joint 520 may include angle 503 between distal end 502 and proximal end 504. Angle 503 may be about 180 degrees when spar 500 is in straight spar configuration 506. Angle 503 may be reduced as joint 520 is rotated. Angle 503 may be reduced to between about 30 degrees and about 150 degrees, such as between about 60 degrees and about 120 degrees, such as between about 70 degrees and about 90 degrees, such as about 90 degrees. Spar 500 may be easily movable. For example, spar 500 may be lightweight. Spar 500 may include minimal attachments to be lightweight. For example, distal end 502 of spar 500 may not include a traction mechanism such that spar 500 is lightweight and distal end 502 of spar is directly accessible from inferior direction 103.

In embodiments, joint 520 may be locked in a particular orientation when a corresponding user interface is released. Accordingly, spar 500 may be stiff, or generally immovable, when joint 520 is locked. For example, distal end 502 of spar 500 may include a handle 560 having one or more joint controls, such as a control 562, a control 564, and a control 565. At least one of control 562, control 564, and control 565 may include joint controls. Control 562, control 564, and control 565 may each include a button actuator or another actuation mechanism, such as a switch, lever, or slider, for example. In certain embodiments, the operation of joint controls from handle 560 may depend on whether a table module 340 is coupled to spar 500 at distal end 502, and the operation of joint controls from handle 560 may change depending on which table module 340 is attached. For example, foot support module 350 may allow for handle 560 to flex spar 500 by articulating joint 520. However, another table module 340 coupled to spar 500 may prevent handle 560 from operating to flex spar 500. Accordingly, joint 520 may be released, or unlocked, via one or more joint controls remote from joint 520.

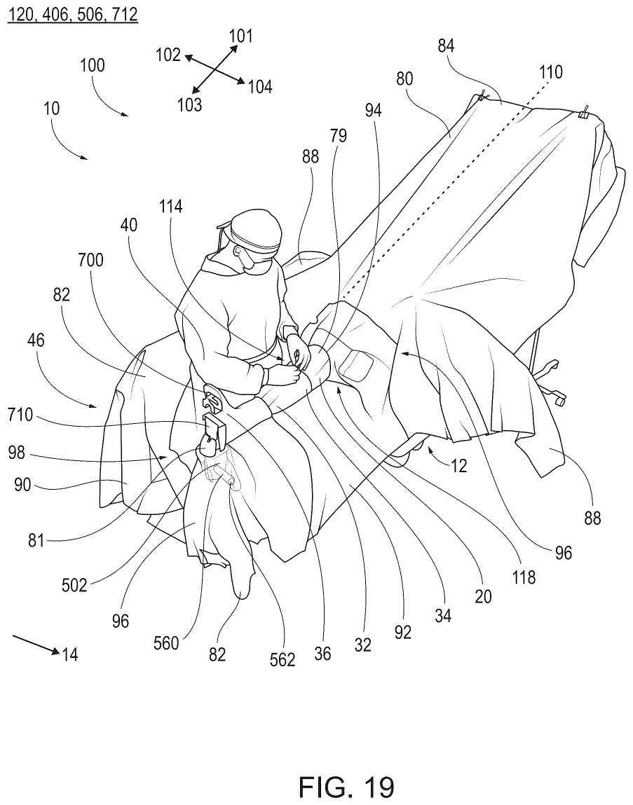

Instead of control 562, control 564, and control 565, and other controls integrated into a component of positioning system 10 (FIG. 1), a user 40 (FIG. 19) may have access to portable controls, or a relocatable user interface (not shown). Such portable controls may be distinct from a pendant, and may a wireless housing. The wireless housing may be flexibly placed on any component of positioning system 10, such as spar 500, one or more drapes, handle 560, or surgery table 200 (FIG. 1), for example. The wireless housing may be a wireless controller. The wireless housing may include tactile physical buttons and/or touch screens. Controls on the wireless housing may include control 562, control 564, and control 565, and other controls described herein. Controls on the wireless housing may be user-selectable and programmable to allow each user 40 to select control placement and functions according to personal preference.

Proximal end 504 may also include one or more links, bars, or arms, such as links 534, a passive link 532, a stationary link 541, and a rotating link 542. Joint 520 may be coupled to passive link 532, which may be coupled to one or more gears 530 via one or more links 534. One or more links 534 may include a first link 536 and a second link 538. First link 536 and second link 538 may be parallel links. As shown in FIGS. 3-5, first link 536 and second link 538 may be parallel in straight spar configuration 506 and folded spar configuration 613. In other words, first link 536 and second link 538 may be parallel through the range of motion of spar 500. The orientation of at least one of first link 536 and second link 538 may be controlled by one or more gears 530. For example, one or more gears 530 may control the orientation of first link 536 to control the orientation of proximal end 504. First link 536 may be coupled to stationary link 541. Second link 538 may be coupled to rotating link 542. Stationary link 541 and rotating link 542 may be coupled to one or more gears 530 to control the orientation of proximal end 504. As proximal end 504 is folded, a 4-bar linkage is formed with at least rotating link 542, passive link 532, first link 536, and second link 538 rotating around passive link 532. The orientation control of at least one of first link 536 and second link 538 will be discussed further below. Those skilled in the art understand that a 4-bar (or four-bar) linkage is considered to be a type of movable closed chain linkage. It consists of four bodies, called bars or links, connected in a loop by four joints. Generally, in some embodiments, the joints are configured so the links move in parallel planes.

As spar 500 flexes, proximal end 504 may fold toward distal end 502. Referring to FIG. 5, in certain embodiments, proximal end 504 may include one or more gears 530 that may control the orientation of proximal end 504. For example, one or more gears 530 may include two gears 530. Gears 530 may be arranged as a pair. Gears 530 may counterrotate relative to one another and mesh together. A first gear 530 of the pair may include a shaft coupled to stationary link 541 to control the orientation of stationary link 541. A second gear 530 of the pair may include a shaft coupled to rotating link 542 to control the orientation of rotating link 542. Stationary link 541 may be coupled to first link 536 of spar 500. As the first gear 530 rotates, stationary link 541 may remain generally stationary, and link 536 may rotate around stationary link 541. Rotating link 542 may be coupled to second link 538 of spar 500 by gears 530. As the second gear 530 rotates, link 542 may be permitted to rotate by the intermeshing gears 530, and link 548 may rotate relative to link 542. Gears 530 counterrotating and meshing may cause rotating link 542 to rotate at substantially the same time as gears 530 rotate. Rotating link 542 may rotate towards stationary link 541, as shown in FIG. 5. Accordingly, link 536 and link 538 may be moved such that link 536 and link 538 remain parallel to one another. Passive link 532 may rotate in inferior direction 103 (FIG. 1) to compensate for rotating link 542 rotating toward stationary link 541. Rotating link 542 may be rotated away from stationary link 541 and passive link 532 may rotate in superior direction 105 (FIG. 1) as spar 500 is unfolded at least partially, such as unfolded fully, to reach straight spar configuration 506 (FIG. 3). Accordingly, in embodiments, the pitch of distal end 502 may be controlled by rotation of passive link 532, which is linked via gears 530 to rotation of proximal end 504.

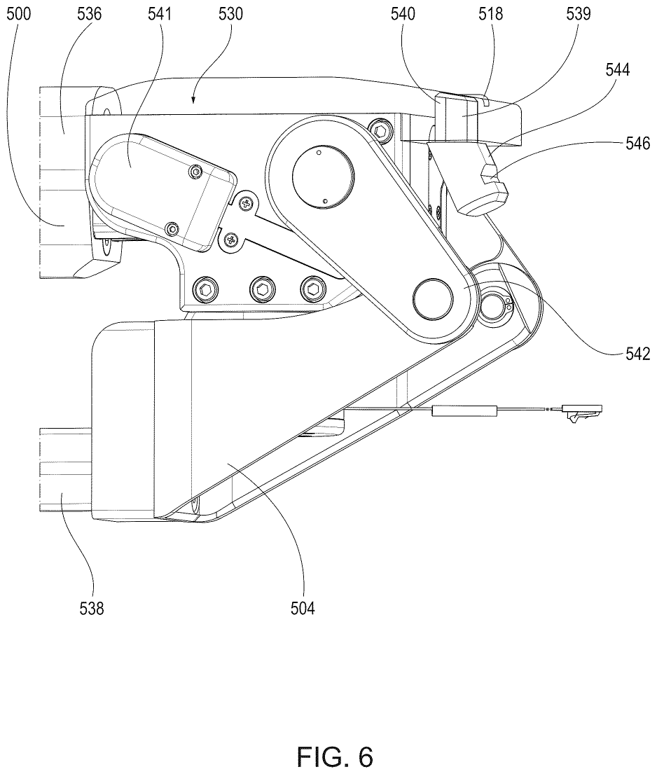

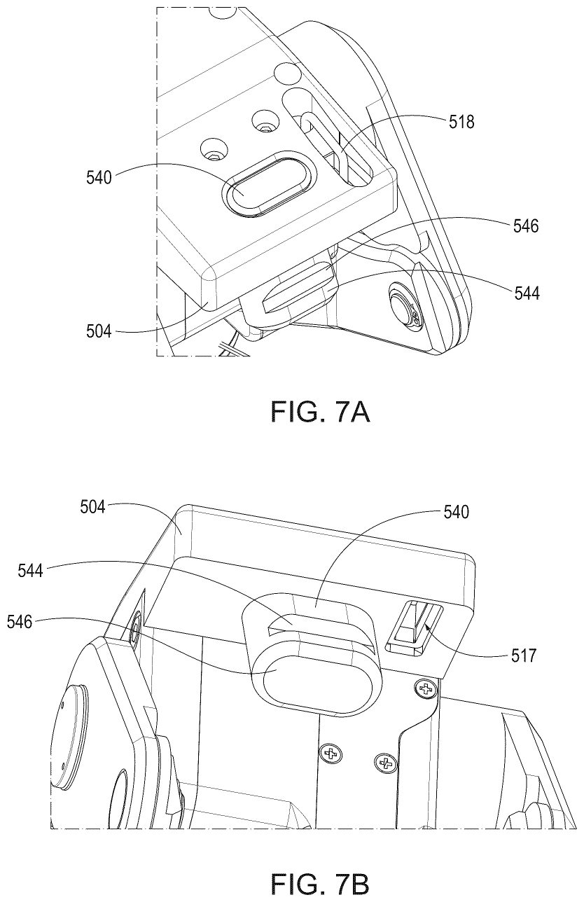

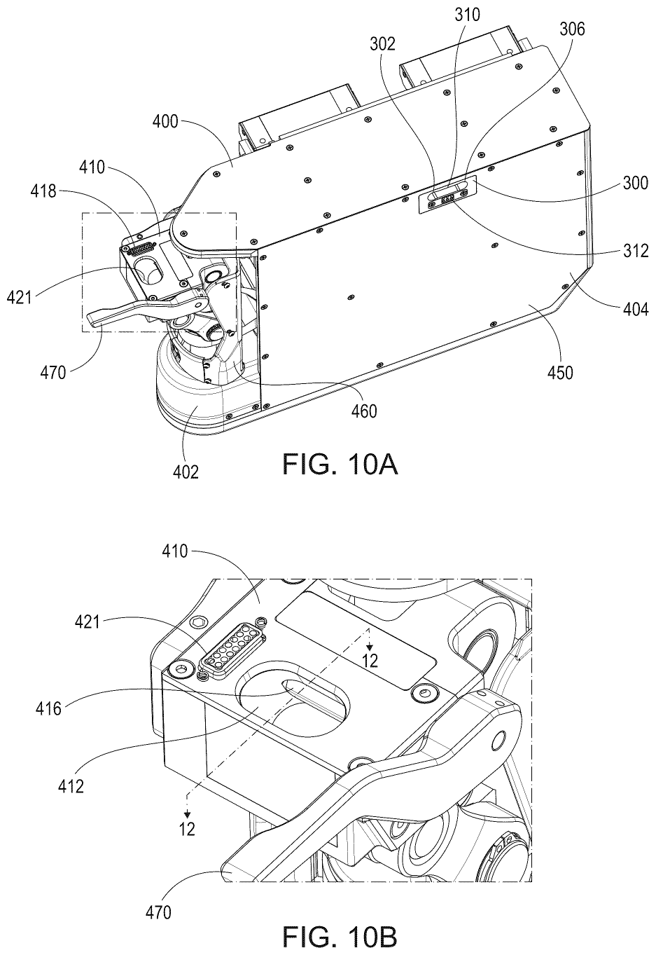

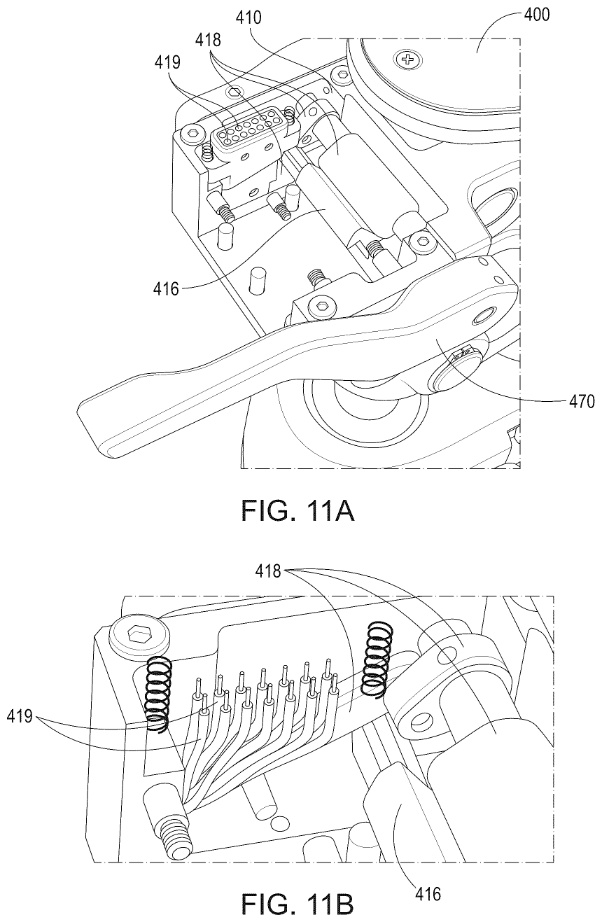

In embodiments, proximal end 504 of spar 500 may couple to spar base 400 (FIG. 10A). With reference to FIG. 6, proximal end 504 of spar 500 may include a projection 540, which may extend outwardly from proximal end 504. Projection 540 may include an inclined face 544 and a notch 546. Notch 546 may receive a boss 416 (FIG. 10B) inside of spar base 400. Projection 540 may be received by spar base 400 to couple spar 500 to spar base 400. In embodiments, spar 500 may include a pin 518 and/or an electrical receptacle 517 as shown with reference to FIGS. 7A-B. Pin 518 and electrical receptacle 517 may couple to an electrical connection 418 (FIG. 11B) of spar base 400 to allow spar 500 to receive power from, and communicate with, positioning system 100 and components thereof.

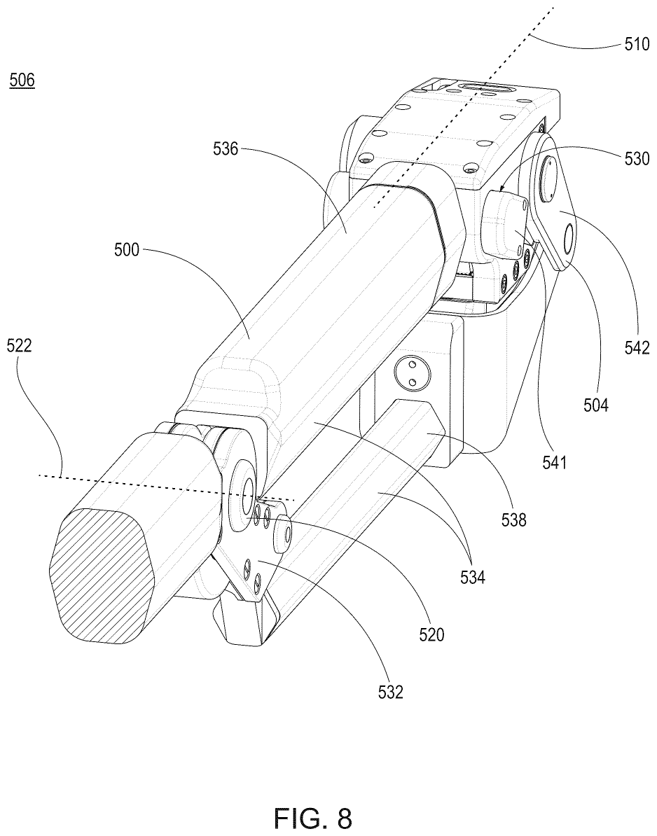

With reference to FIG. 8, in certain embodiments, spar 500 may include one or more profiles that may enhance radiolucency and/or radio-uniformity. As shown, spar 500 may include a cross-section having a substantially hexagonal shape. Accordingly, material may be minimized toward edges of spar 500 to enhance radiolucency. In other words, the angled walls provided by the substantially hexagonal shape minimize the x ray path length and reduce attenuation lines in x ray images. In addition, edges of spar 500 may be rounded rather than sharp. Including curves rather than sharp edges may enhance to enhance radiolucency and/or radio-uniformity.

Spar 500 may include a portion that is substantially radiolucent. Alternatively, spar 500 may be mostly or entirely radiolucent. For example, spar 500 may consist essentially of carbon fiber. In other words, spar 500 may be composed mostly or entirely of carbon fiber. In this way, spar 500 may be radiolucent when it is composed of radiolucent carbon fiber material. In certain embodiments, proximal end 504 of spar 500 may be at least partially non-radiolucent by including metal. For example, one or more gears 530 at proximal end 504 may include metal, such as steel. Accordingly, one or more gears 530 may not be radiolucent. Spar 500 may be translated in superior direction 101 to move at least a portion of proximal end 504 superior to and outside of any imaging window to avoid metal of proximal end 504 from being within the imaging window. Remaining portions of spar 500 may be substantially radiolucent. One or more links 534 passive link 532, joint 520, first link 536, and second link 538 may be include a portion that is substantially radiolucent. Alternatively, one or more links 534 passive link 532, joint 520, first link 536, and second link 538 may be mostly or entirely radiolucent. For example, one or more links 534 passive link 532, joint 520, first link 536, and second link 538 may consist essentially of carbon fiber. In other words, these components may be composed mostly or entirely of carbon fiber. In this way, these components may be radiolucent.

In still further embodiments, spar 500 may be substantially hollow. Spar 500 may include wires 570 (not shown) to connect spar 500 from spar base 400 (FIG. 10A) at electrical connection 418 (FIG. 11B) to one or more controls, such as controls of handle 560, e.g., control 562, control 564, and control 565, and manual lock 548. Wires 570 may also connect spar 500 to UAI port 300 at distal end 502 of spar 500. Wires 570 may be made substantially or completely of carbon fiber. Alternatively, wires 570 may be made substantially or completely of copper, or another metal. The metal of wires 570 may be located inside spar 500 adjacent to an outside edge of spar 500, and therefore may not materially affect the translucency of spar 500. Wires 570 may conduct electricity and/or an electrical signal. In still further embodiments, spar 500 may be substantially or entirely made of carbon fiber between distal end 502 where table modules 340, such as foot support module 350 (FIG. 1), are mounted and proximal ends of first link 536 and second link 538.

In embodiments, spar base 400 (FIG. 10A) may be radiolucent like spar 500. Alternatively, spar base 400 may include metal. For example, components of spar base 400 may include steel. In other embodiments, spar base 400 is not radiolucent.

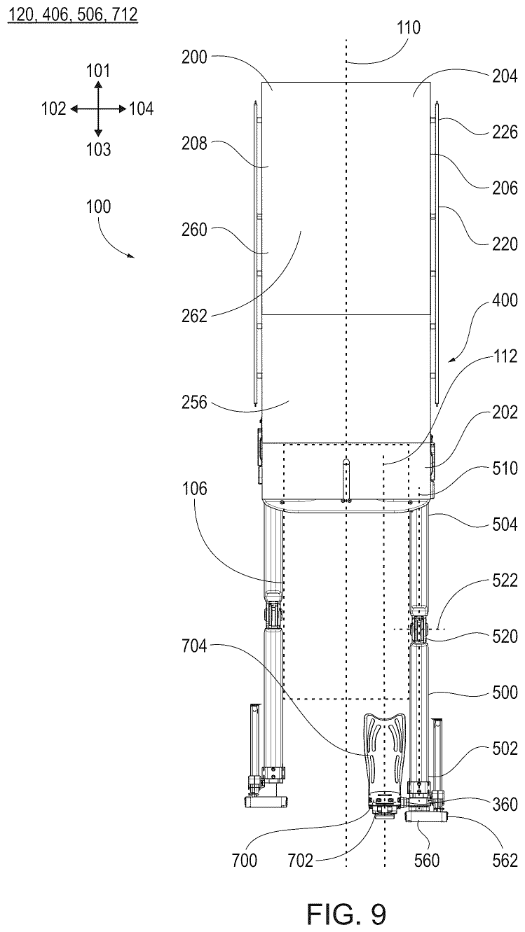

Imaging of patient 20 (FIG. 20) may be conducted from a generally top-down view in the posterior direction 107 (FIG. 1), shown with reference to FIG. 9 according to exemplary embodiments. A top-down lower body imaging window 106 is shown in FIG. 9. Surgery table 200 may include top-down lower body imaging window 106 to facilitate x ray and other imaging for orthopedic surgeries. positioning system 100 (FIG. 1) may also facilitate side imaging and oblique imaging.

In this embodiment, spar base 400, which may include metal, may be superior to and outside of top-down imaging window 106. Spar base 400 may extend to distal end 202 of surgery table 202 and imaging window 106 may extend from distal end 202. Therefore, in embodiments, spar base 400 may be excluded from imaging window 106. Spar base 400 may be translated further superior such that spar 500 coupled to spar base 400 is at least partially superior to distal end 202 of surgery table 200. In this way, any metal at proximal end 504 of spar 500 may be superior to and outside of top-down imaging window 106.

In embodiments, the entirety of spar 500 may also be excluded from top-down imaging window 106. As discussed, spar 500 may extend from spar base 400. As spar base 400 may be disposed along a lateral edge of surgery table 200, such as first side 206 or second side 208, spar 500 may extend from a lateral edge of surgery table 200. As shown, spar 500 may extend from a lateral edge of surgery table 200 along horizontal spar axis 510. Leg 32 (FIG. 20) of patient 20 (FIG. 20) may extend away from spar 500 to foot support 700. Foot support module 350 may be mounted medial to the spar 500, and extend outwardly from spar 500 in medial direction 102 such that such that spar 500 is laterally offset from foot support module 350, and therefore at least a portion of leg 32 of patient 20. Thus, horizontal spar axis 510 may be lateral to a limb axis 112, which may extend along leg 32. In other words, leg 32 may be interior to spar 500. In embodiments, top-down imaging window 106 may surround leg 32 but not spar 500. Accordingly, leg 32 may be within top-down imaging window 106 while spar 500 may be laterally offset from top-down imaging window 106. Alternatively, spar 500 may be at least partially, or fully, included in top-down imaging window 106. As discussed, spar 500 may be radiolucent such that spar 500 does not inhibit imaging if at least partially, or fully, included in top-down imaging window 106.

Spar base 400 is further shown in FIGS. 10A-13 according to exemplary embodiments. With reference to FIGS. 10A-12, spar base 400 may include a spar mount 410 to receive spar 500 (FIG. 3). Spar mount 410 may include a receptacle 412 having an inclined wall 414. Accordingly, spar mount 410 may receive projection 540 (FIG. 4) of spar 500 as receptacle 412 may have inclined wall 414 to receive inclined face 544 (FIG. 4) of projection 540. Receptacle 412 may also include an electrical connection 421 and a boss 416. Boss 416 may be a projection that is retractable to alternately actuate and release electrical connection between spar base 400 and spar 500. Boss 416 may project into notch 546 (FIG. 4) such that electrical connection 421 of spar base 400 is coupled to pin 518 (FIG. 7A) through electrical receptacle 518 (FIG. 7B) of spar 500. For example, electrical receptacle 518 may be an opening that may receive electrical connection 421, which may include one or more electrical connector pins 419, shown in FIGS. 11A-B. Boss 416 may couple to notch 546 when spar 500 is coupled to spar base 400 at distal end 402 of spar base 400.

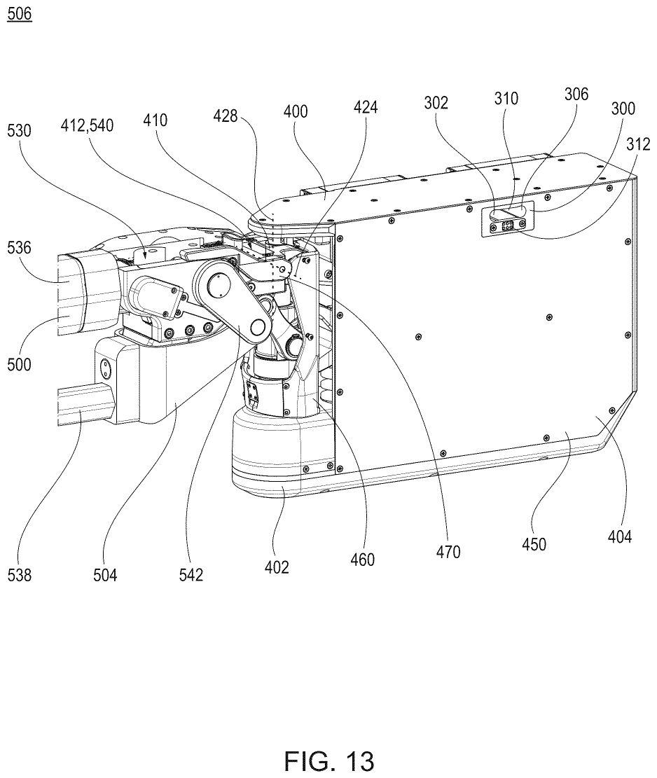

Spar 500 coupled to spar base 400 by spar mount 410 receiving projection 540 is shown in FIG. 13. A handle 470 may be pivoted up to unlock spar 500 and allow spar 500 to be removed from spar base 400, and pivoted down to lock spar 500 into spar base 400. Handle 470 may also be pivoted to alternately actuate and release the electrical connection between spar 500 and spar base 400. Another mechanism for locking spar 500 to spar base 400 may be used, such as a button, switch, or other control.

Handle 470 shown in FIG. 13 may be in a pivoted down position to lock spar 500 (FIG. 3) and actuate the electrical connection between spar 500 and spar base 400. Referring again to FIGS. 11A-B, handle 470 may be pivoted down, causing electrical connection 421 to elevate. Boss 416 may be a projection that is shaped such that it is retractable as handle 470 rotates upward. Pivoting handle 470 down such that boss 416 engages notch 546 and electrical connection 421 is raised and coupled to pin 518 (FIG. 7A) and electrical receptacle 517 (FIG. 7B) of spar 500 (FIG. 3) may rotate a series of linkages 418. Linkages 418 may raise pins 419 inside of electrical connection 421 in order to provide an electrical connection through spar base 400 to spar 500.

Retracting boss 416 may also retract electrical connection 418. Rotating handle 470 upward such that boss 416 and electrical connection 421 are retracted may rotate linkages 418 such that pins 419 are lowered and the electrical connection through spar base 400 is disconnected. Boss 416 may be retracted from notch 546 of spar 500 and spar 500 may be removed from spar base 400.

With reference to FIGS. 3-4 and 12-13, as projection 540 of spar 500 is inserted into receptacle 412, projection 540 may both be inserted and moved in superior direction 101 (FIG. 1) inside of receptacle 412 by nature of inclined face 544 of projection 540 and inclined wall 414 of spar base 400. Electrical connection 421 and pins 419 may be lowered during insertion such that electrical connection 421 and pins 419 are clear from any shear induced by spar 500, such as projection 540, as spar 500, such as projection 540 are moved in superior direction 101 during insertion. Only when spar 500 and projection 540 are seated, electrical connection 421 and pins 419 may be raised vertically in anterior direction 105 (FIG. 1) into connection with pin 518 (FIG. 7A) and electrical receptacle 517 (FIG. 7B).

Referring to FIGS. 3-13, pin 518, electrical receptacle 517, projection 540, pins 419, and spar mount 410 may be generally at an angle at about which spar 500 is installed, e.g., generally an angle of inclined face 544 of projection 540 of spar 500. In this way, components of spar 500 and spar base 400 may be nested during coupling of spar 500 and spar base 400.

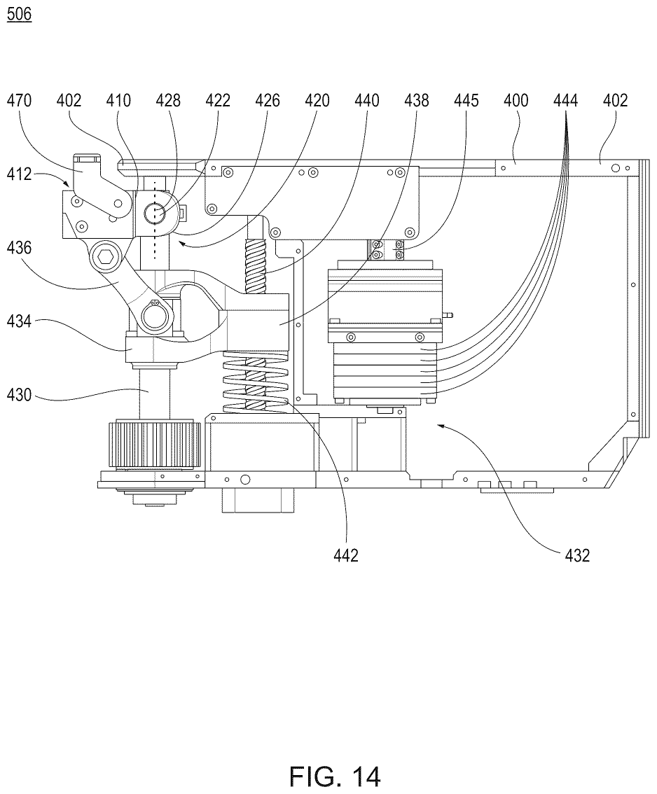

A housing face 450 and a pivot post housing 460 are removed to show additional spar base 400 components as shown in FIG. 14 according to exemplary embodiments. As discussed, spar 500 may be maneuvered in pitch and yaw directions. In embodiments, spar base 400 may include one or more joints 420 to articulate spar mount 410. In further embodiments, the one or more joints 420 may include metal. Spar base 400 may be excluded from top-down imaging window 106 (FIG. 9) such that components of spar base 400 may not affect imaging.

In certain embodiments, spar base 400 may include a pivot post 430 as shown in FIG. 14. Pivot post 430 may extend from distal end 402 of spar base 400. One or more joints 420 may include a first joint 422 to articulate spar mount 410 in a pitch direction such that first joint 422 may be a pitch joint. First joint 422 may be rotatably coupled to pivot post 430. For example, spar mount 410 may be rotatably coupled to pivot post 430 and may include first joint 422. First joint 422 may cause rotation of spar mount 410 about a first rotational axis 424 (FIG. 13), which may be a pitch axis. One or more joints 420 may also include a second joint 426 such that second joint 426 may be a yaw joint. Second joint 426 may be rotatably coupled to pivot post 430. For example, spar mount 410 may be rotatably coupled to pivot post 430 and may include second joint 426. First joint 422 may cause rotation of spar mount 410 about a second rotational axis 428, which may be a yaw axis. As shown in FIG. 13, first rotation axis 424 for first joint 422 and second rotation axis 428 for second joint 426 may intersect.

Referring to FIGS. 13-14, in another embodiment, first rotation axis 424 (FIG. 13) for first joint 422 and second rotation axis 428 for second joint 426 may intersect. In other words the pitch and yaw axes of spar 500 may intersect. First rotation axis 424 for first joint 422 and second rotation axis 428 for second joint 426 may intersect superior to an imaging window, such as top-down imaging window 106 (FIG. 9). In this way, one or more joints 420 are superior to and outside of an imaging window. In addition, first rotation axis 424 for first joint 422 and second rotation axis 428 for second joint 426 may translate as spar base 400 translates a distance along surgery table 200 (FIG. 1) between distal end 202 (FIG. 1) and proximal end 204 (FIG. 1) of surgery table 200 (FIG. 1). Accordingly, spar 500 may translate as spar base 400 translates because spar 500 extends from spar base 400, and spar base 400 also includes the pitch and yaw joints 420 and axes for spar 500.

Spar base 400 may also assist spar 500 in pitch motion. In other words, spar base 400 may provide gravity compensation to lift and lower spar 500 in anterior direction 105 (FIG. 1) and posterior direction 107 (FIG. 1), respectively, without user 40 (FIG. 19) having to lift or lower the full weight of spar 500, in other words “lift assist.” In another embodiment, spar base 400 includes the pitch and yaw axis for spar 500, translates spar 500 and includes lift assist for spar 500.

A lift assist mechanism 432 of spar base 400 is shown in FIGS. 14-16 according to exemplary embodiments. Lift assist mechanism 432 may include a combination of linear and rotation spring assist to provide lift assist to spar 500. Linear motion along pivot post 430 may transfer the moment load to rotational motion via a back-driven ball nut 438 and screw 440, which will be described further below. The rotational motion may travel through a gearbox 446, which will be described further below, into an input shaft of a lift assist and brake assembly. In one embodiment, the total mechanical advantage from ball nut 438 and screw 440 and gearbox 446 includes a range from about 60:1 to 100:1, such as about 70:1 to about 90:1, such as about 80:1.

In an embodiment, lift assist mechanism 432 may be part of positioning system 100. In another example, lift assist mechanism 432 may be a part of surgery table 200. In another example, lift assist mechanism 432 may be a part of spar base 400. In another example, lift assist mechanism 432 may function separately, such as with other positioning systems, surgery tables, or apparatuses thereof. In an example, lift assist mechanism 432 may include features that are functional separately and in combination.

FIG. 14 shows lift assist mechanism 432 when spar 500 (FIG. 3) is in straight spar configuration 506. Accordingly, spar 500 is not being lifted or lowered and instead is horizontal relative to ground surface 14 (FIG. 1). In straight spar configuration 506, a linkage 436 of lift assist mechanism 432 is not actuating spar mount 410. Thus, spar mount 410 appears generally horizontal relative to ground surface 14. Lift assist mechanism 432 may include a slider 434 to actuate spar mount 410 such that spar mount 410 is non-horizontal relative to ground surface 14. Slider 434 may translate along pivot post 430 to actuate spar mount 410. Either directly or operatively, slider 434 may be coupled to spar mount 410 such that actuating slider 434 may actuate spar mount 410. For example, slider 434 may be coupled to spar mount 310 by a linkage 436. Thus, linkage 436 may be actuated by slider 434 to actuate spar mount 410.

In one embodiment, lift assist mechanism 432 may also include ball nut 438 and screw 440. Slider 434 may be coupled to ball nut 438, which may translate along screw 440, such as in anterior direction 105 (FIG. 1) and posterior direction 107 (FIG. 1). In one embodiment, as shown in FIG. 11, lift assist mechanism 432 may include a first spring 442. In one embodiment, first spring 442 may be a coil spring. In addition, first spring 442 may be a die spring and may be coupled to screw 440. Accordingly, like ball nut 438, first spring 442 may move relative to screw 440. First spring 442 may also be coupled to ball nut 438. Accordingly, ball nut 438 may translate in anterior direction 105 (FIG. 1) or posterior direction 107 (FIG. 1) to respectively expand or compress first spring 442.

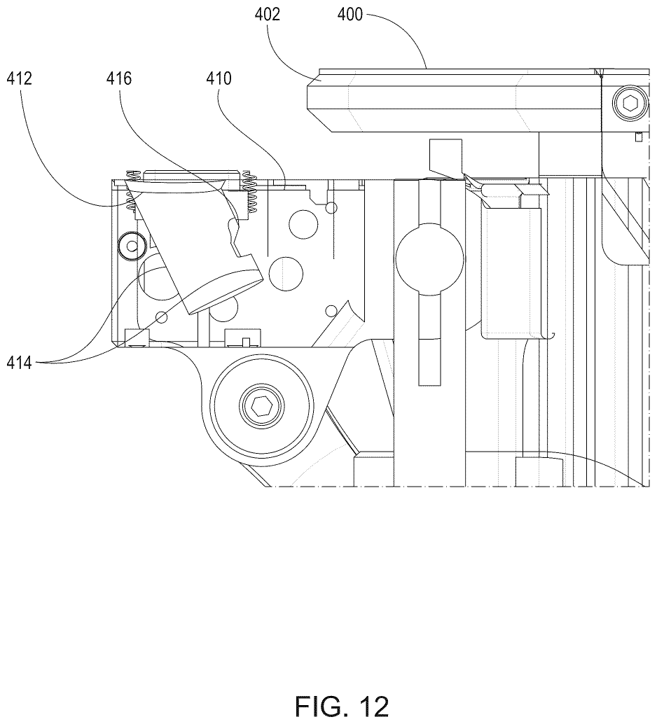

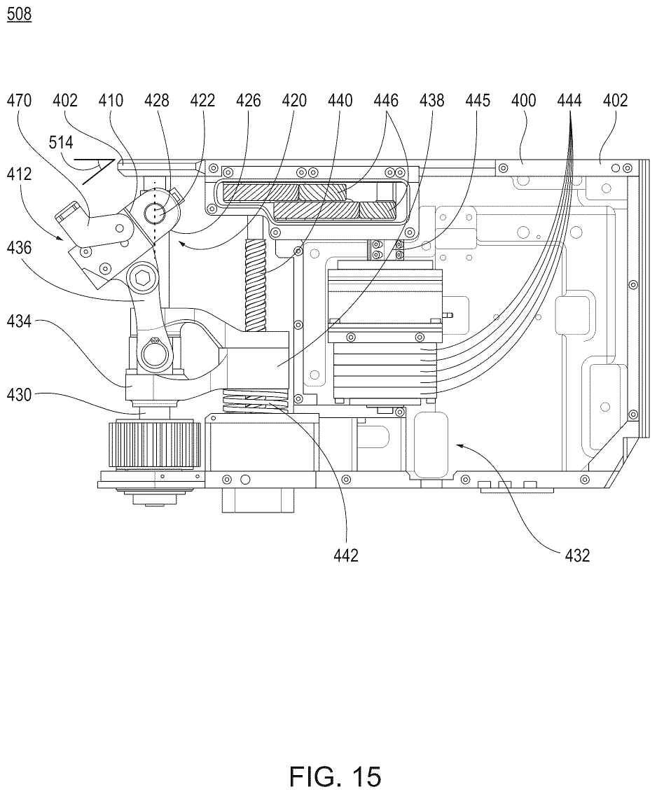

FIG. 15 shows lift assist mechanism 432 when spar 500 (FIG. 3) is in a pitched down configuration 508. With reference to FIG. 12, first spring 442 may provide lift assist for spar 500 to articulate spar 500 in a downward pitch direction, such as posterior direction 107 (FIG. 1). As shown in FIG. 12, first spring 442 may be compressed to allow spar mount 410 to articulate spar 500 in the downward pitch direction. Ball nut 438 may move in posterior direction 107 by rotation of screw 440 to compress first spring 442. Ball nut 438 may also be coupled to slider 434 such that slider 434 may translate along pivot post 430, also in posterior direction 107, thereby pulling linkage 436 in posterior direction 107 and causing spar mount 410 to be articulated downwardly. Spar mount 410 may be articulated such that a downward pitch angle 514 may form. In one embodiment, downward pitch angle 514 may be up to about 45 degrees, such as up to about 40 degrees. In other words, user 40 (FIG. 19) may pitch spar 500 downward in posterior direction 107 such that spar 500 is at downward pitch angle 514 relative to horizontal spar axis 510 (FIG. 3).

When spar is pitched downward to pitched down configuration 508 from straight spar configuration 506, first spring 442 may provide resistance as it compresses, reducing the weight felt by the user at the distal end of the spar 502. In the orientation of FIG. 15, spring 442 may be fully compressed and providing maximum force to spar 500 (FIG. 3) by way of spar mount 410, linkage 436, slider 434, and ball nut 438 all being coupled. Consequently, when user 40 (FIG. 19) begins raising spar 500, spring 442 may provide a “lift assist” to user 40 as spring 442 actuates in reverse. Spring 442 may provide a “lift assist” until spring 442 reaches the limit of its height, at which point it no longer provides a force against ball nut 438.

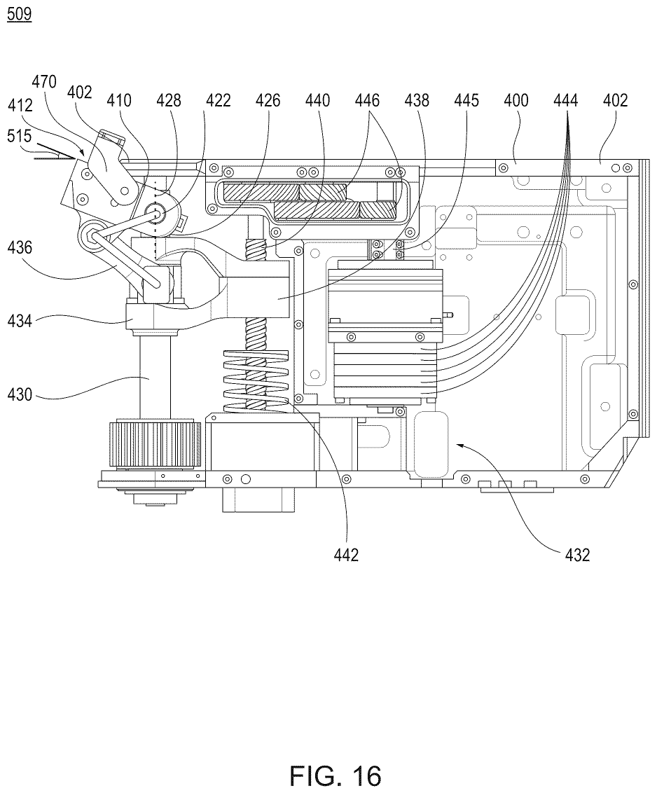

FIG. 16 shows lift assist mechanism 432 when spar 500 (FIG. 3) is in a pitched up configuration 509. In one embodiment, lift assist mechanism 432 may include gearbox 446 having one or more gears and one or more second springs 444. Each second spring 444 may include a mainspring coupled to a mainspring shaft 445 Mainspring shaft 445 may be coupled to gearbox 446, and gearbox 446 may be coupled to screw 440. In one embodiment, gearbox 446 may multiply the torque exerted by second springs 444 on mainspring shaft 445 incident on screw 440. The first spring 442, second springs 444 may reduce the weight felt by user 40 (FIG. 19) at distal end of the spar 502 when spar 500 (FIG. 1) is pitched downward to pitched down configuration 508. The mainsprings in second springs 444 are wound, and provide lift assist for spar 500 to articulate spar 500 in an upward pitch direction, such as anterior direction 105 (FIG. 1), as mainsprings in second springs 444 are unwound. One or more second springs 444 may include a plurality of springs stacked on top of one another, each second spring 444 coupled to mainsprings shaft 445, in order to provide torque for lift assist mechanism 432. In one embodiment, second springs 444 include one spring, one to three springs, and up to about six second springs 444 in a stacked configuration.

Similar to first spring 442, second springs 444 may be compressed or wound to allow spar mount 410 to articulate spar 500 in the downward pitch direction. Second springs 444 may store energy and may release stored energy when spar 500 (FIG. 3) is lifted, providing a “lift assist.” First spring 442 may be expanded to allow spar mount 410 to articulate spar 500 in the upward pitch direction. Ball nut 438 may move in anterior direction 105 by rotation of screw 440 to expand first spring 442. Ball nut 438 may also be coupled to slider 434 such that slider 434 may translate along pivot post 430, also in anterior direction 105, thereby pushing linkage 436 in anterior direction 105 and causing spar mount 410 to be articulated upwardly. Spar mount 410 may be articulated such that an upward pitch angle 515 may form. In one embodiment, upward pitch angle 515 may be up to about 30 degrees, such as up to about 21 degrees. In other words, user 40 (FIG. 19) may pitch spar 500 upward in anterior direction 105 such that spar 500 is at upward pitch angle 515 relative to horizontal spar axis 510 (FIG. 3). In one embodiment, lift assist mechanism 432 may include first spring 442. In another embodiment, lift assist mechanism 432 may include second springs 444 and gearbox 446. In another embodiment, lift assist mechanism 432 may include both first spring 442 and second springs 444, and gearbox 446.

Referring to FIGS. 3 and 15, as discussed, in one embodiment, user 40 (FIG. 19) may pitch spar 500 downward in posterior direction 107 such that spar 500 is at downward pitch angle 514 of up to about 45 degrees. Distal end 502 of spar 500 may reach a position proximate ground surface 14 (FIG. 1) when spar 500 is articulated in a downward pitch direction such that foot 36 (FIG. 20) of at least one leg 32 (FIG. 20) of patient 20 (FIG. 20) is proximate ground surface 14. Spar 500, and in some embodiments, distal end 502, may not include a distal traction mechanism, or a device for generating traction. Spar 500 not including a distal traction mechanism may shorten a length of spar 500 as additional structure distal to leg 32 of patient 20 is not needed. In other words, spar 500, and in some embodiments, distal end 502, may be generally coextensive with leg 32 of patient 20 and not extend substantially beyond leg 32. Spar 500 may not extend as far as a spar with a distal traction mechanism. In this way, spar 500 may be pitched further downward up to about 45 degrees.

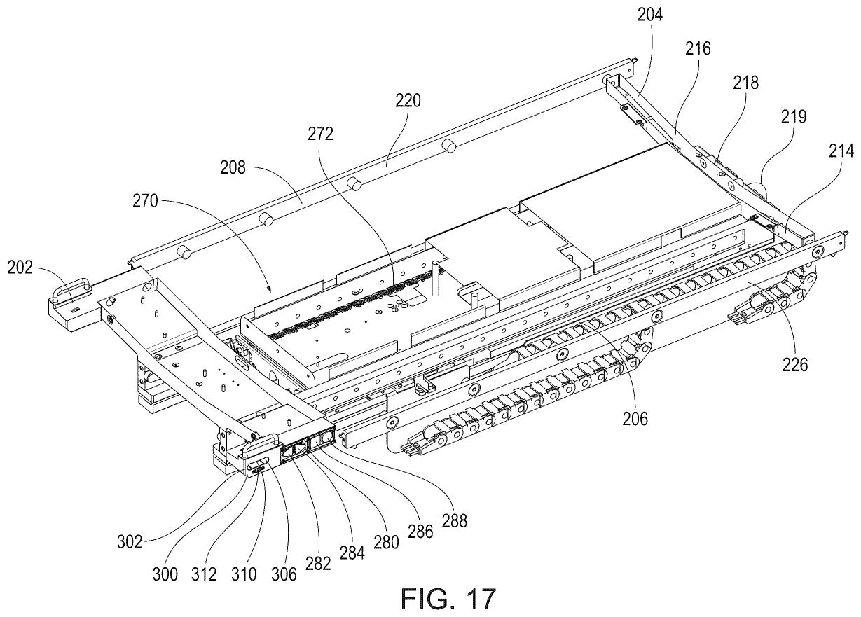

Surgery table 200 (FIG. 1) described herein may include a traction mechanism 270 as shown in FIG. 17 according to exemplary embodiments. However, instead of being at distal end 502 (FIG. 3) of spar 500, traction mechanism 270 may be along surgery table 200 (FIG. 1), such as along platform 220. Spar base 400 (FIG. 1) may be coupled to traction mechanism 270 such that a traction load is applied by traction mechanism 270 to at least one leg 32 (FIG. 20) of patient 20 (FIG. 20) as spar base 400 translates along surgery table 200. Instead of including a traction mechanism, distal end 502 of spar 500 may include handle 560 to allow user 40 to articulate spar 500, the handle 560 including one or more buttons, such as control 562 (FIG. 3), control 564 (FIG. 3), and control 565 (FIG. 3), to unlock spar 500 to allow spar 500 to be articulated. In addition, spar 500 may include foot support module 350 (FIG. 1) to support at least one foot 36 (FIG. 20) of at least one leg 32 of patient 20. Accordingly, distal end 502 may include one or more first spar controls to articulate spar 500 and foot support module 350. Thus, distal end 502 of spar 500 may extend minimally beyond leg 32 in inferior direction 103 (FIG. 1). Distal end 502 may include various features that do not include a traction mechanism. Spar 500 not including a traction mechanism may also reduce the weight of spar 500 to allow spar 500 to be more easily articulated by user 40 (FIG. 20).

FIG. 17 shows platform 220 without top portion 221 (FIG. 2) to view one or more lead screws 272, which may be coupled to spar base 400 (FIG. 1). Platform 220 may include lead screw 272 to translate spar base 400. Lead screw 272 may extend between distal end 202 and proximal end 204 of surgery table 200 (FIG. 1), such as platform 220. Accordingly, as spar base 400 translates a distance between distal end 202 and proximal end 204, a traction load may be applied or released. Lead screw 272 may apply traction as spar 500 (FIG. 3) is pushed outwardly from surgery table 200 in inferior direction 103 (FIG. 1). Lead screw 272 may reduce traction in the opposite direction. In other words, when patient is stationary on surgery table 200, and foot 36 of patient 20 (FIG. 20) is coupled to distal end 502 of spar 500, traction may be applied to leg 32 (FIG. 20) of patient 20 by moving the spar base 400 by lead screw 272, which may be coupled to spar 500, which may be coupled to foot support module 350, which may be coupled to foot 36 of patient 20. In one embodiment, electromechanical driver may include a profiled ball-bearing linear rail, and spar base 400 may translate along the profiled ball-bearing linear rail. In other embodiments, electromechanical driver may include a bracket (not shown) which may connect spar base 400 to a housing (not shown) containing a lead nut and a load cell. Electromechanical driver may include lead screw 272 passing through the lead nut such that force applied by lead screw 272 in applying traction is transmitted through the load cell to measure the traction force.

Surgery table 200 may also include a strain gauge (not shown) to measure the traction load and a display (not shown) to indicate a measurement of the traction load. The display may be located along surgery table 200 where user 40 (FIG. 19) may be located, such as along platform 220 or along spar 500, or on another user control. User 40 may then understand the traction load being applied to patient 20 (FIG. 20) and adjust accordingly.

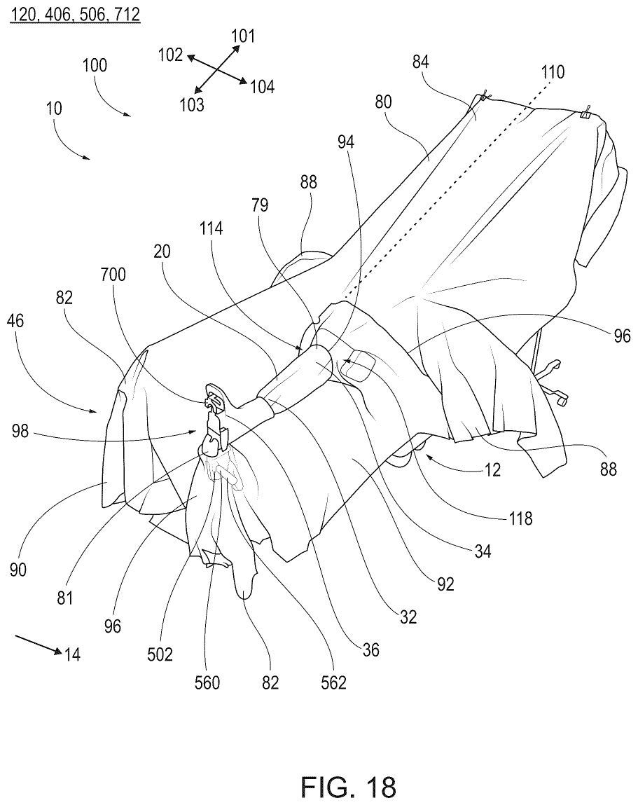

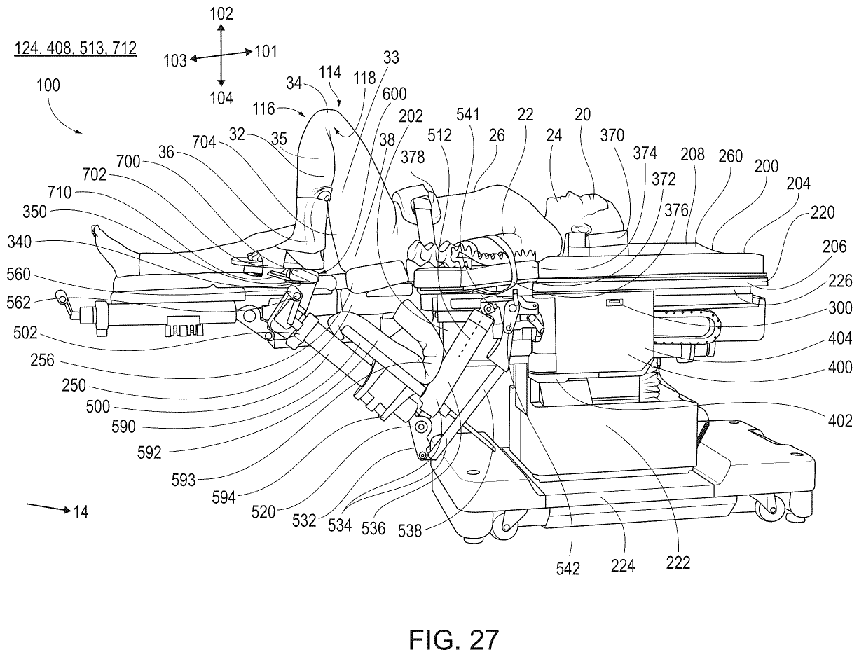

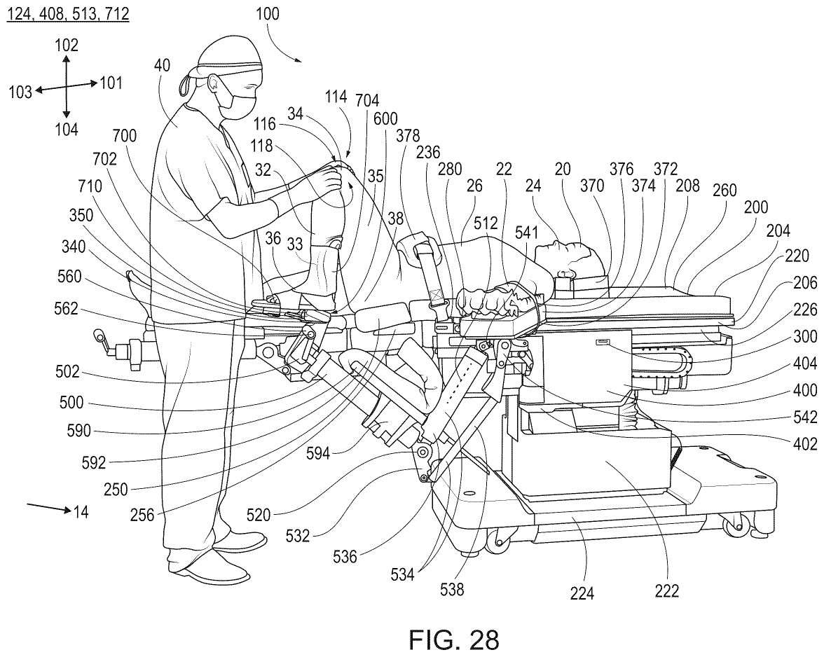

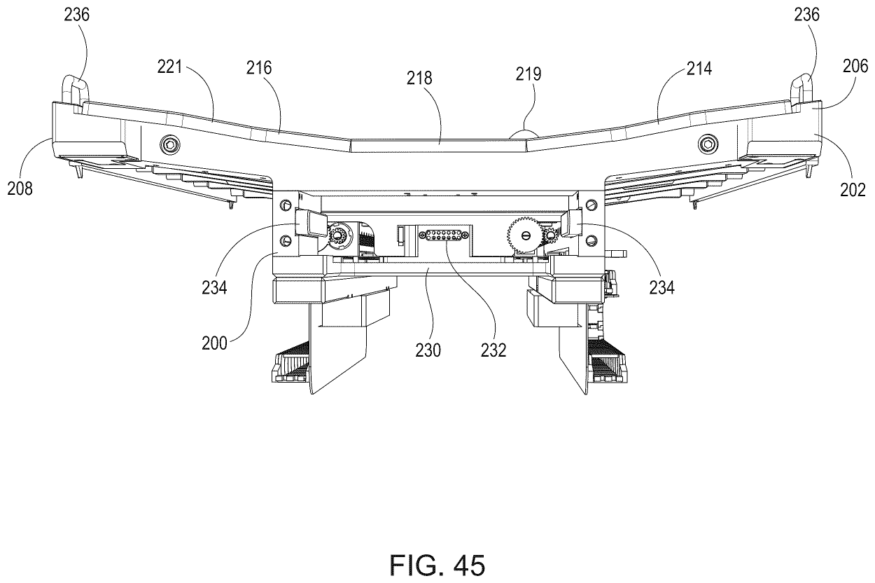

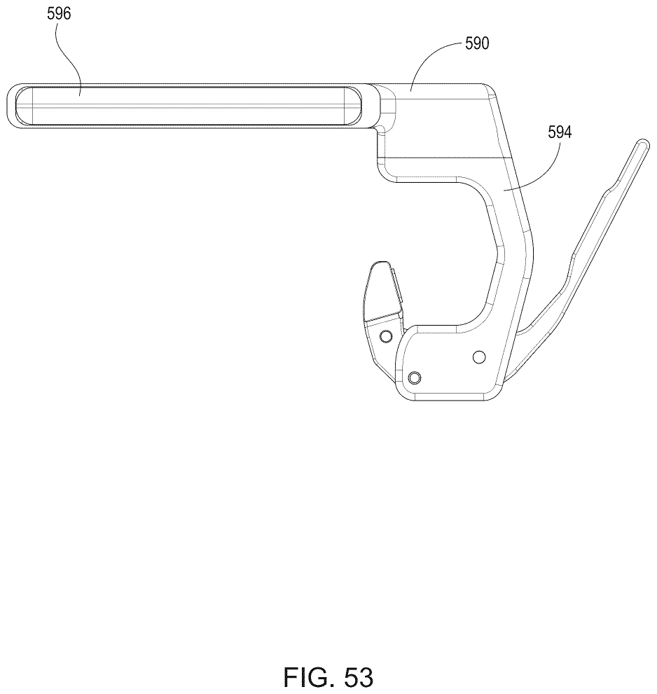

With reference to FIGS. 1-17, surgery table 200 may be convertible between a general-purpose surgery table and a specialty surgery table for orthopedic surgery, for example. In addition, surgery table 200 may be configured for knee surgery with one or more of the features described previously in addition to including features specific for knee surgery. Surgery table 200 configured for knee surgery will now be described with reference to FIGS. 18-66.

Positioning system 100 is shown in FIGS. 18-19 according to exemplary embodiments. In a first position 120 of positioning system 100, spar base 400 (FIG. 1) may be at a first position 406 at distal end 202 (FIG. 1) of surgery table 200 (FIG. 1), and spar 500 (FIG. 3) may be in straight spar configuration 506. As shown, a medial window 114 and a lateral window 118 may be formed proximate to spar 500 to provide direct medial and lateral access to knee 34. In other words, spars 500 may be separated to allow user 40, e.g., a practitioner such as a surgeon, medial access to a knee 34 of patient 20 from medial direction 102 in addition to lateral access from lateral direction 104. The practitioner may stand between spars 500 for direct lateral access to knee 34 from lateral direction 104. Spars 500 may be separated by articulating second joint 426 (FIG. 14), for example. In other words, one or more spars 500 may be moved laterally in a yaw motion.

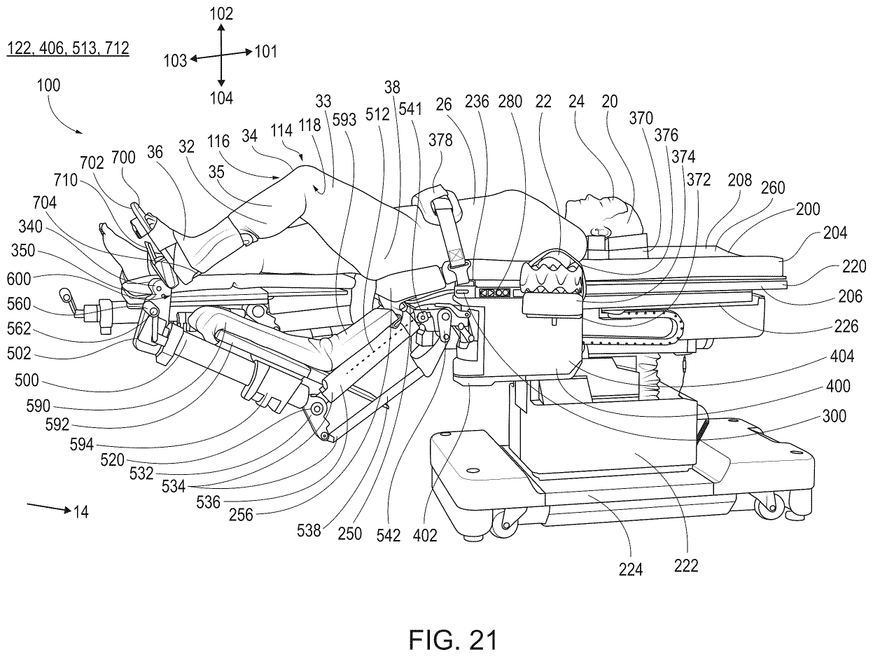

In FIGS. 20-25, positioning system 100 may be in a second position 122 according to exemplary embodiments. In second position 122, spar base 400 may be at distal end 202 of surgery table 200, and spar 500 may be in folded spar configuration 513. In FIGS. 20-25, spar 500 may be partially folded. In other words, spar 500 may be further folded, or flexed. As shown, spar 500 may flex such that distal end 502 folds toward proximal end 504, with proximal end 504 also folding and extending along a folded spar axis 512 rather than straight spar axis 510 (FIG. 3). As distal end 502 folds toward proximal end 504, an angle between distal end 502 and proximal end 504 may decrease from about 180 degrees in straight spar configuration 506 (FIGS. 18-19) up to about 90 degrees, and any range in between. As shown, medial window 114 and lateral window 118 may be formed proximate to spar 500 to provide direct medial and lateral access to knee 34. In addition, an inferior window 116 (FIGS. 21-29) may begin forming proximate to spar 500, and specifically distal end 502. In other words, user 40 may have medial access to knee 34 of patient 20 from medial direction 102, lateral access from lateral direction 104, and, as spar 500 is flexed, increasing inferior access to knee 34 of patient 20 from inferior direction 103. The practitioner may stand directly in front of knee 34 for inferior access to knee 34 without any intermediate structures, such as a traction device.

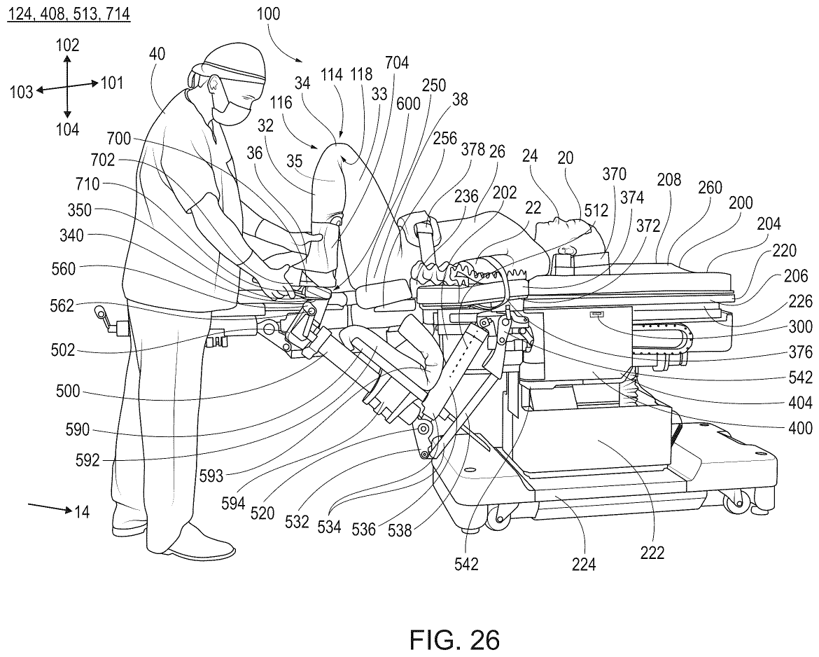

In FIGS. 26-29, positioning system 100 may be in a third position 124 according to exemplary embodiments. In third position 124, spar base 400 may be at a second position 408 between distal end 202 and proximal end 204 of surgery table 200, and spar 500 may be in folded spar configuration 513. Spar 500 may be flexed such that an upper leg 33 of leg 32 of patient 20 is proximate to surgery table 200. In this position, knee 34 may be fully flexed. In certain embodiments, the flexing may require spar base 400 to translate in superior direction 101 to accommodate spar 500 folding up and the angle between distal end 502 and proximal end 504 of spar 500 decreasing to about 90 degrees. As shown, medial window 114, lateral window 118, and inferior window 116 may be formed proximate to spar 500 to provide direct medial, lateral, and inferior access to knee 34. In other words, user 40 may have medial access to knee 34 of patient 20 from medial direction 102, lateral access from lateral direction 104, and inferior access to knee 34 of patient 20 from inferior direction 103. In other words, user 40 may stand proximate and inferior to the knee 34 without substantially leaning over a portion of surgery table 200 that was supporting knee 34 before knee 34 was bent.

Thus, spar 500 may fold to allow inferior access via inferior window 116. In other words, joint 520 may actuate to allow direct inferior access to knee 34 of patient 20. Knee 34 may be bent down such that knee 34 is anterior to foot 36 and spar 500 may be bent up such that joint 520 is posterior to distal end 502 and proximal end 504 of spar 500 when joint 520 is actuated to allow direct inferior access to knee 34. Inferior access may be supported by spar 500 being generally coextensive with leg 32 of patient 20 and not extending substantially beyond leg 32. As discussed above, spar 500 may be devoid of a distal traction mechanism. Instead, distal end 502 of spar 500 may include handle 560 to allow user 40 to articulate spar 500, the handle 560 including one or more buttons, such as control 562, control 564 (FIG. 3), and control 565 (FIG. 3), to unlock spar 500 to allow spar 500 to be articulated. In addition, spar 500 may include foot support module 350 to support at least one foot 36 of at least one leg 32 of patient 20.

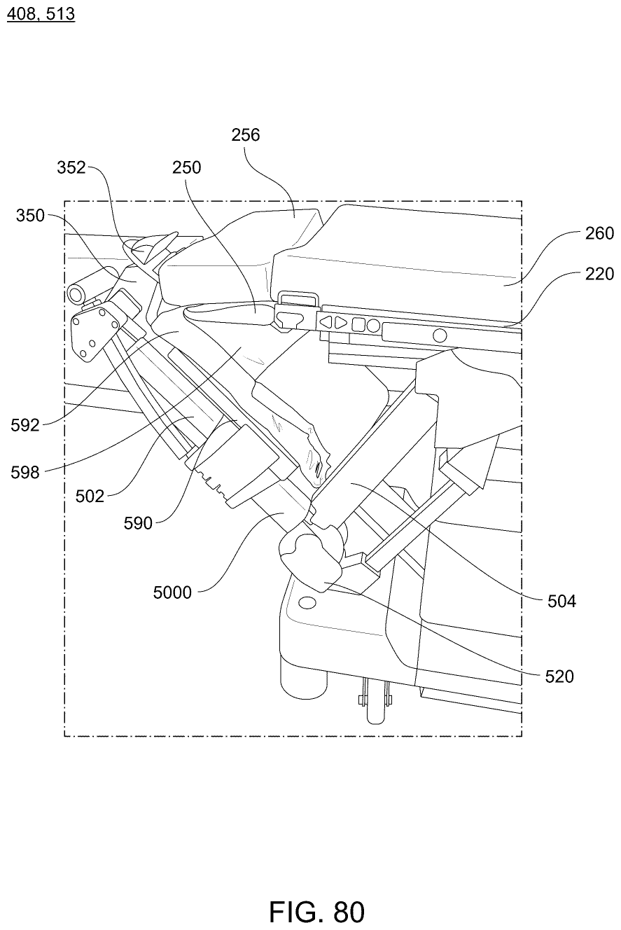

Spar base 400 in second position 408 as spar 5000 is in folded spar configuration 513 is also shown in FIG. 80 according to exemplary embodiments. Referring to FIGS. 26 and 80, the range of translation and flexation motion of spar base 400 and spar 5000, respectively, can position lower leg 35 of patient 20 proximate to gluteal region 38 of patient 20. As shown, a gripping mechanism 352, which will be discussed further below, can be proximate knee ledge board 250 as spar base 400 is translated and spar 5000 is flexed. Gripping mechanism 352 is coupled to foot 36 of patient 20 such that upper leg 33 of patient 20 is proximate gluteal region 38 of patient 20 disposed on knee ledge board 250. In this way, maximum translation of spar base 400 and flexation of spar 5000 can be achieved to facilitate inferior access of knee 34 of patient 20. As will be discussed spar 5000 may include any of the features of spar 500 and vice versa.

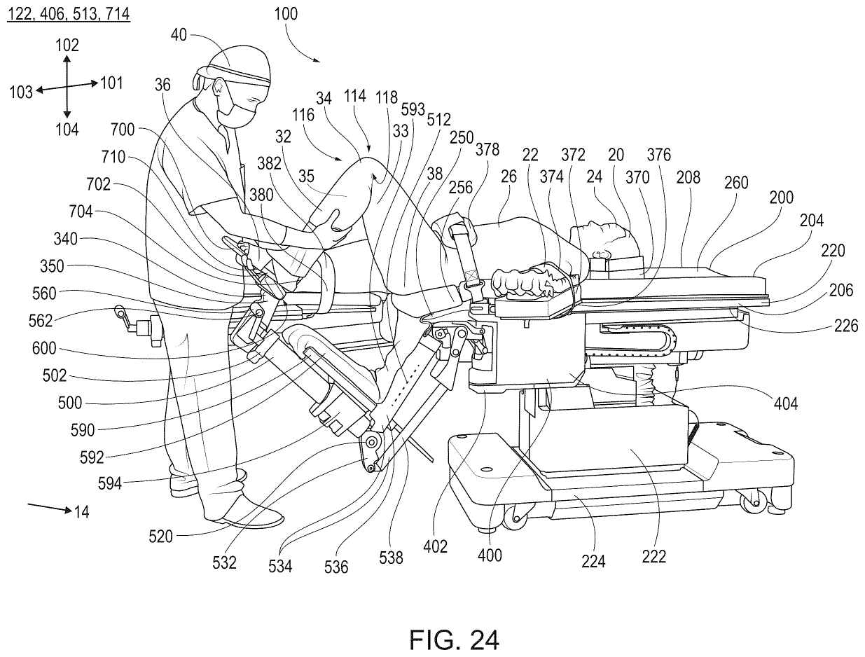

In certain embodiments, as spar 500 is both flexed and translated, as shown in FIGS. 26-29, positioning system 100 may compensate for any increase in height of knee 34. To maintain the height of knee 34, such as in comparison to knee 34 in FIG. 24 when knee 34 is not fully flexed, surgery table 200 may be lowered in posterior direction 104 to lower knee 34 such that knee 34 is at the same height as when knee 34 is not fully flexed, such as in FIG. 24. Column 222 may be shortened to lower spar 500, and therefore knee 34. Accordingly, as spar 500 is both flexed and translated, the height of surgery table 200 and corresponding spar 500 may be adjusted down. In other words, as knee 34 raises as it flexes, surgery table 200 may be lowered to keep knee 34 at a height between the waist and chest of user 40.

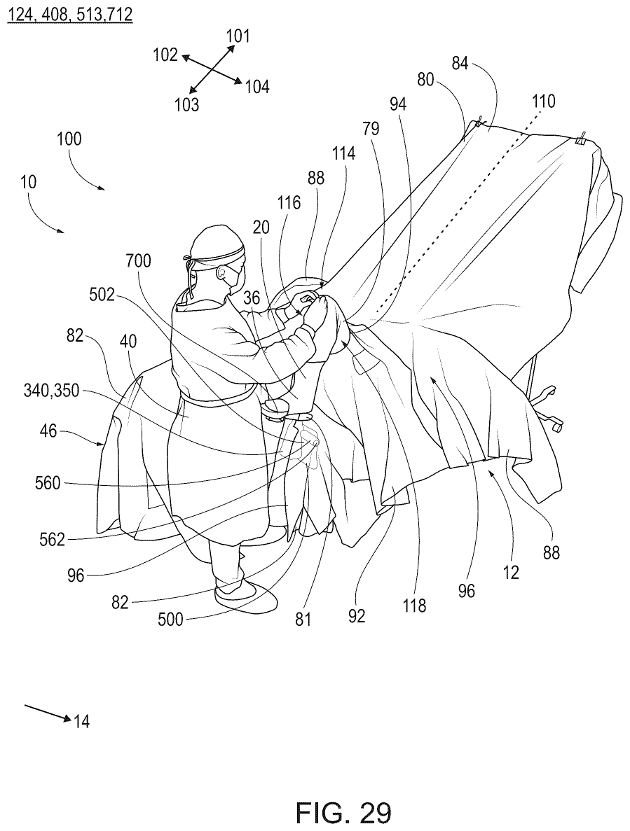

In a surgical procedure, it is important to maintain a sterile environment. Several tools may be used to create and maintain the sterile environment, such as one or more drapes 46. One or more drapes 46 may be seen in FIG. 29, for example. In an example, one or more drapes 46 may be part of positioning system 100. In another example, one or more drapes 46 may be a part of surgery table 200. In another example, one or more drapes 46 may function separately, such as with other positioning systems, surgery tables, or apparatuses thereof. In an example, one or more drapes 46 may include features that are functional separately and in combination.

With reference to FIG. 29, one or more drapes 46 may be used for positioning system 100 to separate a sterile space 10 and a working space 12. Sterile space 10 may include a surgical site, and working space 12 may include various equipment for facilitating a surgical procedure, such as surgery table 200 (FIG. 1) and spar 500. Working space 12 may also include non-operative equipment of positioning system 100. Working space 12 may also include portions of the patient that are not the subject of the surgical procedure, e.g., an arm of patient 20 if patient 20 is undergoing knee or hip surgery. Working space 12 may also generally include the area below a waist of user 40. Equipment and portions of patient 20 from sterile space 10 that are lowered below the waist of user 40 during the course of a surgical procedure may be considered non-sterile.

In certain embodiments, one or more drapes 46 may facilitate the surgical procedure as well. For example, as discussed, distal end 502 of spar 500 may include foot support module 350 to support at least one foot 36 of at least one leg 32 of patient 20. In addition, foot support 700 may support at least one foot 36 of at least one leg 32 of patient 20. Foot support module 350 may be coupled to foot support 700. However, foot support module 350 may be in working space 12. Foot support 700 may be in sterile space 10, as shown in FIG. 29. The coupling between foot support module 350 and foot support 700 will be described further below.

Distal end 502 of spar 500 and foot support module 350 may include various controls for articulating spar 500 and foot support 700, such as handle 560 having control 562 located in working space 12. Accordingly, user 40 who is positioned in sterile space 10 may require access to these controls through one or more drapes 46. Instead of lifting one or more drapes 46 to access these controls directly from working space 12 in which controls are disposed, which would breach the sterile barrier formed by one or more drapes 46, user 40 may access the controls from sterile space 10. As shown, one or more drapes 46 may include film, or transparent portions, that may allow user 40 visual access to controls such that user 40 may actuate controls from sterile space 10 through one or more drapes 46. For example, one or more drapes 46 may include one or more film portions disposed over handle 560 to allow user 40 to articulate spar 500. The film may be disposed over handle 560 such that user 40 may view handle 560 through one or more drapes 46.

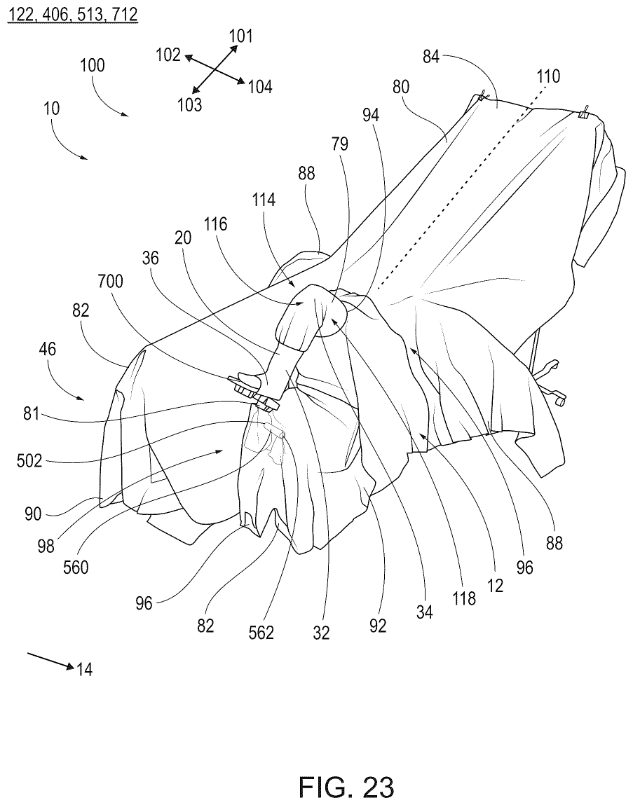

In other embodiments, one or more drapes 46 may include drapes to contact various parts of patient 20 and positioning system 100. For example, one or more drapes 46 may include one or more lower drapes to contact working space 12. In addition, one or more drapes 46 may include a top drape 80 to contact sterile space 10. As shown in FIGS. 23, 25, 29, 30, top drape 80 may include a distal end 82 that may extend at least to distal end 502 of spar 500. Top drape 80 may have an opposing proximal end 84. Between proximal end 84 and distal end 82, top drape 80 may have one or more arm portions 88. Arm portions 88 may cover one or more arms 22 (FIG. 20) of patient 20 as one or more arms 22 extend laterally from patient 20. Patient 20 may be positioned such that arms 22 extend laterally to allow for direct lateral access via lateral film 118 to knee 34 without arms 22 being in the way. Without arm portion 88, arms 22 would be proximate leg 32 and therefore intermediate to leg 32 and user 40.

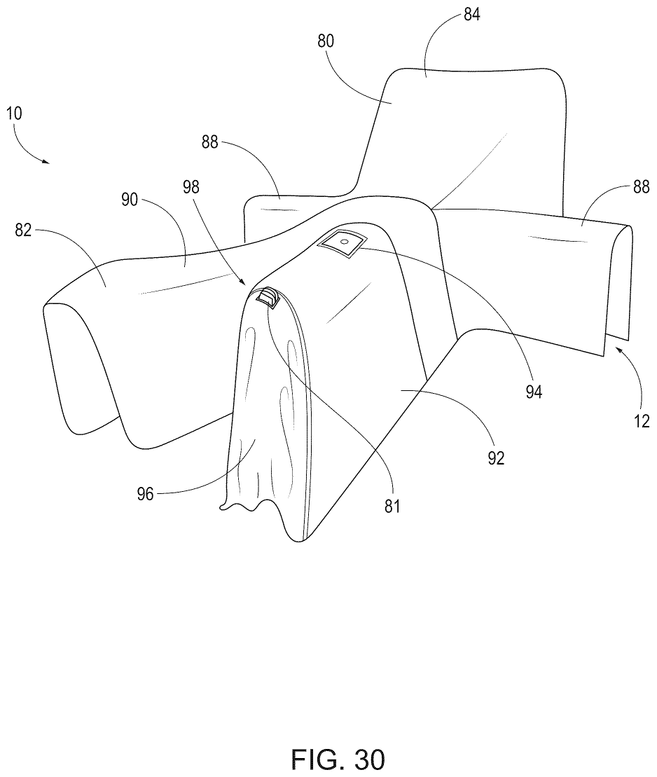

In embodiments, top drape 80 may have a leg opening 94 to receive leg 32 of patient 20, as top drape 80 is disposed between patient 20 and sterile space 10. Leg 32 that is received by leg opening 94 by top drape 80 may be the operable leg, which may be the left leg as shown in FIGS. 29, 30.

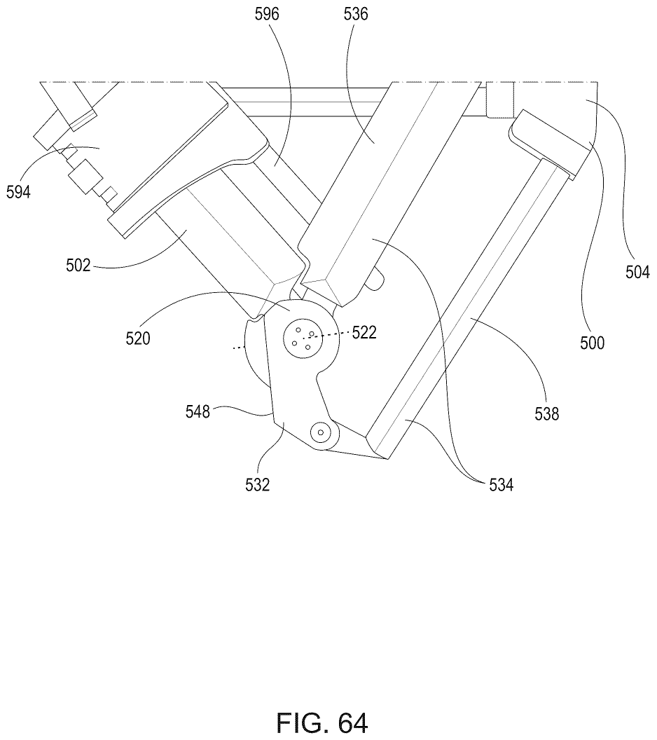

Top drape 80 may have one or more film portions 96. Film portions 96 may be transparent to form one or more windows through top drape 80, while maintaining a sterile barrier. Top drape 80 may be shown with reference to FIGS. 29-30 according to exemplary embodiments. As shown, film portion 96 may be between distal end 82 and proximal end 84 of top drape 80, such as proximate to arm portion 88. In this way, film portion 96 may be disposed over a portion of surgery table 200 (FIG. 1), such as a portion of platform 220 (FIG. 1), such as over one or more traction controls 280 (FIG. 62) disposed on platform 220, which will be described further below. In this way, user 40 may view traction controls 280 through one or more drapes 46. In another example, a separate film portion may be disposed over at least a portion of joint 552 (FIG. 3) of spar 500. Center joint 552 may include one or more controls for center joint 552, such as a manual lock 548 (FIG. 64), which will be described further below. In this way, user 40 may view manual lock 548 through one or more drapes 46. Film portion 96 may be disposed over distal end 502 of spar 500. In an example, film portion 96 may be disposed over handle 560 to allow user 40 to articulate spar 500. Film portion 96 may be disposed over handle 560 such that user 40 may view handle 560 and its controls through one or more drapes 46. In another example, film portion 96 may be disposed over foot support module 350. Film portion 96 may be disposed over foot support module 350 such that user 40 may view foot support module 350 and its controls through one or more drapes 46. Top drape 80 may be deployed last in a process to deploy one or more drapes 46.

As shown in FIGS. 29-30, top drape 80 may include one or more hard points 81. Hard point 81 may be made of rubber or polymer material. Hard point 81 may be integral with top drape 80. Hard point 81 may be disposed along top drape 80 to allow coupling between components of positioning system 100 in sterile space 10 with components of positioning system 100 in working space 12 without breaking the sterile barrier formed by one or more drapes 46. Accordingly, hard point 81 may be a sterile interface that may allow components in sterile space 10 to remain sterile when coupling to components in working space 12. Top drape 80 may be substantially similar to or identical to drapes described in U.S. Provisional Application No. 63/712,500 filed Oct. 27, 2024 titled “Drape for Use in Surgical Procedures” and U.S. patent application Ser. No. 19/370,426 filed Oct. 27, 2025 titled “Drape for Use in Surgical Procedures,” which are hereby incorporated by reference in their entireties. Hard point 81 may be substantially similar or identical to hard points described in U.S. Provisional Application No. 63/712,500.

For example, positioning system 100 may include table modules 340, such as foot support module 350. Table module 340 may couple to surgical equipment across one or more drapes 46, table module 340 being in working space 12 and the surgical equipment being in sterile space 10. For example, foot support module 350 may couple to foot support 700 across one or more drapes 46. Accordingly, foot support 700 may be in sterile space 10 and foot support module 350 may be in working space 12. Foot support 700 may couple to foot support module 350 via hard point 81. Foot support 700 in sterile space 10 may couple to foot support module 350 in working space 12 via hard point 81 such that hard point 81 is a sterile interface that may selectively and repeatedly couple components across a sterile barrier without breaching the sterile barrier.

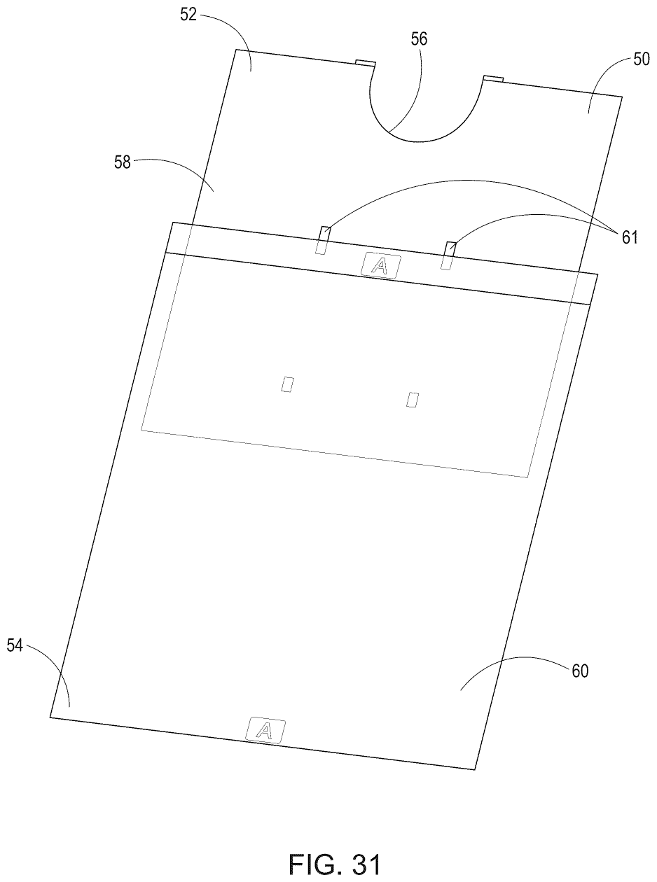

One or more lower drapes of one or more drapes 46 may include a first lower drape 50 shown in FIG. 31 according to exemplary embodiments. With reference to FIGS. 29-30, first lower drape 50 may contact working space 12. First lower drape 50 may be deployed first in a process to deploy one or more drapes 46. First lower drape 50 may include a distal end 52 that may extend over at least a portion of leg 32 of patient 20. First lower drape 50 may have an opposing proximal end 54. First lower drape 50 may have a leg cutout 56 to wrap around leg 32 of patient 20, such as an underside of leg 32 as first lower drape 50 is disposed between patient 20 and working space 12. Leg 32 that is wrapped by first lower drape 50 may be the operable leg, the left leg shown in FIG. 29. Leg cutout 56 may have a U-shape. Leg cutout 56 may be at proximal end 54 of first lower drape 50. First lower drape 50 may be placed on spar 500, and then leg 32 may be placed on top of first lower drape 50. Terminal edges of leg opening 56 may then be raised around the underside of operable leg 32, and secured to the top, or upper thigh area, of operable leg 32 such that leg opening 56 encircles operable leg 32 at the top. Leg opening 56 may be secured to leg by a fastener, such as adhesive, tape, a button, or a hook and loop fastener, for example. First lower drape 50 may have fabric 58 at proximal end 52. In addition, first lower drape 50 may have a film portion 60. Film portion 60 may be transparent to form a window through first lower drape 50. Film portion 60 may be at distal end 52 of first lower drape 50.

Film portion 60 may be disposed over distal end 502 of spar 500 during the draping process. Once top drape 80 is deployed and covering distal end 502 of spar 500, film portion 60 may be removed from first lower drape 50. As such, in certain embodiments, film portion 60 is removable, or sacrificed, during the draping process. As shown in FIG. 31, first lower drape 50 may include one or more fasteners, such as adhesive, to couple film portion 60 to fabric 58 of first lower drape 50. Film portion 60 may be removed such that only top drape 80 is disposed between sterile space 10 and working space 12 at distal end 502 of spar 500, as show in FIG. 29. Film portion 60 may be removed so that hard point 81 of top drape 80 may couple to foot support module 350.

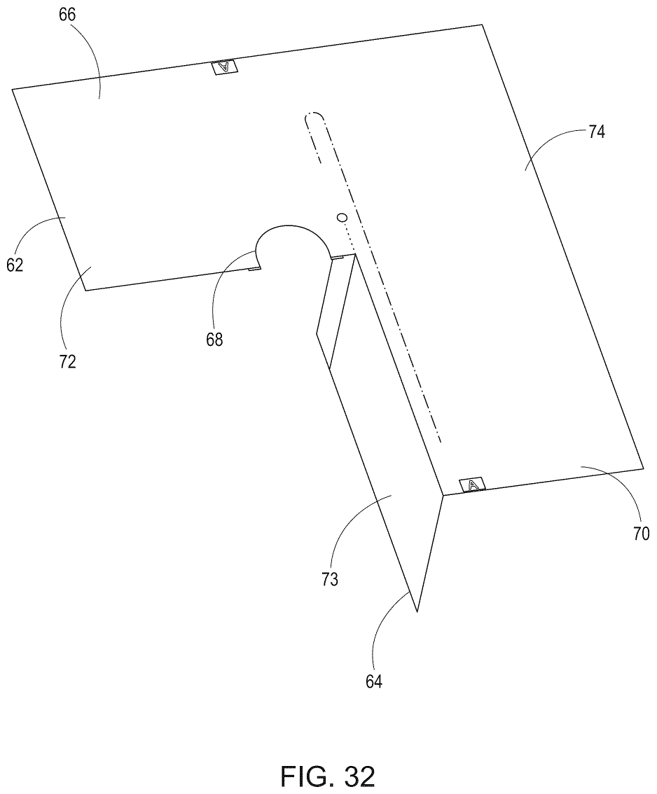

One or more lower drapes of one or more drapes 46 may include a second lower drape 62 shown in FIG. 32 according to exemplary embodiments. Second lower drape 62 shown may be for a right leg operation, rather than a left leg operation shown herein. Second lower drape 62 functions the same whether the right leg or the left leg is operable, with its components being flipped to be compatible for each operation. Second lower drape 62 may contact sterile space 10 and working space 12, and may form the boundary therebetween.

With reference to FIGS. 29-32, at least a portion of second lower drape 62 may be disposed between first lower drape 50 and top drape 80. Second lower drape 62 may include a distal end 64 that may extend at least to distal end 502 of spar 500. Second lower drape 62 may have an opposing proximal end 66. Second lower drape 62 may have a leg opening 68 to wrap around operable leg 32 of patient 20. Leg opening 68 may have a U-shape. Leg opening 68 may be disposed between proximal end 66 and distal end 64 of second lower drape 62. Leg 32 that is covered by second lower drape 62 may be the non-operable leg. Second lower drape 62 may include fabric 70 from proximal end 66 to distal end 64.

With reference to FIGS. 29 and 32, second lower drape 62 may include two leg portions. Second lower drape 62 may include an operable leg portion 72 and a non-operable leg portion 74. Operable leg portion 72 may be coupled to non-operable leg portion 74. Non-operable leg portion 74 may be uncoupled from a leg cutout 68.

In a draping process, second lower drape 62 may be deployed after first lower drape 50 (FIG. 31) is deployed. Second lower drape 62 may be disposed over most of non-operable leg 32 of patient 20 such that second lower drape 62 is at least partially disposed between patient 20 and sterile space 10. As shown, non-operable leg portion 74 may be disposed over most of non-operable leg 32. Second lower drape 62 may be disposed over at least a portion, or all, of non-operable leg 32, such as the upper thigh area. Operable leg portion 72 may be disposed over a portion of operable leg 32 and may also fall between non-operable leg 32 an operable leg 32. Second lower drape 62 may have leg opening 68 to wrap around operable leg 32 of patient 20, such as an underside of non-operable leg 32 as second lower drape 62 is disposed between patient 20 and sterile space 10. Leg 32 that is wrapped by second lower drape 62 may be the operable leg. Leg opening 68 may have a U-shape. Leg opening 68 may be disposed over leg opening 56 (FIG. 31) of first lower drape 50.

Terminal edges of leg opening 56 may then be raised around the underside of operable leg 32 and secured to the top, or upper thigh area, of operable leg 32 such that leg opening 68 encircles the operable leg 32 at the top. Leg opening 68 may be secured to operable leg 32 by a fastener, such as adhesive, a button, or a hook and loop fastener, for example. Operable leg portion 72 may separate from leg opening 68, as shown in FIG. 32. In other words, from leg opening 68 to distal end 64, operable leg portion 72 may drop between leg opening 68 and non-operable leg portion 74. In this way, operable leg portion 72 may include a wall portion 73, shown in FIG. 32, that drops between operable leg 32 and non-operable leg 32 to form a sterile barrier therebetween. In other words, non-operable leg portion 74 may cover at least a portion of non-operable leg 32 and wall portion 73 may be disposed between operable leg 32 (FIG. 29) and non-operable leg 32. Referring to FIG. 29, separating non-operable leg portion 72 and operable leg portion 74 may allow user 40 to be disposed between legs 32 without breaching the sterile barrier as wall portion 73 may fall between legs 32 to allow user 40 to walk between legs 32 with spars 500 being separated.

In one embodiment, one or more drapes 46 may include a leg sleeve 79 as shown in FIG. 29. In one embodiment, leg sleeve 79 may include a sleeve of drape material including an open end and a closed end. Leg 32 of patient 20 may be inserted into the open end, with foot 36 located in the closed end, and the open end secured around leg 32 of patient 20, such as the upper thigh area, of leg 32. Leg sleeve 79 may be fastened to leg 32 such as with adhesive, a button, a hook and loop fastener, or an elastic band.

In a draping method for knee surgery, one or more drapes 46 may be deployed to create and maintain the sterile barrier, with reference to FIGS. 29-32. One or more lower drapes 46 may first be placed over at least a portion of patient 20 and positioning system 100. First lower drape 50 may be deployed first among one or more drapes 46, for example. First lower drape 50 may be disposed over at least a portion of spar 500 supporting operable leg 32 and table module 340, such as foot support module 350, that may be coupled to spar 500. Film portion 60 of first lower drape 50 may be disposed over distal end 502 of spar 500 during the draping process. Once top drape 80 is deployed and covering distal end 502 of spar 500, film portion 60 may be removed from first lower drape 50. First lower drape 50 may have leg opening 56 to wrap around operable leg 32 of patient 20, such as an underside of operable leg 32 as first lower drape 50 is disposed between patient 20 and working space 12. Leg cutout 56 may be secured to the top, or upper thigh area, of operable leg 32 such that leg opening 56 encircles operable leg 32 at the top. Film portion 60 may be at distal end 52 of first lower drape 50.

Second lower drape 62 may then be deployed. Second lower drape 62 may be disposed over at least a portion of operable leg 32, such as the upper thigh area, and over most of non-operable leg 32 of patient 20. Second lower drape 62 may have leg opening 68 to wrap around operable leg 32 of patient 20, such as an underside of operable leg 32 as second lower drape 62 is disposed between patient 20 and sterile space 10. Leg opening 68 may be secured to the top, or upper thigh area, of operable leg 32 such that leg opening 68 encircles operable leg 32 at the top. With leg opening 68 secured to upper leg 32 of operable leg 32, wall portion 73, may drop between operable leg 32 and non-operable leg 32 to form a sterile barrier therebetween.

Next, leg sleeve 79 may be applied to operative leg 32 and secured. Top drape 80 may then be deployed. Top drape 90 may be disposed over at least a portion of patient 20 and positioning system 100, such as over at least a portion of operable leg 32, such as the upper thigh area, and over most of non-operable leg 32 of patient 20. Top drape 80 may have leg opening 94 to wrap around operable leg 32 of patient 20, such as an underside of operable leg 32 as top drape 80 is disposed between patient 20 and sterile space 10. Leg opening 94 may be secured to the top, or upper thigh area, of operable leg 32 such that leg opening 94 encircles operable leg 32 at the top.

Toward proximal end 204 (FIG. 1) of surgery table 200 (FIG. 1), top drape 80 may be disposed over proximal end 204. Top drape 80 may include arm portion 88 to cover one or more arms 22 (FIG. 20) of patient 20 as one or more arms 22 extend laterally from patient 20. Toward distal end 502 of spar 500, film portion 96 of top drape 80 may be disposed over distal end 502 of spar 500 during the draping process. Film portion 96 may be disposed over handle 560 such that user 40 may view handle 560 and its controls through one or more drapes 46. In another example, film portion 96 may be disposed over foot support module 350. Film portion 96 may be disposed over foot support module 350 such that user 40 may view foot support module 350 and its controls through one or more drapes 46.

Once top drape 80 is deployed and covering distal end 502 of spar 500, film portion 60 may be removed from first lower drape 50. Top drape 80 may include hard point 81 that may couple to foot support module 350 in working space 12 and foot support 700 in sterile space 10. Therefore, foot support 700 may couple to foot support module 350 via hard point 81 across the sterile barrier formed by one or more drapes 46.

In a draping method for knee surgery, one or more drapes 46 may be deployed to create and maintain the sterile barrier, with reference to FIGS. 29-32, first lower drape 50 may be deployed first among one or more drapes 46, for example. First lower drape 50 may be disposed over at least a portion of spar 500 supporting operable leg 32 and table module 340, such as foot support module 350, that may be coupled to spar 500. Second lower drape 62 may then be deployed. Second lower drape 62 may be disposed over at least a portion of operable leg 32, such as the upper thigh area, and over most of non-operable leg 32 of patient 20. Second lower drape 62 may include wall portion 73 that may drop between operable leg 32 and non-operable leg 32 to form a sterile barrier therebetween. Next, leg sleeve 79 may be applied to operative leg 32 and secured. Top drape 80 may then be deployed. Top drape 90 may be disposed over at least a portion of patient 20 and positioning system 100, such as over at least a portion of operable leg 32, such as the upper thigh area, and over most of non-operable leg 32 of patient 20. Toward distal end 502 of spar 500, film portion 96 of top drape 80 may be may be disposed over distal end 502 of spar 500 during the draping process such that user 40 may view one or more controls through one or more drapes 46. Once top drape 80 is deployed and covering distal end 502 of spar 500, film portion 60 may be removed from first lower drape 50. Top drape 80 may include hard point 81 that may couple to foot support module 350 in working space 12 and foot support 700 in sterile space 10. Therefore, foot support 700 may couple to foot support module 350 via hard point 81 across the sterile barrier formed by one or more drapes 46.

With reference to FIGS. 18-30, as discussed, a medial window 114 and a lateral window 118 may be formed proximate to spar 500 to provide direct medial and lateral access to knee 34. In other words, spars 500 may be separated to allow user 40, e.g., a practitioner such as a surgeon, medial access to a knee 34 of patient 20 from medial direction 102 in addition to lateral access from lateral direction 104. Spars 500 may be separated by articulating second joint 426 (FIG. 14), for example. In other words, spars 500 may be moved laterally in a yaw motion. Non-operable and operable legs 32 may be draped with second lower drape 62 forming a sterile barrier therebetween. Accordingly, draping over non-operable and operable legs 32 may form a sterile zone 98 between non-operable and operable legs 32. User 40 may stand in between spars 500 and non-operable and operable legs 32 within sterile zone 98. In this way, user 40 may have direct medial and lateral access to both legs 32 while both legs 32 are supported on spars 500.

With reference to FIGS. 18-29, surgery table 200 may include foot support 700. Distal end 502 of spar 500 may include foot support module 350 to support at least one foot 36 of at least one leg 32 of patient 20. Foot support 700 may couple to foot support module 350 to couple to spar 500. Foot support module 350 may extend outwardly from spar 500 in medial direction 102 such that such that spar 500 is laterally offset from at least a portion of leg 32 of patient 20. Foot support module 350 may support foot 36 in working space 12 while foot support 70 may support foot 36 in sterile space 10.

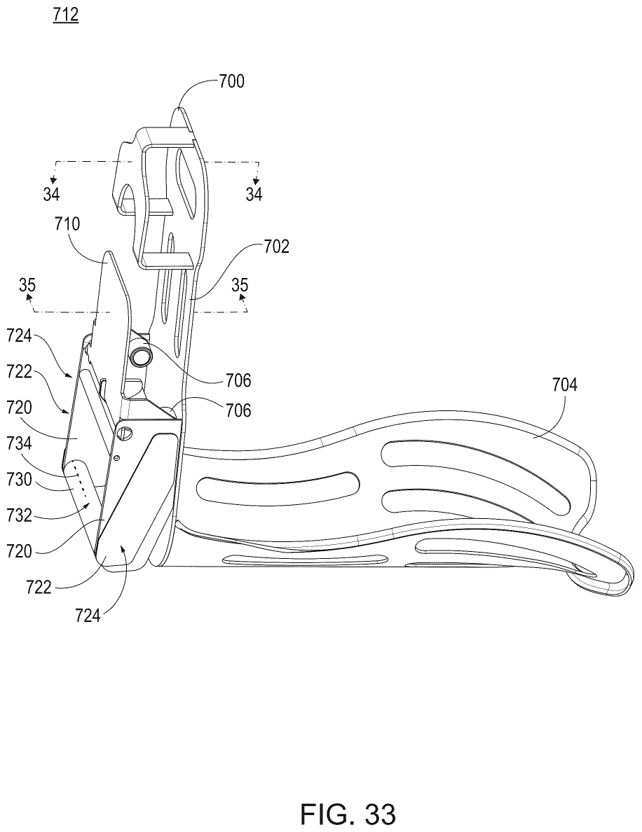

Foot support 700 is shown in FIGS. 33-35 according to exemplary embodiments. In an example, foot support 700 may be part of positioning system 100. In another example, foot support 700 may be a part of surgery table 200. In another example, foot support 700 may function separately, such as with other positioning systems, surgery tables, or apparatuses thereof. In an example, foot support 700 may include features that are functional separately and in combination.

In one embodiment, foot support 700 may include a foot portion 702, which may be proximate a foot 36 (FIG. 20) of patient 20 (FIG. 20). Foot support 700 may include a calf portion 704, which may be proximate at least a portion of leg 32 (FIG. 20), e.g. a calf, of patient 20. Foot support 700 may be generally rigid and may receive one or more disposable pads to support patient 20. To maintain sterility between patient 20, foot support 700 itself may be sterilizable, and may receive one or more disposable liners over pads.

Foot support 700 may include stainless steel for sterilizability. Foot support 700 may therefore be heat, corrosion, and chemical resistant. Features of foot support 700 may advance sterilizability, including non-porous surfaces, curved edges rather than tight internal corners, lack of small orifices, simple geometries, and open hinges rather than enclosed bearings. Reprocessing can include primarily steam sterilization following washer-disinfector cycles at high heat, with manual brushing as needed to remove biological matter.

Foot support 700 may also include a user interface paddle 710 having an extended position 712. User interface paddle 710 may be pivoted outward of extended position 712, as shown in FIG. 19. In this way, a wrapping may secure around foot 36 and foot portion 702 of foot support 700 to secure foot 36 to foot support 700. Once the wrapping is complete, user interface paddle 710 may be rotated back into extended position 712.

In embodiments, user interface paddle 710 may include a hinge end and a paddle end. Hinge end may include a hinge coupling user interface paddle 710 to the underside of foot support 700. Hinge may include an open hinge comprising a reed inside of an orifice. Open hinge may be configured to allow foot support 700 to be sterilizable. Paddle end may be configured for the hand of a user to engage and depress paddle end. User interface paddle 710 may include one or more magnets 740, and in one embodiment, two magnets 740. User interface paddle 710 may include a magnet mount portion generally opposite hinge, and inside of a side wall of foot support 700. When user interface paddle 710 is rotated from extended position to actuated position, user interface paddle 710 rotates about the hinge, causing corresponding rotation of magnet mount portion and magnets 740. Rotation of user interface paddle 710 may cause magnets to rotate from a position not adjacent to mu metal pathway, to a mu metal pathway activation position, where magnets 740 are adjacent to the mu metal pathway.

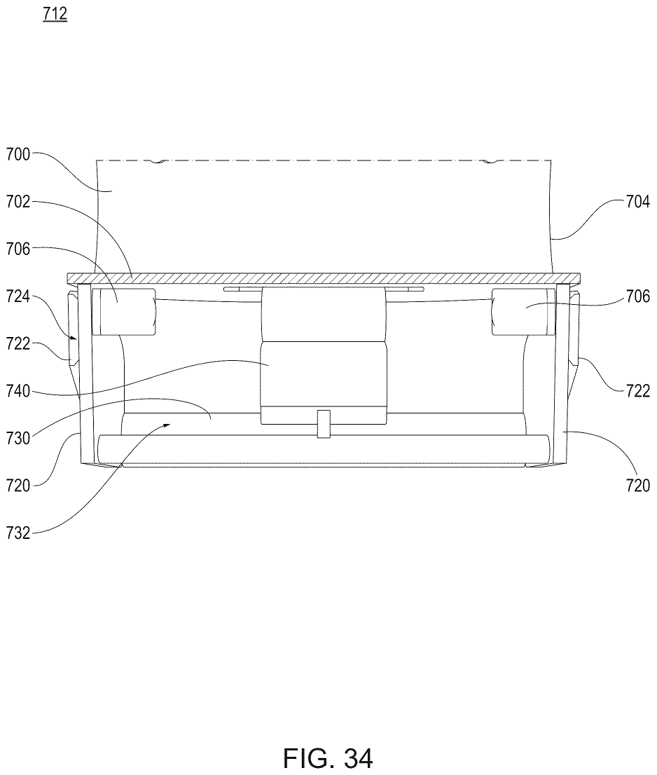

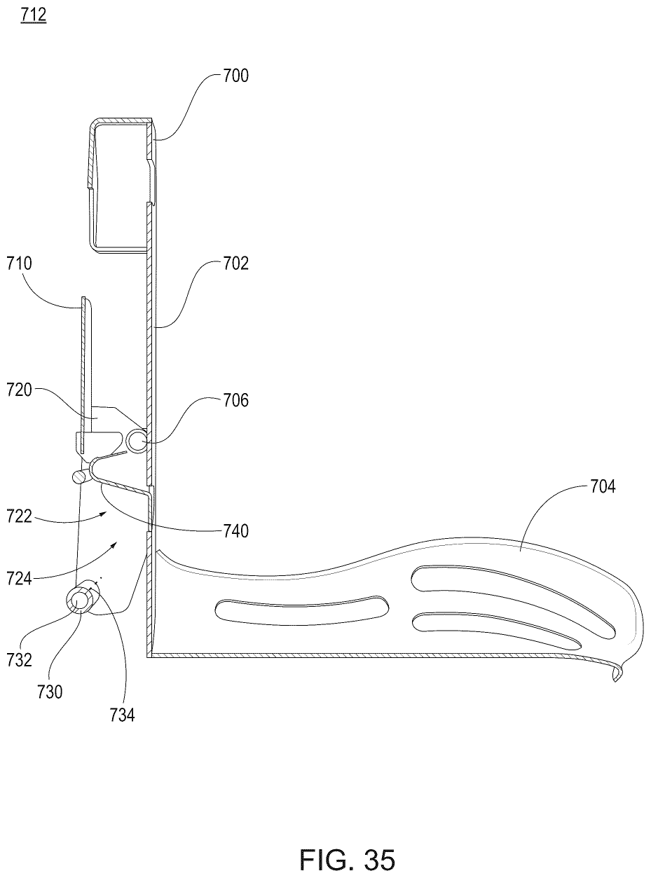

User interface paddle 710 may be actuated by being rotated inward. Foot support 700 may include one or more springs 740 to bias user interface paddle 710 to extended position 712. Accordingly, one or more magnets 706 normally do not generate the magnetic field in the rest of foot support 700 as user interface paddle 710 is biased to extended position 712 in which one or more magnets 706 do not generate the magnetic field in other parts of foot support 700. To generate the magnetic field in other portions of foot support 700, user interface paddle 710 is actuated to an actuated position 714, shown in FIGS. 24-27, for example. In actuated position 714, one or more magnets 706 may rotate as user interface paddle 710 is rotated inward. As shown with reference to FIGS. 34-35, user interface paddle 710 is in extended position 712. When user interface paddle 710 is pivoted inward to actuated position 714, magnets 706 may rotate into position adjacent mu metal conduit 722.

When user interface paddle 710 is actuated, one or more magnets 706 rotate to be adjacent to mu metal conduit 722. Mu metal may be metal that is easily magnetized and demagnetized. In embodiments, mu metal not permanently magnetize. Accordingly, a magnetic interface may be formed between one or more magnets 706 and mu metal conduit 722 when one or more magnets 706 is adjacent mu metal conduit 722 in sidewall 720. However, it is not necessary for the one or more magnets 706 to touch the adjacent sidewall 720 or mu metal conduit 722, in certain embodiments, it is sufficient for the one or more magnets 706 to be near to, and separated by a gap from, the mu metal conduct 722. Mu metal conduit 722 may extend along, and be continuous along, sidewall 720 to a magnetic terminus, such as at rod 730, to form a magnetic highway. In other words, mu metal conduit 722 may be a conduit for a magnetic flux. In this way, the magnetic field may be extended from magnets 706 beyond sidewall 720, through the mu metal conduit 722 to a magnetic terminus. In one embodiment, the magnetic terminus includes rod 730.

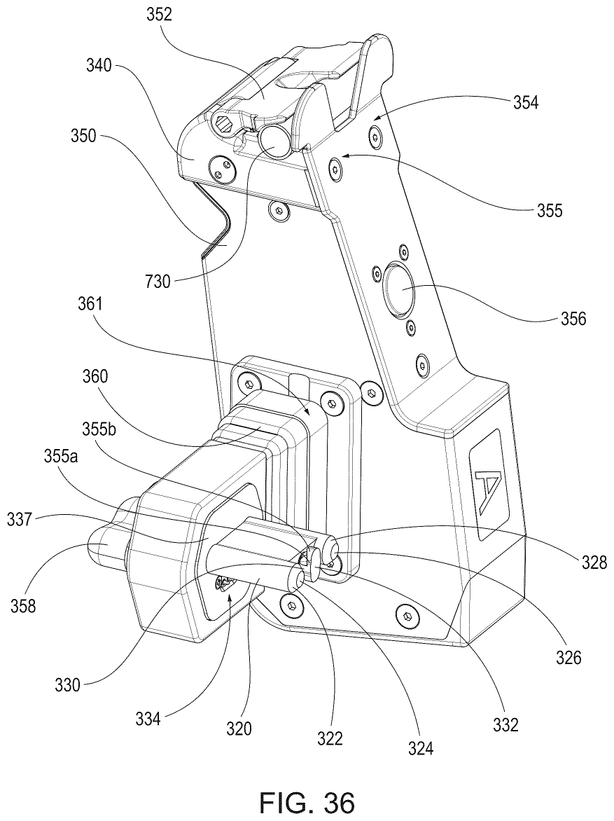

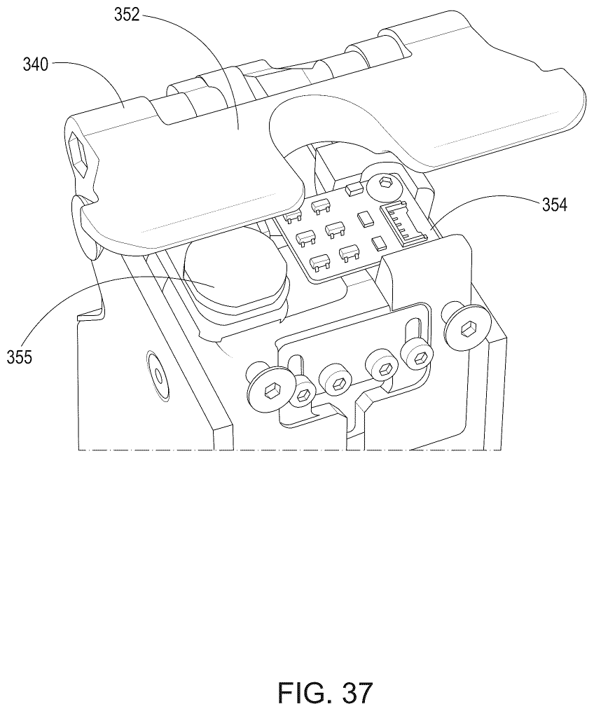

Foot support 700 may also include a rod 730. With reference to FIG. 31, rod 730 may include mu metal 732. Mu metal 732 may be enclosed in rod 730 and may be inset from ends of rod 730. An air gap (not shown) may separate two mu metal components (not shown) in rod 730. The air gap may be between about 0.5 in and about 2 in, such as about 1.5 in. The air gap may facilitate adjusting the direction of the magnetic field lines so that magnetic sensor 354 (FIG. 37) of foot support module 350 (FIGS. 36-37) may detect the magnetic field as it strengthens. Rod 730 may include mu metal 732 to alternately carry a stronger or weaker magnetic field based on proximity of magnets 706 to sidewall 720, and therefore mu metal conduit 722. In other words, when one or more magnets 706 are moved into proximity with mu metal conduit 722, a magnetic field is strengthened in mu metal conduit 722 and mu metal 732 in rod 730. Rod 730 may be circular in cross-section such that the magnetic field remains symmetrical during rotation of foot support 700, which may allow magnetic sensor 354 to maintain a sensitivity for detecting the magnetic field. Rod 730, or any other component having mu metal, from which magnetic sensor 354 detects the magnetic field, may also be termed a magnetic terminus. Mu metal components may be fully enclosed within stainless steel chambers to protect them from corrosion and cleaning processes, as mu-metal itself may be more susceptible to degradation. In embodiments, the one or more magnets strengthen the magnetic field of the mu metal conduct 722 across the stainless steel chamber and into the mu metal conduit 722. At the magnetic terminus, the magnetic field extends outside of the stainless steel rod enclosing the ends of the mu metal conduit. In one embodiment, the mu metal conduit is laid out in a generally straight line from the position opposite the one or more magnets 706 to the intersection with the rod 73

Foot support module 350 is shown in FIGS. 36-37 according to exemplary embodiments. In this embodiment, foot support module 350 may be part of positioning system 100. In another example, foot support module 350 may be a part of surgery table 200. In another example, foot support module 350 may function separately, such as with other positioning systems, surgery tables, or apparatuses thereof. In an example, foot support module 350 may include features that are functional separately and in combination.

Foot support module 350 may include a gripping mechanism 352. Gripping mechanism 352 may receive rod 730 of foot support 700 (FIG. 29). To retain rod 730, and therefore foot support 700, gripping mechanism 352 may rotate inward. To release rod 730, and therefore foot support 700, gripping mechanism 352 may be rotated outward, e.g., at least one side of gripping mechanism 342 rotating away from the other side. Foot support module 350 may include magnetic sensor 354, as shown in FIG. 37, to actuate gripping mechanism 352 when a magnetic field is present. Actuating gripping mechanism 352 may cause gripping mechanism 352 to rotate inward, e.g., at least one side of gripping mechanism 342 rotating toward the other side.

Foot support module 350 may also include a proximity sensor 355, as shown in FIG. 37. Proximity sensor 355 may detect metal proximate to gripping mechanism 352. If no metal is present, gripping mechanism 352 is prevented from rotating inward to secure around an object within gripping mechanism 352. If metal is detected, such as from rod 730, gripping mechanism 352 may be rotatable inward to grip the metal object. In this way, proximity sensor 355 may provide a safety feature to prevent injury from gripping user 40 (FIG. 19), for example.

Foot support module 350 may also include a button release 356, as shown in FIG. 36, to release gripping mechanism 352 such that gripping mechanism 352 is rotated outward and foot support 700 (FIG. 29) is not gripped. In this way, gripping mechanism 352 may be released by releasing user interface paddle 710 (FIG. 29), button release 356, or user interface paddle 710 and button release 356. Actuating button release 356 again may cause gripping mechanism 352 to rotate inward to grip foot support 700.

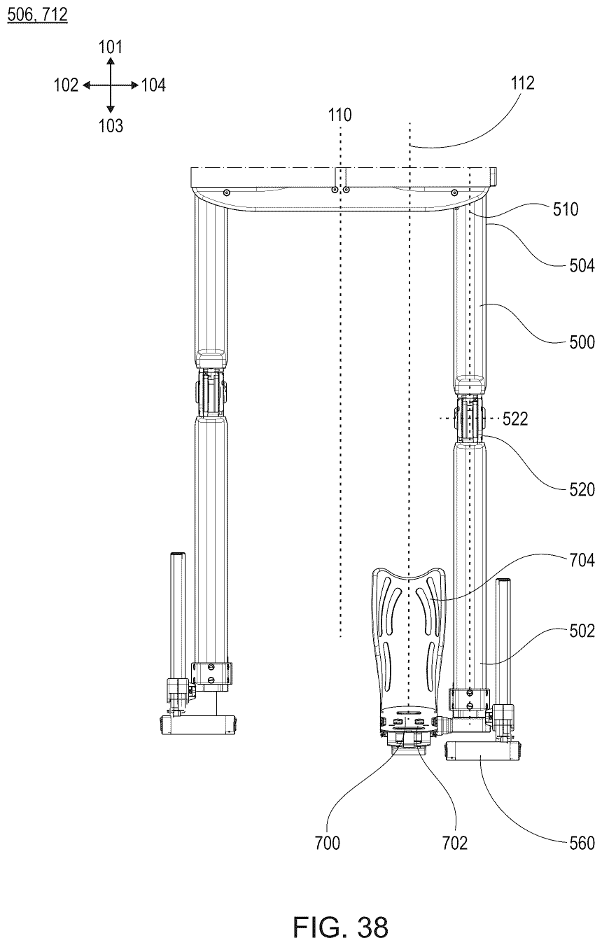

Foot support module 350 may also include a bellows 360 and a hinge 361. With reference to FIG. 38 according to exemplary embodiments, foot support module 350 may be rotatable inward. In other words, foot support module 350 may be moved in superior direction 102 and lateral direction 104. Foot support module 350 may be rotated inward when spar 500 is rotated outward. In other words, spar 500 may be moved laterally in a yaw motion by articulating second joint 426 (FIG. 14). Moving spar 500 laterally may cause leg 32 (FIG. 20) of patient 20 (FIG. 20) to move laterally. However, this movement could cause leg 32 to bend outwardly in the wrong direction, or for foot 36 (FIG. 20) to disengage from foot support 700. Accordingly, to accommodate yaw motion of spar 500 in lateral direction 104, foot support module 350 may rotate inward to prevent leg 32 of patient 20 from overstretching and foot 36 from disengaging from foot support 700. Bellows 360 and hinge 361 may provide yaw compensation for when spar 500 is moved laterally in a yaw motion. Bellows 360 may be expandable to allow for rotation of foot support module 350. As shown, hinge 361 may rotate against a bumper to act as a dampened flexure. Foot support module 350 may rotate inward up to about 20 degrees, such as up to about 15 degrees.

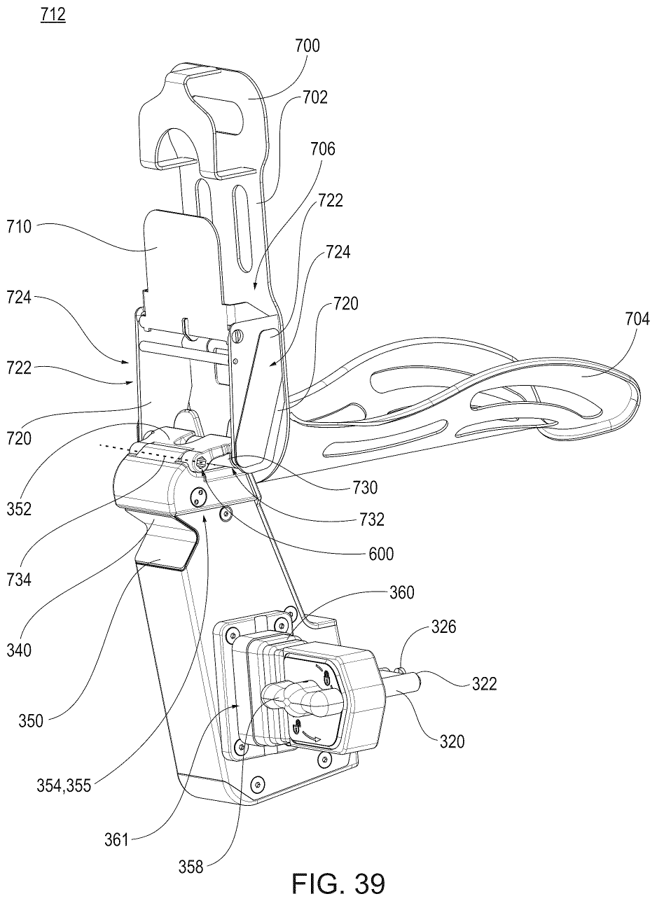

With reference to FIG. 39, foot support 700 may couple to foot support module 350. The coupling may be a mechanical coupling 600. Magnets 706 may actuate mechanical coupling 600. As foot support 700 may be in sterile space 10 (FIG. 20), magnets 706 may be disposed in sterile space 10. As foot support module 350 may be in working space 12 (FIG. 20), magnets 706 may generate a magnetic field that extends to foot support module 350 in working space 12 across one or more drapes 46 (FIG. 18) such that mechanical coupling 600 to couple foot support 700 and foot support module 350 may be actuated by magnets 706. Foot support 700 and foot support module 350 may be separated by one or more drapes 46, such as top drape 80 (FIG. 30), such as hard point 81 (FIG. 30) of top drape 80, and moving resiliently with gripping mechanism 352. Hard point 81 may be made of a rubber or polymer material that allows gripping mechanism 352 to grip and release rod 730 without breaking the sterile barrier formed by one or more drapes 46.

In embodiments, use of a magnet and mu metal to selectively propagate a magnetic field along a mu metal highway may be used in foot support 700, and may be used independently in other applications where it is useful to transmit such magnetic field from a magnet to a different location. In other embodiments, use of a magnet and a mu metal highway to extend a magnetic field across a barrier may be used in foot support 700 and foot support module 350 herein. Use of a magnetic and a mu metal highway to extend a magnetic field and may be used independently in other applications where it is useful to extend such magnetic field from a magnet to a different location, such as across a barrier such as cloth, plastic, rubber a wall, wood, and other like materials. Other applications may position the magnetic sensor to detect the magnetic field in another location, such as on a table or another surgical equipment. Other applications may be in alternative environments, such as athletic equipment, automotive, industrial, aerospace, other consumer products, or other medical devices.

Mechanical coupling 600 may be facilitated by gripping mechanism 352, for example. Mechanical coupling 600 may be actuated by magnetic sensor 354. Gripping mechanism 352 may be actuated by the magnetic field generated by one or more magnets 706. For example, mu metal 732 of rod 730 may be magnetized to actuate magnetic sensor 354 and cause gripping mechanism 352 to rotate inward. Gripping mechanism 352 may rotate inward when actuated to grip foot support 700, or retain rod 730, thereby actuating mechanical coupling 600 and securing foot support 700 to foot support module 350. Gripping mechanism 352 may rotate outward when released to release foot support 700 in the absence of a magnetic field. Gripping mechanism 352 may rotate outward to release rod 730 when magnetic sensor 354 is not actuated. Ultimately, actuation of the magnetic sensor 354 and gripping mechanism 352, both in the working space 12, may be accomplished by the user 40 actuating the user interface paddle 710 in the sterile space 10.

Foot support module 350 and foot support 700 may be coupled via mechanical coupling 600 such that foot support 700 may pitch about a rotational axis 734 relative to foot support module 350. Rod 730 may include rotational axis 734. Foot support 700 may rotate about rotational axis 734 when user interface paddle 710 is in extended position 712 and is not actuated, or when foot gripping mechanism 352 releases foot support 700. Foot support 700 may rotate to position foot 36 (FIG. 20) of patient 20 (FIG. 20).

With reference again to FIGS. 24-26, user interface paddle 710 may include retracted position 714. Retracted position 714 may result when user interface paddle 710 is actuated. Actuating user interface paddle 710 may release center joint 552 (FIG. 3) such that when user interface paddle 710 is in retracted position 714, foot support 700 may be secured by foot gripping mechanism 352, and spar 500 may be flexed to raise knee 36. When user interface paddle 710 is in retracted position 714, positioning system 100 may continue adjustment to third position 124 shown in FIGS. 26-29. In third position 124, spar base 400 may be in second position 408 between distal end 202 and proximal end 204 of surgery table 200 such that spar 500 which extends from spar base 400 is translated in superior direction 101. When user interface paddle 710 is in retracted position 714, positioning system 100 may substantially simultaneously fold distal end 502 of spar 500 and translate proximal end 504 of the spar 500 via spar base 400. In other words, spar 500 may flex and translate at the same time, or substantially simultaneously. Releasing user interface paddle 710 may halt motion of spar 500, or secure spar 500 in a particular orientation.

In certain embodiments (not shown), spar 500 may flex upward, or in the same direction as leg 32 of patient 20. In other words, joint 520 may be raised as leg 32 is flexed and knee 34 is raised.

In embodiments, when user interface paddle 170 is not actuated and gripping mechanism 352 is open, or rotated outward, rod 730 (FIG. 33) may rest inside open gripping mechanism 352, and user 40 may rotate the foot about rod 730 into any desirable position. User interface paddle 710 allows the user 40 to then actuate user interface paddle, grasp rod 730 with gripping mechanism 352, and then bend the knee of the patient 20 by combination of flex and translation. During this operation, user 40 may have access to patient's 20 knee from the medial 114, lateral 118, or inferior 116 position. In embodiments, user 40 may flex and translate the spar with one hand by gripping user interface paddle 710 of foot support 700.

Referring again to FIGS. 18-29, in embodiments, a method may include supporting at least a portion of leg 32 on spar 500. A magnetic field may be generated in sterile space 10, which may be on a first side of one or more drapes 46. The magnetic field may be generated by one or more magnets 706 (FIG. 33) of foot support 700 at distal end 502 of spar 500. The magnetic field may be extended to working space 12 on a second side of one or more drapes 46 opposing the first side. The magnetic field may be extended via mu metal included in foot support 700, such as mu metal 724 (FIG. 33) of sidewall 720 (FIG. 33) and mu metal 732 (FIG. 33) of rod 730 (FIG. 33). Magnetic sensor 354 (FIG. 37) may be actuated in working space 12. Accordingly, one or more magnets 706 and mu metal may be in sterile space 10. Magnetic sensor 354 may be in working space 12 such that the magnetic field is extended across one or more drapes 46. In other words, the magnetic field is remoted from sterile space 10 to working space 12. The mu metal may essentially form a magnetic highway that may remote the magnetic field across one or more drapes 46. Spar 500 may be flexed by releasing joint 520 (FIG. 3). Spar 500 may be moved relative to surgery table 200 to flex leg 32 of patient 20. Accordingly, inferior access to knee 34 of leg 32 may be allowed for user 40.

Another method may include supporting at least a portion of leg 32 on spar 500. Spar 500 may be flexed by releasing joint 520 (FIG. 3). Spar 500 may be moved relative to surgery table 200 to flex leg 32 of patient 20. Accordingly, inferior access to knee 34 of leg 32 may be allowed for user 40.

Foot support module 350 may be a table module 340 that may couple to surgery table 200, spar 500, or surgery table 200 and spar 500 to provide additional functionality for positioning system 100 (FIG. 1). Table modules 340 may include foot support module 350 and other modules, such as a distractor, femur hook, or a robotic arm. Table modules 340 may be apparatuses that couple to surgery table 200, spar 500, or surgery table 200 and spar 500 at several points. Accordingly, table modules 340 may be selectively positioned along surgery table 200, spar 500, or surgery table 200 and spar 500. The coupling between table modules 340 and surgery table 200, spar 500, or surgery table 200 and spar 500 may be a mechanical coupling, an electrical coupling, or a mechanical and electrical coupling. Table modules 340 may share coupling hardware such that each of foot support module 350, a femur hook module, a distractor, and a robotic arm module, for example, may couple to surgery table 200, spar 500, or surgery table 200 and spar 500 via the same coupling means.

Various universal attachment systems are shown with reference to FIGS. 36-41 according to exemplary embodiments. In an example, universal attachment systems may be part of positioning system 100. In another example, universal attachment systems may be a part of surgery table 200. In another example, controls may function separately, such as with other positioning systems, surgery tables, or apparatuses thereof. In an example, controls may include features that are functional separately and in combination.

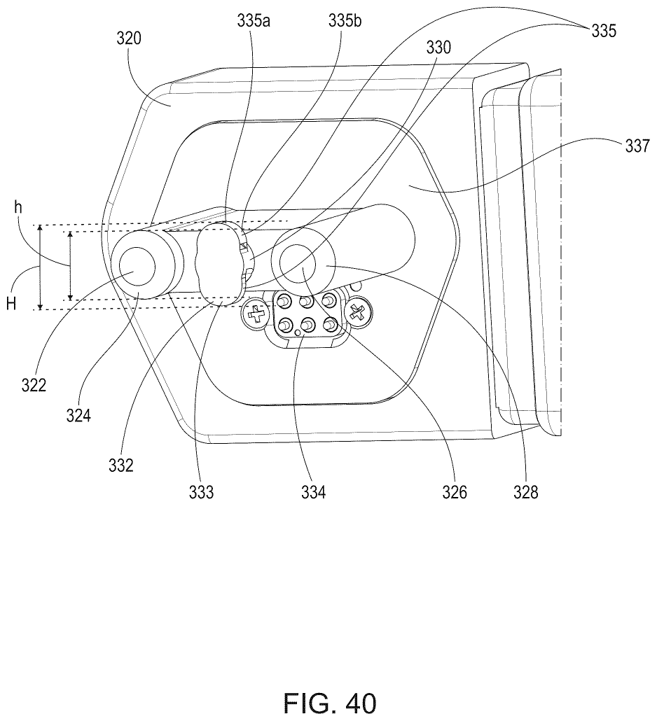

Foot support module 350 is shown in FIGS. 36-40 according to exemplary embodiments. In one embodiment, table modules 340, such as foot support module 350, may include one or more universal attachment interface (UAI) plugs 320. Referring to FIG. 40, in one embodiment, UAI plug 320 may include a “dog-bone” shape. For example, UAI plug 320 may include a first portion 322, a second portion 326, and a third portion 330 between first portion 322 and second portion 326. In some embodiments, first portion 322 and second portion 326 may be substantially spherical. In other embodiments, first portion 322 and second portion 326 may be oval, or polygonal. In an embodiment, third portion 330 may be substantially rectangular. In other embodiments, third portion 330 may include other shapes, for example, square, circular, oval, or polygonal. In certain embodiments, third portion 330 may have a height that is less than first portion 322 and/or second portion 326. Accordingly, in the embodiment shown, first portion 322, second portion 326, and third portion 330 may form a “dog-bone” shape. In embodiments where table module 340 is powered and/or communications with system 100 via a wired connection, UAI plug 320 may include an electrical plug 334.

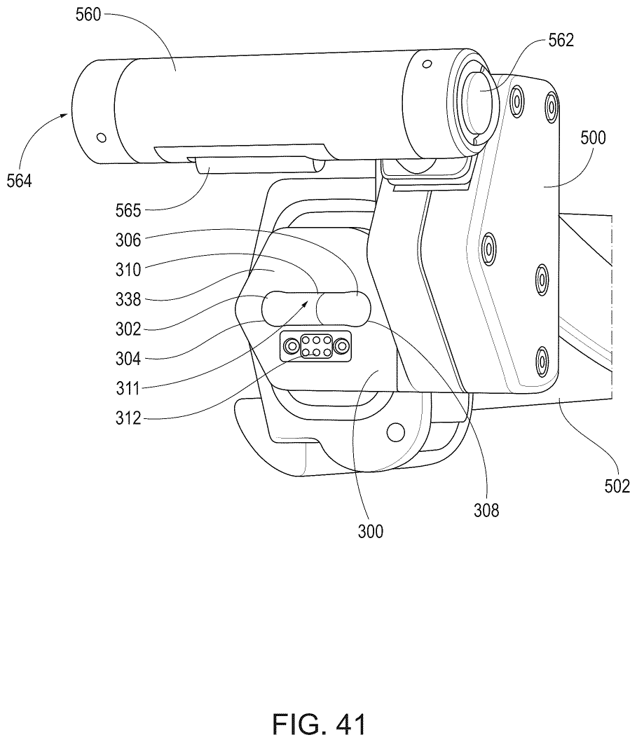

Referring still to FIG. 40, in embodiments, UAI plug 320 may include a locking portion 332. Third portion 330 may include a locking portion 334. Alternatively, locking portion 334 may extend from third portion 330. As shown in FIG. 40, locking portion 334 may be in a vertical position. Locking portion 332 may have a height and a width. In the vertical position, as shown, the height H of the locking portion 332 may be greater than height h of third portion 330. In a horizontal position, the height may be equal to or less than height h, and the width H may be less than a width of third portion 330. In other words, in a horizontal position, locking portion 332 may be sized to fit entirely within the profile of third portion 330 when viewed head-on, and when in vertical position, locking portion 332 may have a height H greater than height H of third portion 330. Locking portion 334 may be configured to rotate at least 90 degrees from a horizontal position to a vertical position to lock UAI plug 320 in UAI port 300 (FIG. 41). UAI plug 320 coupling to UAI port 300 will be discussed further below.

Positioning system 100 (FIG. 1) may include one or more UAI ports 300 as shown in FIG. 41 according to exemplary embodiments. UAI ports 300 may be universal attachments. Accordingly, UAI port 300 may receive two or more table modules 340, such as a femur hook or a robotic arm, a distractor, or foot support module 350. In other words, UAI port 300 may alternatively receive two or more table modules 340, each table module 340 being distinct from another of table modules 340. In some embodiments, UAI ports 300 may include a cutoff switch (not shown) to disengage table module 340 coupled to UAI port 300. Disengaging table module 340 may allow table module 340 to be removed from UAI port 300 and/or electrically disconnected. UAI ports 30 along positioning system 100 may be covered when not in use.

Referring to FIGS. 1 and 41, in various embodiments, surgery table 200, spar 500, or surgery table 200 and spar 500 may include one or more UAI ports 300. Distal end 502 of spar 500 may include one or more UAI ports 300. Distal end 402 of spar base 400 may include one or more UAI ports 300. Distal end 202 of surgery table 200 may include one or more UAI ports 300. In one embodiment, distal end 502 of spar 500 may include one or more UAI ports 300, distal end 402 of spar base 400 may include one or more UAI ports 300, and distal end 202 of surgery table 200 may include one or more UAI ports 300.

As shown in FIG. 41, spar 500 may include UAI port 300. For example, distal end 502 of spar 500 may include UAI port 300. With reference to the embodiment of FIG. 2, surgery table 200, such as platform 220, may include UAI port 300. For example, distal end 202 of surgery table 200 may include UAI port 300, such as along each lateral side, such as first side 206 (FIG. 1) and second side 208 (FIG. 1). In another embodiment, UAI port 300 may be disposed on a slider (not shown) that may translate along at least a portion of first side 206, for example. UAI port 300 may be disposed on a slider that may translate along at least a portion of second side 208, for example. Additionally or alternatively, UAI port 300 may be disposed on a slider that may translate out of platform 220, such as in lateral direction 104 (FIG. 1). The slider may translate in medial direction 102 (FIG. 1) back into platform 220. The slider may translate out of platform 220 up to about five inches, such as about three inches. The slider may translate out of platform 220 incrementally, such as in about one inch increments, such as in about half an inch increments. In this way, UAI port 300 may be selectively disposed on surgery table 200, such as platform 220, such as along first side 206, second side 208, or first side 206 and second side 208. In addition, the position of UAI port 300 may be adjusted according to patient 20 being on platform 220. For example, if patient 20 is larger, UAI port 300 may be located laterally from platform 220 to allow for table modules 340 (FIG. 1) to couple to UAI port 300 for user with patient 20. With reference to FIG. 9, spar base 400 may include UAI port 300. For example, UAI port 300 may be disposed between distal end 402 and proximal end 404 of spar base 400.

Referring again to the embodiment of FIG. 41, UAI port 300 may include a “dog-bone” shape. For example, UAI port 300 may include a first opening 302, a second opening 306, and a third opening 310 between first opening 302 and second opening 306. First opening 302 and second opening 306 may be substantially spherical. Third opening 310 may be substantially rectangular. Accordingly, first opening 302, second opening 306, and third opening 310 may form a “dog-bone” shape. In other embodiments, first opening 302 and second opening 306 may be oval, or polygonal. In other embodiments, third opening 310 may include other shapes, for example, square, circular, oval, or polygonal. In embodiments, first opening 302 and second opening 306 and third opening 310 correspond to the shapes of UAI plug first portion 322, second portion 326, and third portion 330. UAI port 300 may also include an electrical connection 312 to receive electrical plug 334 (FIG. 40) of UAI plug 320. Accordingly, table module 340 (FIG. 39) coupled to UAI port 300 may be electrically coupled to UAI port 300.

Referring to FIGS. 36, 40-41, in embodiments, UAI port 300 may include an undercut 311 inside of third opening 310 and corresponding to locking portion 332 in the position shown in FIGS. 36 and 40. Undercut 311 may be sized such that locking portion 334 may enter in the horizontal position, but may not be able to be removed in the vertical position. Undercut 311 may have an undercut width that is equal to or greater than the width of the locking portion 332 in FIGS. 36 and 40, and an undercut height that is less than the height H of locking portion 332. When locking portion 332 is inserted into undercut 311 and rotated to the vertical position, the width of locking portion 332(now vertical) is now greater than the height of third opening 310 and corresponding undercut height, preventing locking portion 332, and correspondingly, UAI plug 320, from being removed from UAI port 300.

In some embodiments, UAI plug 320 and UAI port 300 include a twist lock. In embodiments, UAI plug 320 may include a locking portion 332 (FIGS. 36, 40). Locking portion 332 may have a height and a width, wherein, when in a horizontal position (90 degrees from what is shown in FIGS. 36, 40), the height may be equal to or less than h, and the width may be less than a width of third portion 330. Locking portion 332 may be configured to rotate at least 90 degrees from a horizontal position to a vertical position as shown in FIGS. 356, 40. In the vertical position, the width of the locking portion 332 is now oriented vertically and may have a height H, and H may greater than height h of third portion 330. In embodiments, UAI port 300 includes an undercut 311 inside of third opening 310 and corresponding to locking portion 332. Undercut 311 may be sized such that locking portion 332 may enter in the horizontal position where the height of locking portion 332 is height h, but may not be able to be removed in the vertical position where the locking portion 332 is now oriented vertically and may have a height H. Undercut 311 may have an undercut width that is equal to or greater than the width of the locking portion 332 in a horizontal position, and an undercut height that is equal to or greater than height H of locking portion 332 oriented vertically.

When locking portion 332 is inserted into undercut 311 and rotated to the vertical position, height of locking portion 332 is now greater than the height of third opening 310, preventing locking portion 332 from being removed from undercut 311, and correspondingly, preventing UAI plug 320, from being removed from UAI port 300.

In embodiments, a proximal face 336 is opposite distal face 333 of locking portion 332, and in the horizontal position proximal face 336 may include one or more ramped surfaces 335, on sides of proximal face 336, such as one side shown in FIG. 36 and FIG. 40 comprising respective lower portion 335a and upper portions 335b. When locking portion 332 is rotated towards lower portions 335a (counterclockwise in the embodiment of FIGS. 36, 40), proximal face 335 may engage a proximal wall of undercut 311 beginning at lower portion 335a and moving to upper portion 335b. As locking portion 332 is rotated, ramped surfaces 335 may ride along proximal wall of undercut 311 and may pull the UAI plug 320 any remaining distance into UAI port 300, and then increase retention force of UAI plug 320 in UAI port 300. In one embodiment, UAI port face 337 is pulled by locking portion 332 against a corresponding UAI port face 338 of UAI port 300, providing a secure connection between UAI plug 320 and UAI port 300. In order to release UAI plug 320 from UAI port 300, locking portion 332 may be rotated in the opposite direction back to the horizontal position. UAI port 300 may receive UAI plugs 320 of table modules 340, with reference to FIGS. 40-41. In this way, table module 340 may couple to surgery table 200, spar 500, or surgery table 200 and spar 500. First opening 302 of UAI port 300 may receive first portion 322 of UAI plug 320. Second opening 306 of UAI port 300 may receive second portion 326 of UAI plug 320. Third opening 310 of UAI port 300 may receive third portion 330 of UAI plug 320. Table module 340 may include a UAI lock 358 to retain UAI plug 320 in UAI port 300. UAI lock 358 may be unique to each individual table module 340, however, each UAI lock 358 functions to rotate locking portion 332 from a horizontal position to a vertical position. UAI lock 358 may be a lever to secure UAI plug 320 in UAI port 300. UAI lock 358 may turn locking portion 332 such that locking portion 332 is turned withing undercut 311, and therefore UAI plug 320 is secured withing UAI port 300.

First portion 322 and second portion 326 may guide UAI plug 320 such that UAI port 300 may receive table module 340. First portion 322 may include a shape to guide UAI plug 320. For example, first portion 322 may include a bevel shape 324 to guide UAI plug 320 such that UAI port 300 may receive table module 340. In addition, second portion 326 may include a shape to guide UAI plug 320. For example, second portion 326 may include a bevel shape 328 to guide UAI plug 320 such that UAI port 300 may receive table module 340. Similarly, first opening 302 and second opening 306 may guide UAI plug 320 of table module 340 such that UAI port 300 may receive table module 340. For example, first opening 302 and second opening 306 may include a bevel shape 304 and a bevel shape 308, respectively, to guide UAI plug 320 such that UAI port 300 may receive table module 340. Respective bevel shapes may help to receive and center UAI plug 320 in UAI port 300. However, once UAI plug 320 is inserted into UAI port 300 beyond the engagement of respective bevel shapes, first opening 302 and second opening 306 and third opening 310 securely engage corresponding UAI plug first portion 322, second portion 326, and third portion 330, and UAI port face 337 being pulled by locking portion 332 against a corresponding UAI plug face 338 of UAI port 300 additionally secures connection between UAI plug 320 and UAI port 300.

An apparatus may include UAI port 300 and UAI plug 320, such as a UAI attachment system. In an example, UAI port 300 and UAI plug 320 may be part of positioning system 100. In another example, UAI port 300 and UAI plug 320 may be a part of surgery table 200, spar 500, or spar base 200. In another example, UAI port 300 and UAI plug 320 may function separately, such as with other positioning systems, surgery tables, or apparatuses thereof. In an example, UAI port 300 and UAI plug 320 may include features that are functional separately and in combination.

In an example, an attachment system for a surgical procedure may include a UAI port 300. UAI port 300 may be configured to alternatively receive two or more table modules 340, each table module 340 being distinct from another of table modules 340. The attachment system may also include a UAI plug 320. Each table module 340 may include one or more UAI plugs 320.

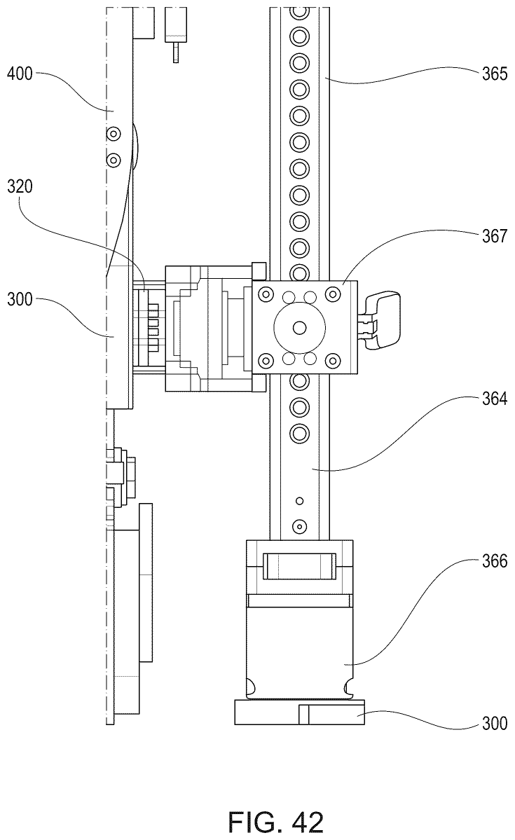

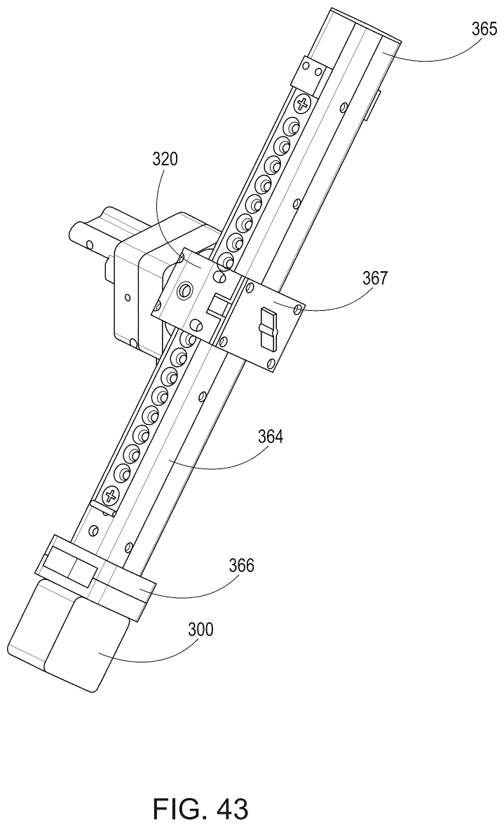

With reference to FIGS. 40-43, various attachments for positioning system 100 (FIG. 1), such as surgery table 200 (FIG. 1) or spar 500 (FIG. 3), or components thereof, may include UAI port 300, UAI plug 320, or UAI port 300 and UAI plug 320. In one embodiment, as shown in FIGS. 42-43 according to exemplary embodiments, positioning system 100 may include a UAI offset arm 364. UAI offset arm 364 may be disposed along positioning system 100, such as on surgery table 200 or spar 500, or components thereof. UAI offset arm 364 may be selectively disposed.

In this embodiment, UAI offset arm 364 may include an arm 364 having a proximal end 365 and a distal end 366 opposing proximal end 365. Distal end 366 may include UAI port 300. Proximal end 365 may include UAI plug 320. Alternatively, UAI plug 320 may be disposed intermediate to proximal end 365 and distal end 366. UAI plug 320 may be coupled to a bracket 367 within which arm 364 may be adjustable to change the position of proximal end 365 and distal end 366. For example, arm 364 may slide within bracket 367 to further extend distal end 366 beyond bracket 367. UAI plug 320 of UAI offset arm 364 may couple to UAI port 300 of positioning system 100, such as on surgery table 200, spar 500, spar base 400, or components thereof, such that UAI offset arm 364 is selectively disposed. For example, UAI plug 320 of UAI offset arm 364 may couple to UAI port 300 of spar base 400. UAI offset arm 364 may be selectively disposed at any UAI port 300. Arm 364 may be adjustable within bracket 367 to further extend proximally UAI port 300. In addition, arm 364 may be articulable in a pitch direction. UAI offset arm 364 rotated may be seen in FIG. 43. UAI offset arm 364 may pitch upward in anterior direction 105 (FIG. 1) or posterior direction 107 (FIG. 1) to about 30 degrees, such as up to about 20 degrees, such as up to about 10 degrees. In this way, attachments, such as table modules 340 (FIG. 1) with UAI plugs 320 may couple to UAI port 300 of UAI offset arm 364 and may be selectively disposed along positioning system 100 (FIG. 1), such as surgery table 200 (FIG. 1) or spar 500 (FIG. 3) or spar base 300, or components thereof. UAI offset arm 364 may allow the reach of attachments to be extended and adjustable.

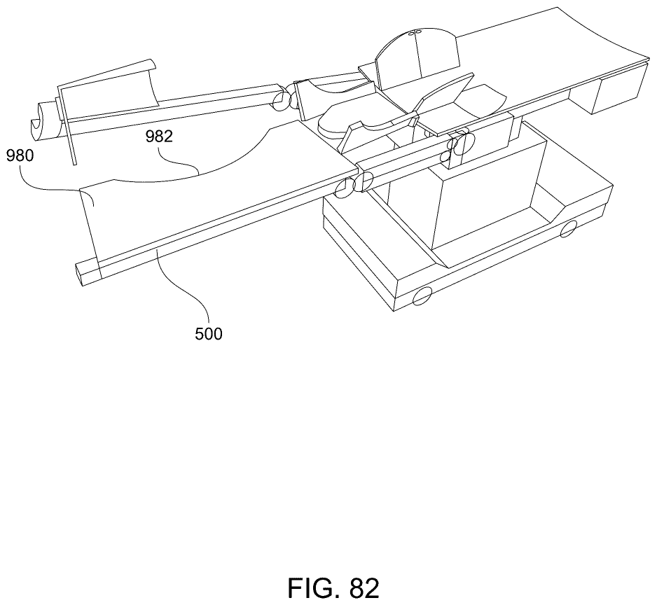

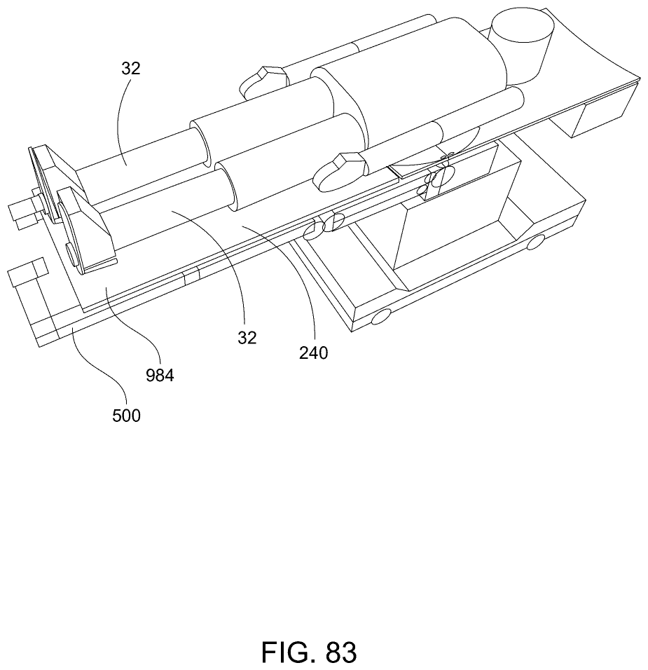

In embodiments, a portable UAI 368 (not shown) may additionally or alternatively be disposed along positioning system 100 (FIG. 1), such as surgery table 200 (FIG. 1) or spar 500 (FIG. 3) or rail 226, or components thereof. In one embodiment, portable UAI 368 may have a smaller profile, or may be more compact, in comparison to UAI offset arm 364. Like UAI offset arm 364, portable UAI 364 may be disposed along positioning system 100, such as on surgery table 200 or spar 500, or components thereof. Portable UAI 364 may be selectively disposed. For example, portable UAI 368 may have a clamp to couple to a portion of positioning system 100, such as surgery table 200, spar 500, or components thereof. Portable UAI 368 may mount to rail 226 (FIG. 1), spar 500, or one or more edges of boards that may attach to positioning system 100, such as on surgery table 200 or spar 500, or components thereof, and may be slideable before being fixed in position. Portable UAI 368 may be side-mounted to spar 500, or one or more edges of boards, such as a lateral board 980 (FIG. 82) or an imaging board 984 (FIG. 83). Portable UAI 368 may have an adapter to couple to different components. Accordingly, portable UAI 368 may couple to positioning system 100, such as on surgery table 200 or spar 500, or components thereof, such that portable UAI 368 is selectively disposed. Portable UAI 368 may break imageability where attached, e.g., by having an aluminum, or other metal, adapter.

Portable UAI 368 may include a UAI port 300. In this way, attachments, such as table modules 340 (FIG. 1) with UAI plugs 320 may couple to UAI port 300 of portable UAI 368 and may be selectively disposed along positioning system 100, such as surgery table 200 or spar 500, or components thereof. Portable UAI 368 may be selectively coupled where UAI offset arm 364 or other components with UAI ports 300 do not reach such that portable UAI 368 may extend coverage of UAI ports 300 for receiving table modules 340.

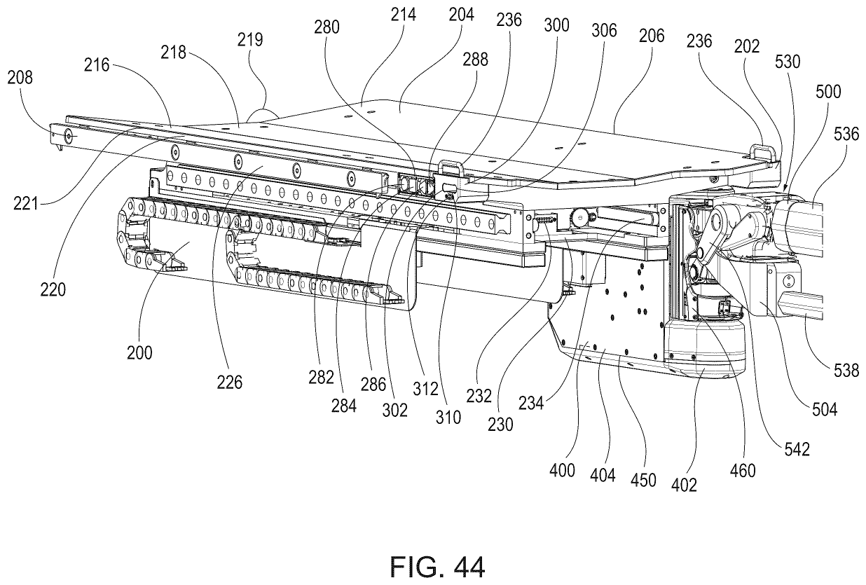

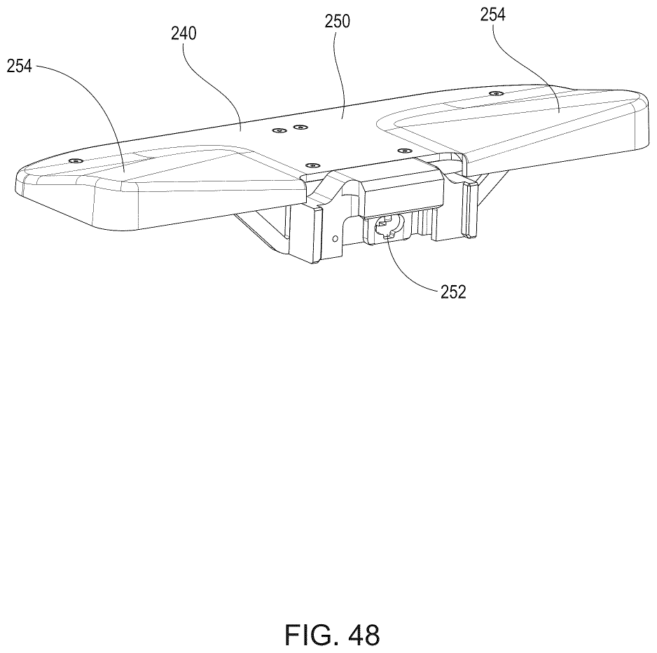

Positioning system 100 may (FIG. 1) may include one or more additional universal interfaces in that a plurality of attachments may be coupled to the same component. With reference to FIGS. 44-46, according to exemplary embodiments, positioning system 100, such as surgery table 200 may include a pelvic port 230. Pelvic port 230 may be disposed at distal end 202 of surgery table 200, for example. Accordingly, pelvic port 230 may be proximate a pelvic area of patient 20 (FIG. 20) Pelvic port 230 may receive a plurality of attachments 240 (FIG. 48) to support at least a portion of patient 20. In this way, pelvic port 230 may be a universal receptacle as it may receive a plurality of distinct attachments 240.

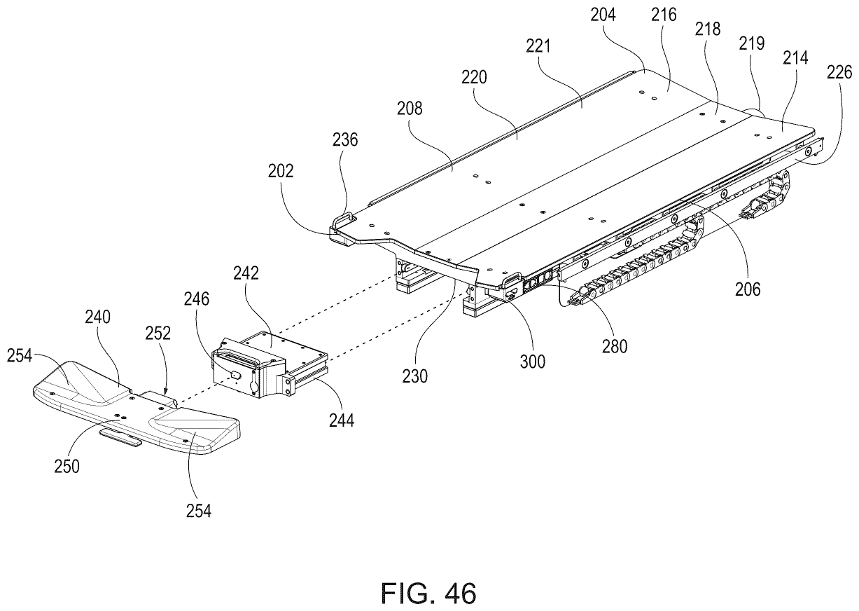

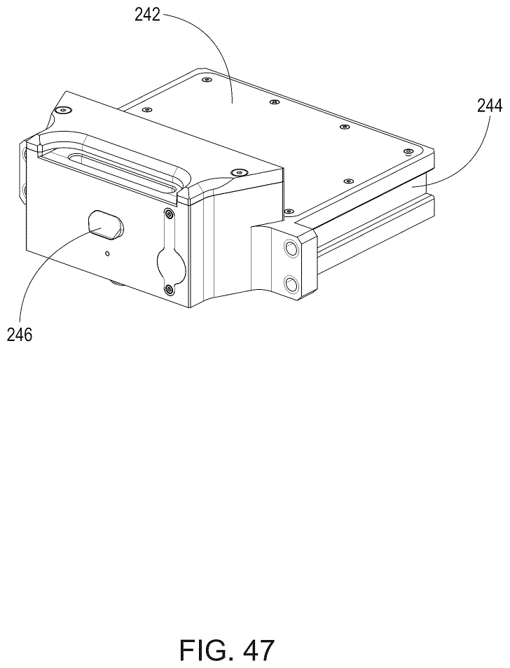

Pelvic port 230 may directly receive attachments 240 (FIG. 48). Additionally or alternatively, with reference to FIGS. 46-47 according to exemplary embodiments, pelvic port 230 may receive a pelvic port adapter 242. Pelvic port adapter 242 may receive attachments 240. Pelvic port 230 may include an electrical coupling 232 to couple to attachments 240 or pelvic port adapter 242. In addition, pelvic port 230 may include one or more rails 234. Pelvic port adapter 242 may include a track 244. Track 244 may receive one or more rails 234 such that pelvic port adapter 242 is coupled to pelvic port 230.

Attachments 240 may include supports for at least a portion of patient 20 (FIG. 20), such as at least a portion of a lower body of patient 20. An example attachment 240 is shown in FIG. 48. Attachment 240 may be a knee ledge board 250. Knee ledge board 250 may support at least a portion of patient 20, such as an at least a pelvic area of patient 20. In embodiments, knee ledge board 250 may be radiolucent. In other embodiments, knee ledge board 250 may include a portion that is substantially radiolucent. Alternatively, knee ledge board 250 may be mostly or entirely radiolucent. For example, knee ledge board 250 may consist essentially of carbon fiber, except for a mounting portion that attaches knee ledge board 250 to pelvic port 230 or pelvic port adapter 240. In other words, knee ledge board 250 may be composed mostly or entirely of carbon fiber. In this way, knee ledge board 250 may be radiolucent and/or substantially radio-uniform, as carbon fiber is radiolucent material.

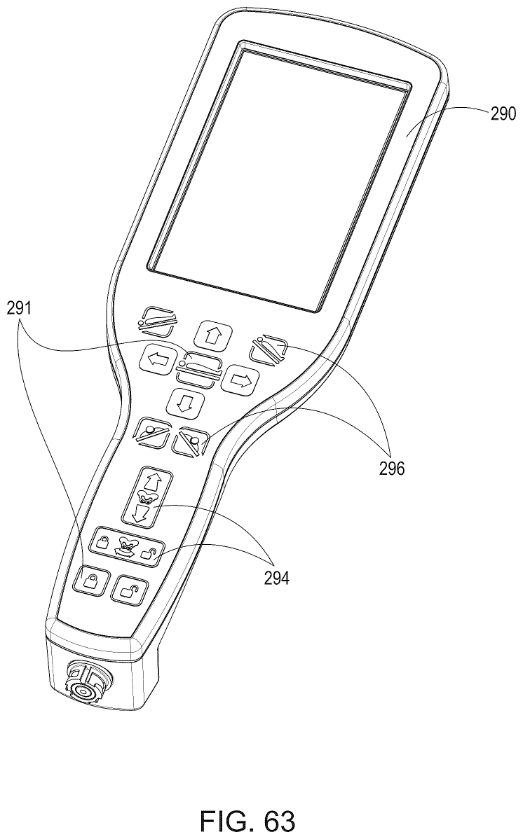

Pelvic port adapter 242 shown in FIG. 47 may include a protrusion 246. As shown in FIG. 48 according to exemplary embodiments, attachments 240, such as knee ledge board 250, may include a receptacle 252 to receive protrusion 246 of pelvic port adapter 242. Attachments 240 with varying structures may include a common coupling with pelvic port 230 to couple to pelvic port 230. Pelvic port adapter 242 may allow for static and articulable attachments 240 to couple to pelvic port 230. For example, electrical coupling 232 may articulate pelvic port adapter 242 such that the attachments 240 may be articulable. Additionally or alternatively, attachments 240 may be static and not articulable. One or more controls may be proximate to pelvic port adapter 242 or remote from pelvic center board 840 to control articulation of pelvic port adapter 242. For example, a pendant 290 (FIG. 63) may include one or more controls 294 (FIG. 63) for articulating pelvic port adapter 242 and therefore attachments 240 coupled to pelvic port adapter 242. In embodiments, pelvic port adapter 242 may include powered movement for an attachment 240 in at least one or more of pitch, tilt, and yaw directions.

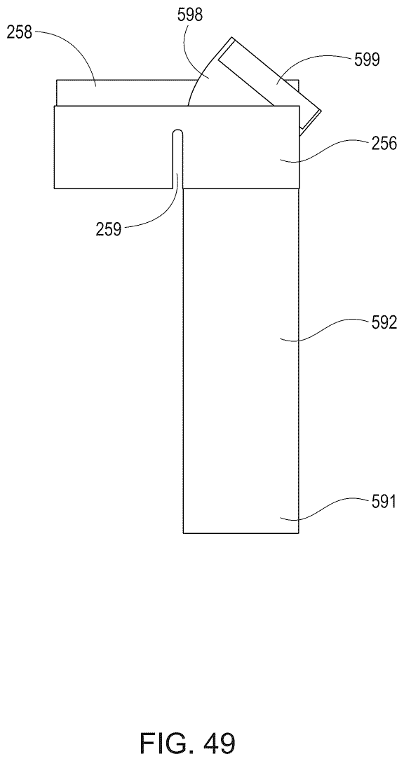

In embodiments, knee ledge board 250 may include one or more grooves 254 to support at least a portion of patient 20 (FIG. 20). Knee ledge board 250 may receive one or more pads to support at least a portion of patient 20, such as knee ledge board pad 256 shown in FIG. 49 according to exemplary embodiments. Knee ledge board pad 256 may include a flexible, cushioning material. Knee ledge board pad 256 therefore may provide support and prevent pressure injuries. Knee ledge board pad 256 may include an anchor 258 to couple to trunk pad 260 (FIG. 1). Anchor 258 may be a fastener such as a hook and loop fastener. Additionally or alternatively, anchor 258 may include another fastener, such as adhesive, a button clasp, or a magnetic attachment. The fastener may facilitate attachment to trunk pad 260.

Another attachment 240 may be imaging board 984, shown in FIG. 83 according to exemplary embodiments. Imaging board 984 may be coupled to pelvic port 230. Imaging board 984 may be static and not articulable. Imaging board 984 may be an attachment 240 and may couple to and extend from pelvic port 230 to distal end 502 (FIG. 1) of spars 500 (FIG. 1). Alternatively, imaging board 984 may extend from pelvic port 230 to above a part of spars 500 between proximal end 504 (FIG. 1) and distal end 502. Imaging board 984 may support at least a portion of patient 20 (FIG. 17). Spars 500 may extend under at least a portion of imaging board 984 and may not be coupled to the imaging board. Accordingly, spars 500 may be articulable under imaging board 984. In this way, imaging board 984 may function as a “diving board,” with spars 500 traveling underneath for maximum imaging flexibility. User 40 (FIG. 19) may use imaging board 984 for imaging while minimizing or eliminating interference from spars 500. While using imaging board 984, spars 500 may still be used to secure the foot of a patient 20 by any of the methods described herein. Alternatively, imaging board 984 may be coupled to surgery table 200 (FIG. 1) via pelvic port 230 and spars 500 may be removed.

Imaging board 984 may span about a full width of surgery table 200 (FIG. 1). Edges of imaging board 984 may be engineered to minimize risk of pressure injury. Imaging board 984 may be radiolucent. Imaging board 984 may include carbon fiber.

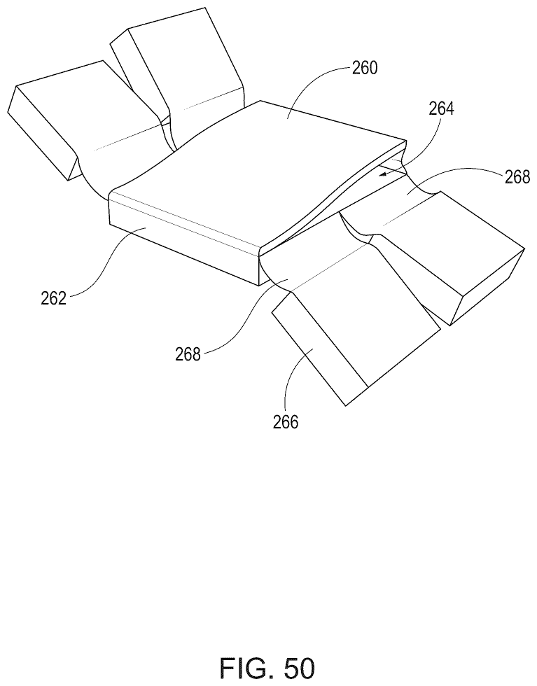

Trunk pad 260 is shown in FIG. 50 according to exemplary embodiments. As discussed, trunk pad 260 may be disposed along platform 220 (FIG. 1) of surgery table 200 (FIG. 1). Trunk pad 260 may support at least a portion of patient 20 (FIG. 20), such as at least a torso 26 (FIG. 20) of patient 20. Trunk pad 260 may include a flexible, cushioning material. Trunk pad 260 therefore may provide support and prevent pressure injuries. Trunk pad 260 may include a center pad 262. Center pad 262 may be disposed under at least a portion of torso 26. Trunk pad 260 may also include an opening 264. Opening 264 may be formed in center pad 262. Opening 264 may receive an insert, or a cushion, to provide additional support to at least a portion of patient 20. For example, patient 20 may be positioned laterally and may require additional support along a waist area or a back area. The insert may be added through opening 264 to center portion 262 to provide additional support to patient 20 along these areas. Inserts therefore may provide support and prevent pressure injuries. Inserts may be generally wedge shaped, rounded, polygonal, or any other shape, for example.

Trunk pad 260 may also include one or more lateral pads 266. Lateral pads 266 may extend from center pad 262 in lateral direction 104 (FIG. 1), for example. Lateral pads 266 may support at least a portion of patient 20. For example, at least a portion of patient 20 may extend laterally from center pad 262. Lateral pads 266 may provide support to patient 20 accordingly. Trunk pad 260 may also include one or more hammock attachments 268. Lateral pads 266 may be coupled to center pad 262 via hammock attachments 268. Each hammock attachment 268 may include a length of flexible material to adjust the extension of the respective lateral pad 266 relative to center pad 262 in medial direction 102 (FIG. 1) and lateral direction 104. For example, lateral pad 266 may extend further laterally. Surgery table 200 may include about two lateral pads 266 along each lateral side, such as first side 206 (FIG. 1) and second side 208 (FIG. 1). Alternatively, surgery table 200 may include about three lateral pads 266 along each lateral side, such as first side 206 (FIG. 1) and second side 208 (FIG. 1). Each lateral pad 266 may be individually adjustable separate from other lateral pads 266.

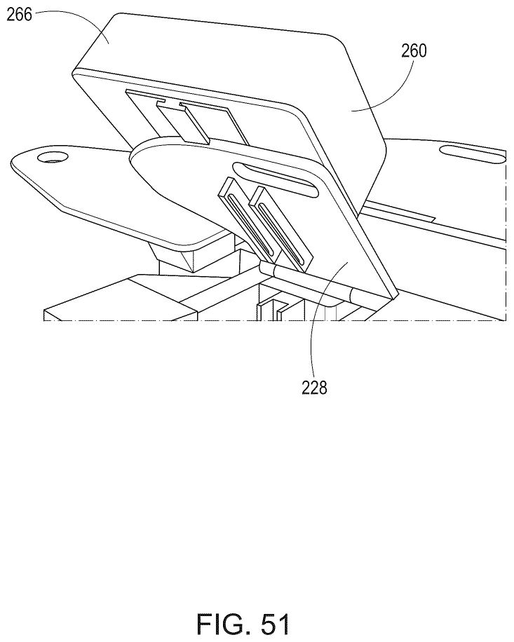

Surgery table 200 (FIG. 1) may include one or more components to receive trunk pad 260. Platform 220 (FIG. 1), for example, may receive at least a portion of trunk pad 260, such as center pad 262. Surgery table 200 may also include one or more lateral flaps 228, shown in FIG. 51 according to exemplary embodiments. Lateral flaps 228 may extend from platform 220 in lateral direction 104 (FIG. 1). Alternatively, lateral flaps 228 may be formed as part of platform 220 such that lateral flaps 228 replace first static flap 214 (FIG. 2) and second static flap 216 (FIG. 2). Lateral flaps 228 may receive lateral pads 266. Lateral flaps 228 may be translatable and rotatable. Lateral pads 266 may be translatable and rotatable with lateral flaps 228. Lateral flaps 228 may each be independently articulable.

Lateral flaps 228 may be translatable such that the position of each lateral flap 228 relative to platform 220 is adjustable in medial direction 102 (FIG. 1) and lateral direction 104 (FIG. 1). For example, lateral flaps 228 may translate out of platform 220, such as in lateral direction 104. Lateral flaps 228 may translate in medial direction 102 back into platform 220. Lateral flaps 228 may translate out of platform 220 up to about five inches, such as about four inches. Lateral flaps 228 may translate out of platform 220 incrementally, such as in about one inch increments, such as in about half an inch increments.

In addition, lateral flaps 228 may be rotatable. Accordingly, the pitch of lateral flaps 228 relative to platform 220 may be adjustable. In other words, lateral flaps 228 may be rotatable such that the position of each lateral flap 228 relative to platform 220 is adjustable in anterior direction 105 (FIG. 1) and posterior direction 107 (FIG. 1). Lateral flaps 228 may be rotatable continuously. Additionally or alternatively, lateral flaps 228 may be rotatable incrementally. Lateral flaps 228 may pitch in anterior direction 105 up to about 90 degrees, such as up to about 75 degrees. Lateral flaps 228 may pitch in anterior direction 105 incrementally, such as between about three degree and about eight degree increments, such as between about four degree and about six degree increments, such as in about five degree increments.

Lateral flaps 228 may be translatable and rotatable via a single control, or user interface, and configured such that a user may manipulate the single control with a single hand. In this way, lateral flaps 228 may be selectively disposed on surgery table 200, such as platform 220. In addition, the position of lateral flaps 228 may be adjusted according to patient 20 being on platform 220. For example, if patient 20 is larger, lateral flaps 228 may be located laterally from platform 220 to support patient 20.

Surgery table 200 may include about two lateral flaps 228 along each lateral side, such as first side 206 (FIG. 1) and second side 208 (FIG. 1). Alternatively, surgery table 200 may include about three lateral flaps 228 along each lateral side, such as first side 206 (FIG. 1) and second side 208 (FIG. 1). Each lateral flap 228 may be individually adjustable separate from other lateral flaps 220.

Referring to FIGS. 50-51, in one embodiment, when patient 20 (FIG. 20) is supine, all lateral flaps 228 may be lowered all of the way. In other embodiments, when patient 20 is supine, all lateral flaps 228 may be raised at some increment in order to cradle the body of patient 20. Lateral flaps 228 may be adjusted in medial direction 102 (FIG. 1) and lateral direction 104 (FIG. 1) to accommodate different sized patients 20. For example, lateral flaps 228 may be adjusted to translate laterally to accommodate larger sized patients. In other embodiments, when patient 20 is prone, all lateral flaps 228 may be lowered all of the way. In other embodiments, when patient 20 is prone, all lateral flaps 228 may be raised at some increment in order to cradle the body of patient 20, and lateral flaps 228 may be adjusted in the medial direction 102 and lateral direction 104 to accommodate different sized patients. In still further embodiments when patient 20 is prone, lateral flaps 228 may be raised at some increment in order to cradle the body of patient 20, and used in combination with trunk pads 260 and hammock attachment 268. In still further embodiments, when patient 20 is lying laterally, one side of lateral flaps 228 may be raised, and in some embodiments to a maximum height, to support the back of patient 20, while the other side of lateral flaps 228 may be lowered, or alternatively, also raised to support the front side of patient 20. In another embodiment, for patient 20 laying laterally, all lateral flaps 228 along the backside of patient 20 may be raised to a substantially maximum height, and/or lateral flaps 228 towards inferior direction 103 (FIG. 1) of the table may also be raised to a substantially maximum height, and one or more flaps towards superior direction 103 (FIG. 1) of surgery table 200 (FIG. 1) may be lowered completely to provide clearance for arms 22 (FIG. 20) of patient 20. In any position, the proximal most lateral flap 228 may be lowered to accommodate one or more arms 22 (FIG. 20) of patient 20.

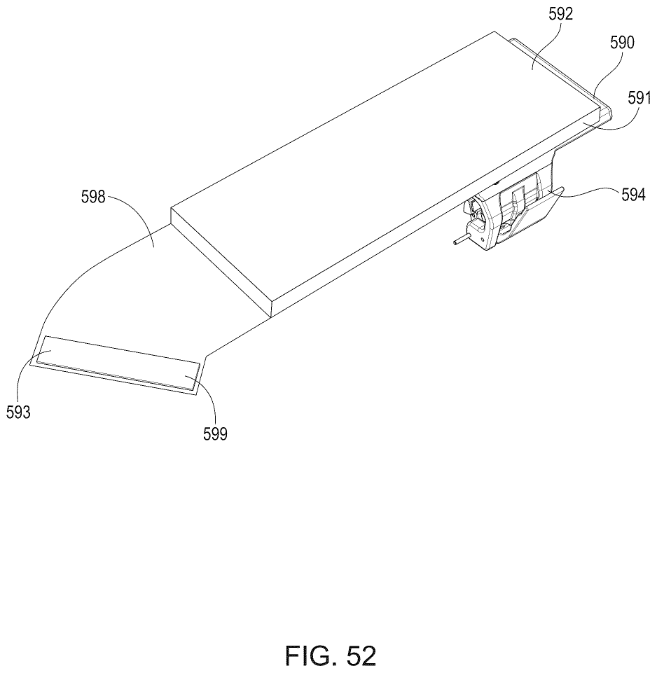

Referring to FIGS. 49-50, knee ledge board pad 256 may be coupled to trunk pad 260. In addition, knee ledge board pad 256 (FIG. 49) may be coupled to one or more spar board pads 592 shown in FIG. 52 according to exemplary embodiments. Each spar board pad 590 may be disposed over a spar 500 (FIG. 3). Spar board pads 592 may support at least a portion of patient 20 (FIG. 20), such as at least a portion of a leg (FIG. 20) of patient 20. Spar pads 592 may include a flexible, cushioning material. Spar board pad 592 therefore may provide support and prevent pressure injuries. Spar board pad 592 may include an anchor 599 to couple to knee ledge board pad 256. Anchor 599 may be a fastener such as a hook and loop fastener. Additionally or alternatively, anchor 599 may include another fastener, such as adhesive, a button clasp, or a magnetic attachment. The fastener may facilitate attachment to knee ledge board pad 256.

Knee ledge board pad 256 may also include a cutout 259. Cutout 259 may be an opening, or a slit, through a center of knee ledge board pad 256 between spar board pads 592. Cutout 259 may provide flexibility to knee ledge board pad 256. Accordingly, as spars 500 (FIG. 1) may be separated to allow user 40 (FIG. 19) medial access to knee 34 (FIG. 20), spars 500 may be moved laterally in a yaw motion. Second joint 426 (FIG. 14) may be disposed below knee ledge board 250 (FIG. 48) such that spars 500 move relative to knee ledge board 250 and knee ledge board pad 256. Additionally, spars 500 may be moved in anterior direction 105 (FIG. 1) or posterior direction 107 (FIG. 1) in a pitch motion. First joint 422 (FIG. 13) may be disposed below knee ledge board 250 such that spars 500 move relative to knee ledge board 250 and knee ledge board pad 256. Accordingly, spar board pads 592 disposed over spars 500 move relative to knee ledge board 250 and knee ledge board pad 256. Cutout 259 allows knee ledge board pad 256 to accommodate the movement of spar board pads 592, which are coupled to knee ledge board pad 256. In this way, knee ledge board pad 256 does not shift off of knee ledge board 250 or tear when spar board pads 592 are moved as spars 500 are moved.

Spar board pad 592 may be disposed on at least a portion of a spar board 590, as shown in FIG. 52. Spar board 590 is shown in FIGS. 52-54 according to exemplary embodiments. Spar boards 590 may be radiolucent. Spar boards 590 may include a portion that is substantially radiolucent. Alternatively, spar boards 590 may be mostly or entirely radiolucent. For example, spar boards 590 may consist essentially of carbon fiber. In other words, spar boards 590 may be composed mostly or entirely of carbon fiber. In this way, spar boards 590 may be radiolucent as carbon fiber is radiolucent material.

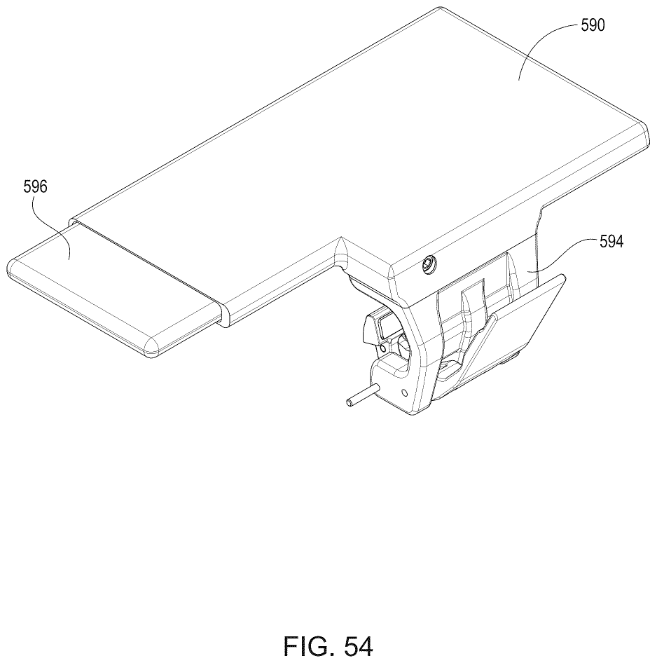

Spar board 590 may be disposed over a spar 500 (FIG. 3). Spar board 590 may support at least a portion of patient 20 (FIG. 20), such as at least a portion of a leg (FIG. 20) of patient 20. For example, spar boards 590 may be coupled to distal end 502 (FIG. 3) of spar 500, proximal end 504 (FIG. 3) of spar 500, or distal end 502 and proximal end 504. In an example, spar boards 590 may be coupled to distal end 502 of spars 500. Spar boards 590 may couple to spars 500 from lateral direction 104 (FIG. 1), medial direction 102 (FIG. 1), or lateral direction 104 and medial direction 102. Referring to FIG. 54, spar board 592 may include a clamp 594. Clamp 594 may couple spar board 592 to spar 500. Clamp 594 may couple spar board 592 from lateral direction 104 (FIG. 1), medial direction 102 (FIG. 1), or lateral direction 104 and medial direction 102. Clamp 594 may include a profile corresponding to, or similar to, a cross-section of spar 500. For example, spar 500 may include a cross-section having a substantially hexagonal shape. Clamp 594 may include a profile having a substantially hexagonal shape. In this way, clamp 594 may fit securely around spar 500.

Referring to FIGS. 52 and 54, spar board 590 may include an extension 596. Extension 596 may extend and retract relative to spar board 590 to adjust the length of spar board 590. In this way, spar board 590 may be adapted for different sized patients who may be taller or shorter. In addition, spar board 590 may provide support to the patient as the patient's leg is extended to mimic support provided by a traditional surgery table. Spar board pad 592 may be disposed on at least a portion of spar board 590. Spar board pad 592 may include a distal end 591 and a proximal end 593 opposing distal end 591. Anchor 599 of spar board pad 592 may be disposed at proximal end 593.

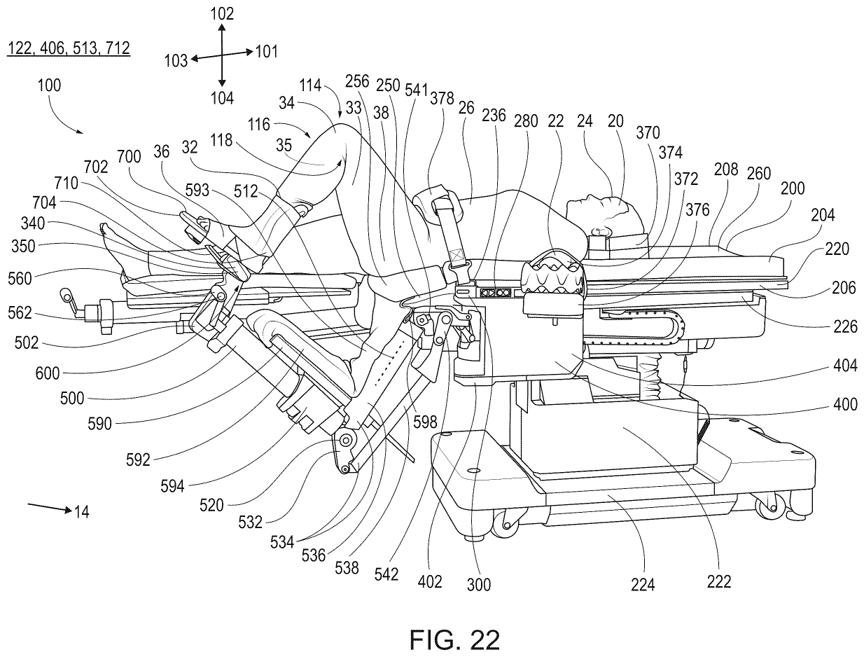

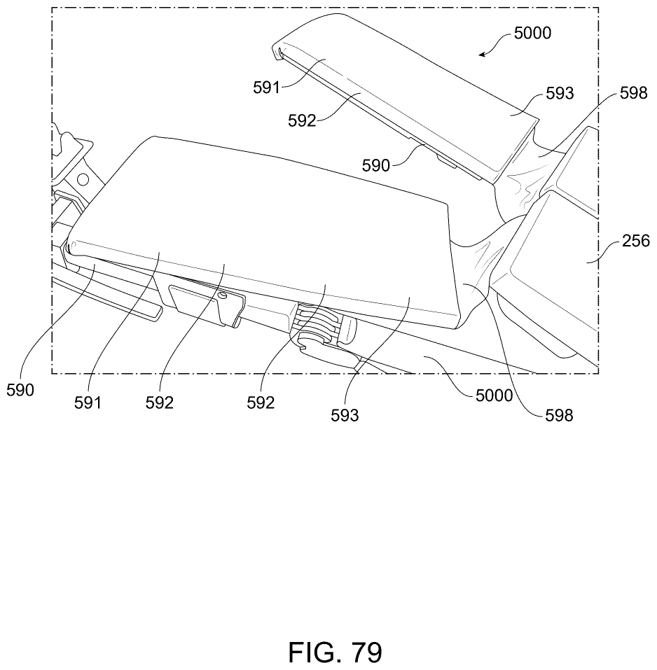

Proximal end 593 may also include a flexible portion 598 disposed between anchor 599 and spar board pad 592. Flexible portion 598 may be thinner than spar board pad 592. As spar 500 (FIG. 22) is flexed, flexible portion 598 may bundle between spar board pad 592 and knee ledge board pad 256 (FIG. 22) to which anchor 599 is coupled, as shown in FIG. 22. As spar 500 is extended, flexible portion 598 may unbundle from between spar board pad 592 and knee ledge board pad 256, providing additional material to allow translation of spar 500 in inferior direction 103 without detaching spar board pad 592 from knee ledge board pad 256. In addition, as shown in FIG. 79, as spar 5000 is yawed outward from centerline 110 (FIG. 1), flexible portion 598 may provide additional material to allow spar board pad 592 to yaw outward from centerline 110 along with spar 5000. As will be discussed spar 5000 may include any of the features of spar 500 and vice versa.

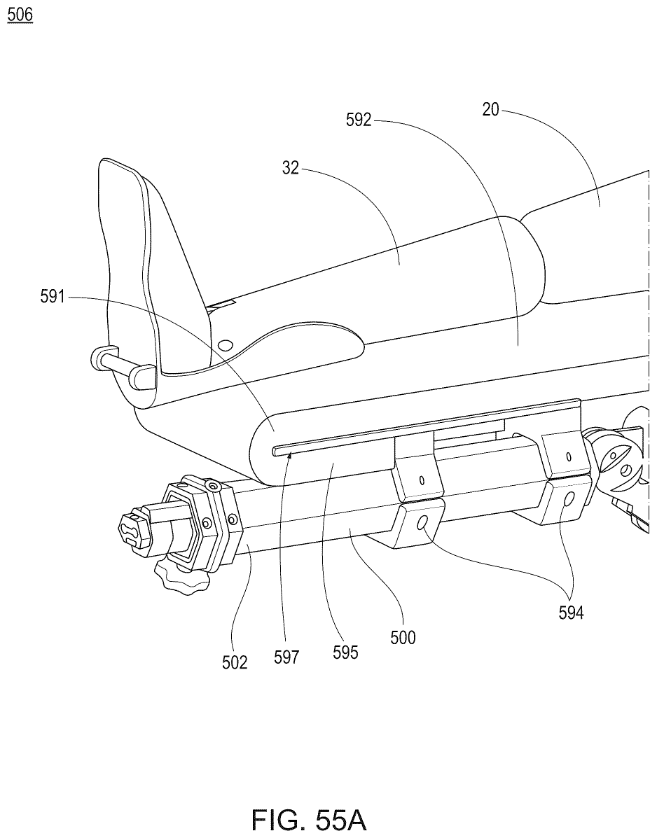

Distal end 591 may include a pad rollout 595. Pad rollout 595 may extend and retract relative to the remaining portion of spar board pad 592, and extend and retract relative to spar board 590, as shown with reference to FIGS. 55A-B according to exemplary embodiments. Pad rollout 595 may be tucked between spar board 590 and spar 500 and may secure around at least a portion of spar board 590 as spar board 590 is extended. A fastener 597 may secure pad rollout 595 to spar board 590. The fastener may be a hook and loop fastener, or may include another fastener such as a button, adhesive, a snap-fit joint, buckle, or other fastener.

In an example, spar board 590 may be disposed on distal end 502 of spar 500 shown in FIGS. 55A-B. Spar board pad 592 may be disposed on spar board 590. Excess of pad rollout 595 may be tucked between spar board 590 and spar 500. Proximal end 593 of spar board pad 592 may couple to knee ledge board pad 256.

Spar board pad 592 may be flexible to flex with spar 5000. As shown in FIG. 80, spar board pad 592 may fold as spar 5000 is flexed to be in folded spar configuration 513. Spar board pad 592 may include thinner foam and/or fewer foam layers than conventional pads to fold along with spar 5000. Also as shown in FIG. 80, as spar base 400 is in second position 408 between distal end 202 (FIG. 1) and proximal end 204 (FIG. 1) of surgery table 200 (FIG. 1) and spar 5000 is translated in superior direction 101 (FIG. 1) while flexed, flexible portion 598 may allow spar board pad 592 to remain coupled to spar 5000 and knee ledge board pad 256. Flexible portion 598 may provide additional material to extend spar board pad 592 such that spar 5000 may translate without spar board pad 592 detaching from spar 5000 and knee ledge board pad 256.

Referring again to FIGS. 52 and 54, in one embodiment, knee ledge board 250 and spar board pads 590 may be mounted on spar 500 (FIG. 1) in straight spar configuration 506 (FIG. 1). In this manner surgery table 200 (FIG. 1) may be used as a conventional surgery table with torso 26 (FIG. 20) and legs 32 (FIG. 20) of patient 20 (FIG. 20) supported by flat table surfaces. In this embodiment, spars 500 may be yawed with respect to one another, creating a gap between spars 500 in straight position 506. In this configuration, user 40 may stand in between spars 500 to have medial and lateral access to both legs 32 while both legs 32 are supported on spars 500 in straight spar configuration 506 and on flat surfaces of knee ledge board 250 and board pads 590. This is not possible on a conventional surgery table with only a platform on which the entire patient is supported.

In embodiments, any spar board 590 that is disposed on spar 500 (FIG. 1) may transition from supporting the leg 32 of patient 20 when spar 500 is in a straight configuration to “disappearing,” or no longer supporting the leg 32 of patient 20, such as shown in FIG. 55B when spar 500 flexes and/or translates such that the leg 32 of patient 20 bends, and spar board 590 flexes with spar 500 away from leg 32 of patient 20. In this manner, leg 32 may transition from supported by spar board 590, as shown in FIG. 55A, to being unsupported by spar board 590, as shown in FIG. 55B, and back again.

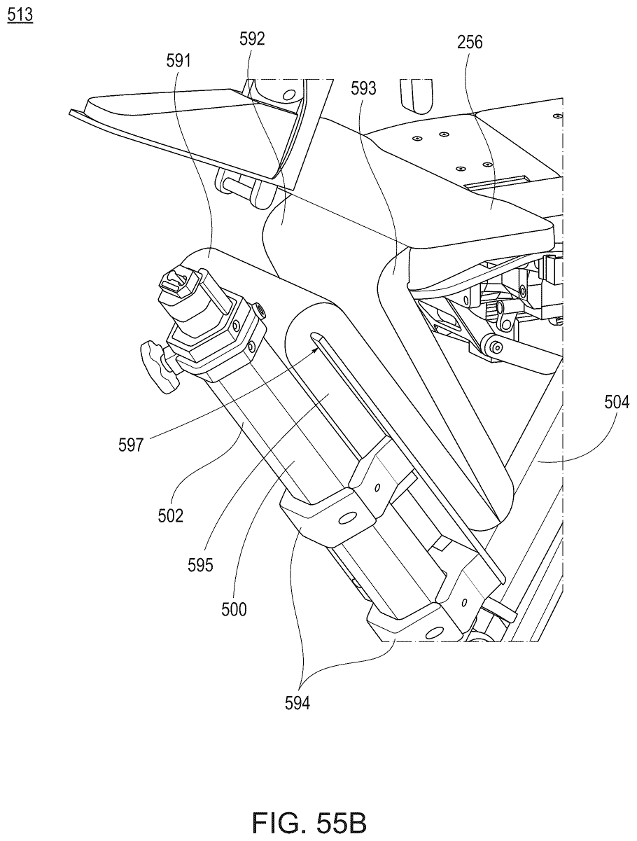

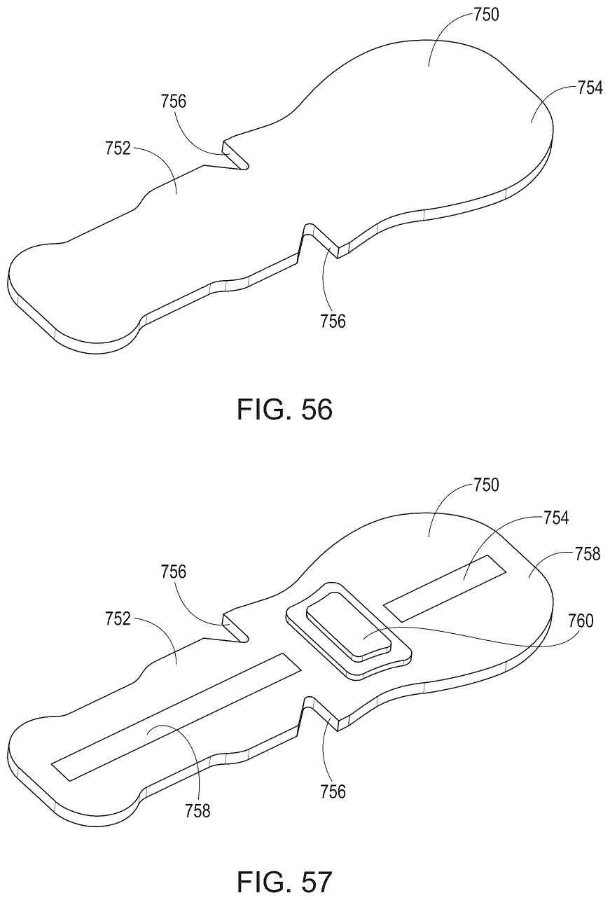

With reference to FIGS. 49-55, pads discussed herein, e.g., knee ledge board pad 256, trunk pad 260, and spar pads 592 may be disposable. Accordingly, pads may be single use and sterile. Pads discussed herein, e.g., knee ledge board pad 256, trunk pad 260, spar pads 592, and a foot support pad 750 and boards, e.g., knee ledge board 250 and spar board 590, may be part of positioning system 100. In another example, pads and boards may be a part of surgery table 200. In another example, pads and boards may function separately, such as with other positioning systems, surgery tables, or apparatuses thereof. In an example, pads and boards may include features that are functional separately and in combination. In another example, foot supports may receive pads. Foot support pad 750 is shown in FIGS. 56-57 according to exemplary embodiments.

Foot support pad 750 may be disposed on foot support 700 (FIG. 33), for example. Foot support pad 750 may provide a padded interface between foot 36 (FIG. 20) of patient 20 (FIG. 20) and foot support 700. Foot support pad 750 may include a flexible, cushioning material. Foot support pad 750 therefore may provide support and prevent pressure injuries. Foot support pad 750 may include a foot portion 752. Foot portion 752 may be disposed over foot portion 702 (FIG. 33) of foot support 700. Foot support pad 750 may also include a calf portion 754. Calf portion 754 may be disposed over calf portion 704 (FIG. 33) of foot support 700. Foot support pad 750 may secure to foot support 700 using adhesive 758. Adhesive 758 may be disposed on foot portion 752, calf portion 754, or foot portion 752 and calf portion 754. As shown, foot portion 752 and calf portion 754 may be coupled together. Foot support pad 750 may include a geometry to facilitate coupling with foot support 700 throughout positioning and supporting of patient 20 in a surgical procedure. Foot support pad 750 may include one or more cutouts 756 between foot portion 752 and calf portion 754. Accordingly, foot support pad 750 may fold when inserted in foot support 700. Foot support pad 750 may include a heel support 760. Heel support 760 may be proximate to an area where foot support pad 750 is folded to support a heel of foot 36 of patient 20. Heel support 760 may include thicker cushioning material and/or additional layers of cushioning material.

Other components of positioning system 100 may include covers and restraints. Covers discussed herein, e.g., a shoulder cover 550, an elbow cover 552, and an end cover 554, and restraints, e.g., an arm restraint 376, a torso restraint 378, and a leg restraint 382, may be part of positioning system 100. In another example, covers and restraints may be a part of surgery table 200. In another example, covers and restraints may function separately, such as with other positioning systems, surgery tables, or apparatuses thereof. In an example covers and restraints may include features that are functional separately and in combination.

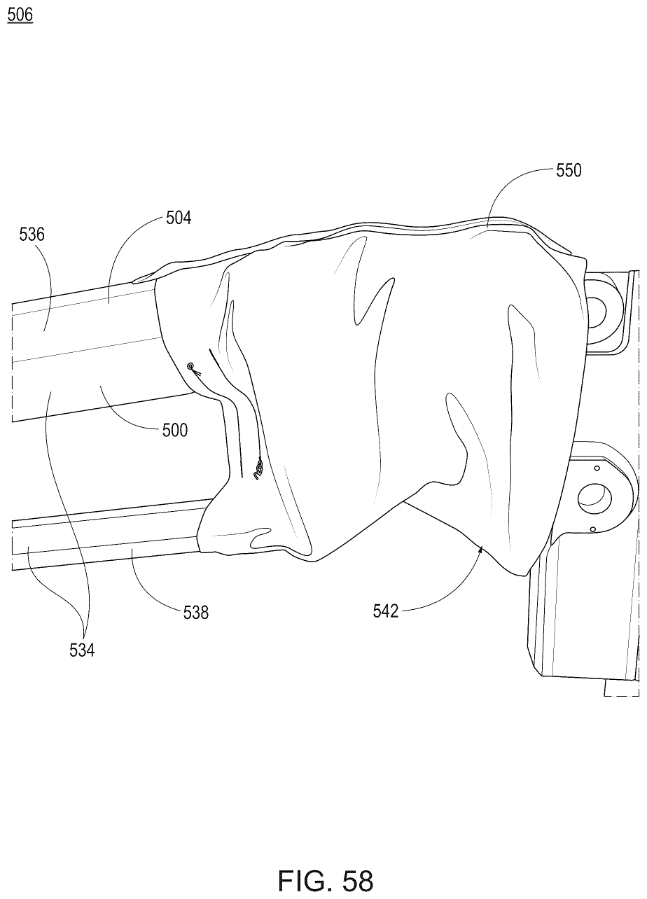

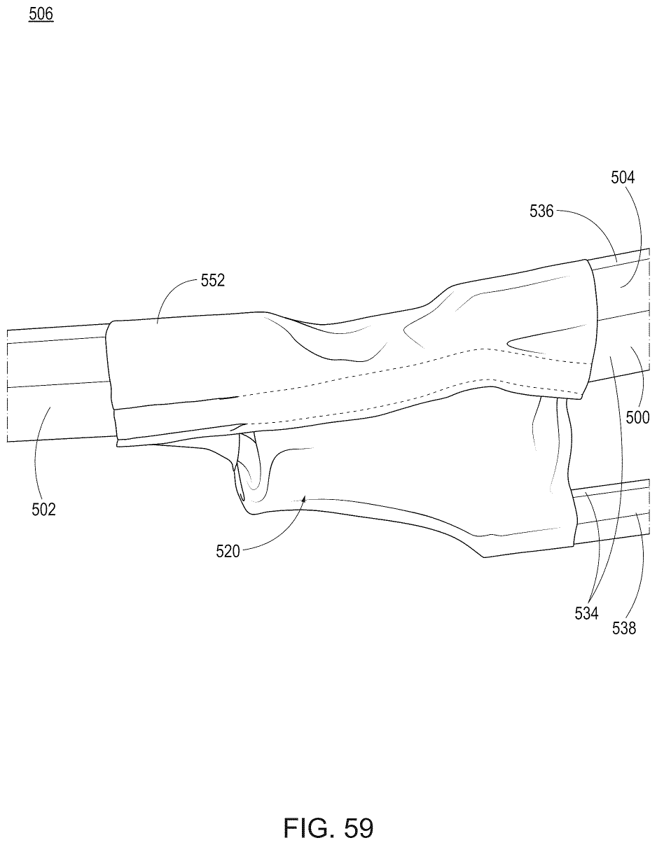

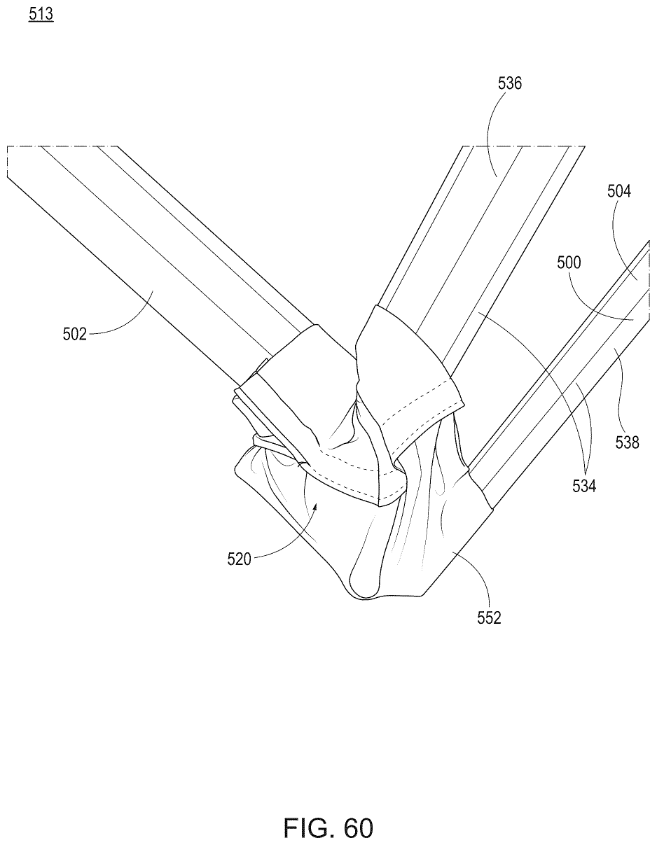

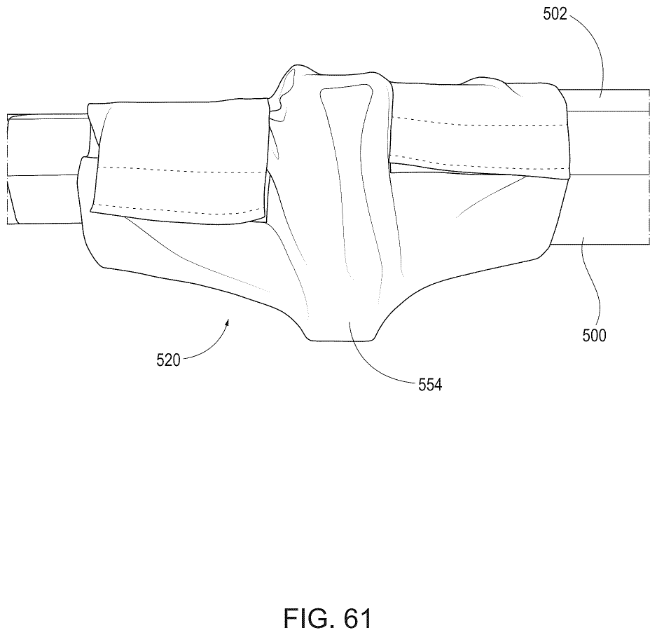

Covers may be disposable. Accordingly, covers may be single use and sterile. Covers may cover at least a portion of positioning system 100 (FIG. 1), such as a portion of surgery table 200 (FIG. 1), such as a portion of spar 500 (FIG. 3), or components thereof. For example, covers may be disposed over joints. In another example, covers may be disposed over subsystems of positioning system 100. In this way, covers maintain cleanliness of components during a surgical procedure and with repeated use. Example covers are shown in FIGS. 58-61 according to exemplary embodiments. FIG. 58 shows shoulder cover 550. Shoulder cover 550 may cover at least a portion of spar 500 (FIG. 3), such as a portion of proximal end 504 (FIG. 3) of spar 500. Shoulder cover 550 may cover at least a portion of spar base 400 (FIG. 1). In this way, shoulder cover 550 may cover at least a portion of one or more joints 420 (FIG. 14). Accordingly, shoulder cover 550 may cover at least a portion of spar 500 proximate to one or more joints 420. Shoulder cover 550 may thus protect one or more joints 420. FIG. 59-60 shows elbow cover 552. Elbow cover 552 may cover at least a portion of joint 520 (FIG. 3). FIG. 61 shows end cover 554. End cover 553 may cover at least a portion of distal end 502 (FIG. 3) of spar 500.

Referring to FIGS. 18-29, restraints may be used with patient 20. Restraints may be used to maintain patient 20 on surgery table 200. Restraints may be used in multiple surgical procedures such that they are reusable. Accordingly, restraints may be non-sterile. Restraints may maintain patient 20 on surgery table 200 and components thereof so that patient 20 does not fall off surgery table 200. Restraints may include hook and loop fasteners to be adapted for different sized patients.

As shown in FIG. 20, arm restraint 376 may be secured around at least a portion of arm 22 of patient 20. Arm 22 may be disposed on an arm board 372, which may extend laterally from surgery table 200, such as platform 220. An arm board pad 374 may be disposed on at least a portion of arm board 372. Accordingly, arm restraint 376 may be secured around at least a portion of arm 22, arm board 372, and arm board pad 374 to maintain arm 22 on surgery table 200. Arm board 372 may be substantially similar to or identical to arm boards described in U.S. Provisional Application No. 63/712,499 filed Oct. 27, 2024 titled “Arm Support for Surgical Positioning,” U.S. Provisional Application No. 63/769,459, and U.S. patent application Ser. No. 19/370,591 filed Oct. 27, 2025 titled “Arm Support for Surgical Positioning,” which are hereby incorporated by reference in their entireties.

Also as shown, torso restraint 378 may be secured around at least a portion of torso 26. Torso restraint 378 may couple to a handle 236 disposed on surgery table 200, such as lateral sides of surgery table 200, such as first side 206 and second side 208. Accordingly, torso restraint 378 may extend between handles 236 around at least a portion of torso 26 to maintain torso 26 on surgery table 200. Also as shown, leg restraint 382 may be secured around at least a portion of leg 32. Leg 32 may be disposed on spar board 590, which may be disposed on spar 500. Spar board pad 592 may be disposed on at least a portion of spar board 590. Accordingly, leg restraint 382 may be secured around at least a portion of leg 32, spar board 590, spar board pad 592, and spar 500 to maintain leg 32 on spar 500. Operable leg 32 may be flexed opposite to spar 500, as shown in FIG. 24. Thus, leg restraint 382 may be used to secure non-operable leg 32.

Various controls are shown with reference to FIGS. 41 and 62-64 according to exemplary embodiments. In an example, controls may be part of positioning system 100. In another example, controls may be a part of surgery table 200. In another example, controls may function separately, such as with other positioning systems, surgery tables, or apparatuses thereof. In an example, controls may include features that are functional separately and in combination.

As shown in the embodiment of FIG. 41 handle 560 may include one or more buttons, such as control 562, control 564, and control 565, to unlock various aspects of spar 500 (FIG. 3) to allow spar 500 to be articulated. Handle 560 may be disposed on distal end 502 (FIG. 3) of spar 500. Accordingly, handle 560 may be easily accessed by user 40 (FIG. 19). In one embodiment, the functions of handle 560, control 562, control 564, and control 565 may be dependent on which table module 340 (FIG. 1) is coupled to spar 500. For example, attaching foot support module 350 to UAI port 300 may allow for spar 500 articulation in pitch, yaw, and translation motions from handle 560, control 562, control 564, and control 565. Alternatively, spar 500 articulation in flex, pitch, yaw, and translation motions from handle 560, control 562, control 564, and control 565 may be allowed. In other words, positioning system 100 (FIG. 1), such as table 200 (FIG. 1), or components thereof, may communicate with table module 340. Depending on which table module 340 is attached, a computer system in positioning system 100, such as table 200, or components thereof, may change the function control 562, control 564, and control 565, to enable or disable certain motions including flex, pitch, yaw, and translation motions of spar 500.

In one embodiment, actuating control 565 may allow for spar 500 (FIG. 3) articulation in translation and yaw motions, or any motion that is not pitch or gravity-assisted. Actuating control 562 or control 564 may allow for spar 500 articulation in pitch motion. In one embodiment, control 562 or control 564 may be actuatable, or actuating control 562 or control 564 may acuate pitch, only when control 565 is actuated first. Accordingly, control 562 and control 564, or the pitch function of control 562 and control 564, may be locked until control 565 is actuated. In one embodiment, both control 562 and control 564 may allow for pitch motion of spar 500 to accommodate the handedness of user 40 (FIG. 19). In other embodiments, actuating control 565 and either control 562 and control 564 may allow for spar 500 to be flexed. Spar 500 may only be flexed by actuating control 565 and either control 562 or control 562 if foot support module 350 is coupled to spar 500.

Control 562, control 564, and control 565 may be toggled or momentarily actuated. In one embodiment, control 562 or control 564 may be pressed once to release pitch motion and pressed once again to lock pitch motion. Similarly, control 565 may be pressed once to release yaw and translation motions and pressed once again to lock yaw and translation motions. In another embodiment, holding control 562, control 564, and control 565 may allow the respective motion(s) to be engaged such that spar 500 is articulable in the respective motion(s) only when such button is being pressed. Releasing control 562, control 564, and control 565 thereafter may lock the respective motion(s).

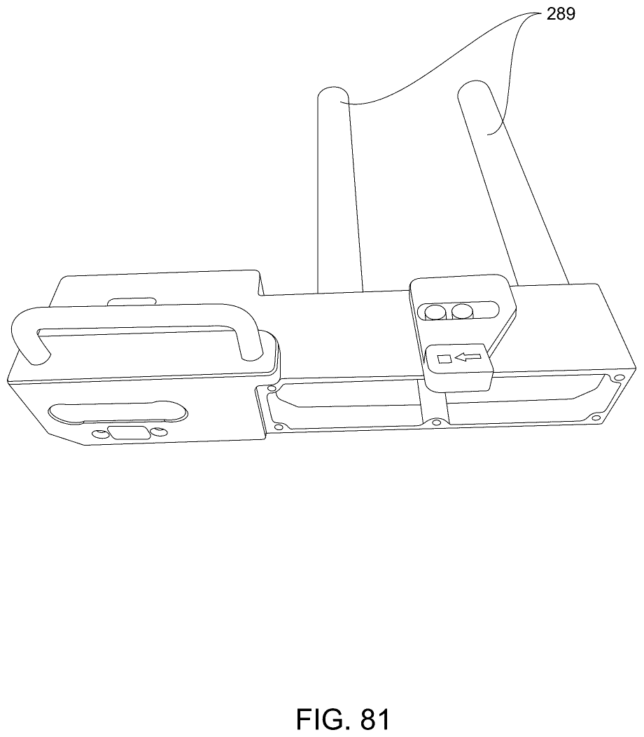

In one embodiment, one or more traction controls 280 shown in FIG. 62 may include one or more functionalities of handle 560 (FIG. 41). Traction controls 280 may be disposed along surgery table 200, such as along platform 220. Traction controls 280 may be extendable laterally from surgery table 200 with one or more traction control extensions 289 shown in FIG. 81 according to exemplary embodiments. Traction control extensions 289 may facilitate coupling traction controls 280 to surgery table 200. Traction control extensions 289 may retract into surgery table 200 or extend further outwardly from surgery table 200, sliding into or out of surgery table 200. In some embodiments, traction control extensions 289 may be locked into position to fix the lateral position of traction controls 280 with respect to surgery table 200. Traction controls 280 may be on the same module as a UAI port 300 (FIG. 41). Accordingly, retracting or extending traction controls 280 may retract or extend UAI port 300. UAI port 300 may be retracted or extended to accommodate different sized patients and receive table modules 340 (FIG. 1) accordingly.

Referring again to FIG. 62, traction controls 280 may include a first control 282. First control 282 may apply an indexed, or predetermined, translation of spar 500 (FIG. 3) to apply a traction load. In other words, pressing first control 282 once may translate spar 500 in one increment in inferior direction 103 (FIG. 1). Pressing first control 282 multiple times may translate spar 500 in corresponding multiple increments. Traction controls 280 may include a second control 284. In one embodiment, pressing second control 284 once may translate spar 500 in one increment in superior direction 101 (FIG. 1). Pressing second control 284 multiple times may translate spar 500 in corresponding multiple increments. Second control 284 may apply an indexed, or predetermined, translation of spar 500 to release at least a portion of the traction load.

Traction controls 280 may include a third control 286. One press of third control 286 may disengage traction mechanism 270 (FIG. 17), or electromechanical drive for spar base 400. In other words, translation of spar 500 from traction mechanism 270 to apply the traction load may be deactivated, e.g., lead screw 272 (FIG. 17) may be disengaged or neutralized. Accordingly, a traction load may then only be applied by user 40 (FIG. 19) manually moving spar 500 and corresponding spar base 400. Thus, third control 286 may allow for manual translation of spar 500. User 40 may manually position spar 500 before re-engaging traction mechanism 270 by another press of third control 286. Re-engaging traction mechanism 270 may hold the traction applied by user 40, and allows spar traction to be actuated once again by first control 282 and second control 284.

Traction controls 280 may include a fourth control 288. Fourth control 288 may allow for rotation of a foot support to support foot 36 (FIG. 20) of patient 20 (FIG. 20). A single press of fourth control 288 may unlock rotation and leg 32 (FIG. 20) of patient 20 may be rotated manually by user 40 (FIG. 19). Another press of fourth control 288 may lock rotation in the current orientation.

Traction control 286 and traction control 288 may be toggled or momentarily actuated. Accordingly, traction control 286 may be pressed once to disengage traction mechanism 270 (FIG. 17) and pressed once again to engage traction mechanism 270. Similarly, traction control 288 may be pressed once to allow for rotation of a foot support to support foot 36 (FIG. 20) of patient 20 (FIG. 20) and pressed once again to lock rotation. Holding traction control 286 may allow for traction mechanism 270 to be disengaged. Releasing traction control 286 thereafter may engage traction mechanism 270. Similarly, holding traction control 288 may allow for the foot support to be rotated. Releasing traction control 288 thereafter may lock rotation.

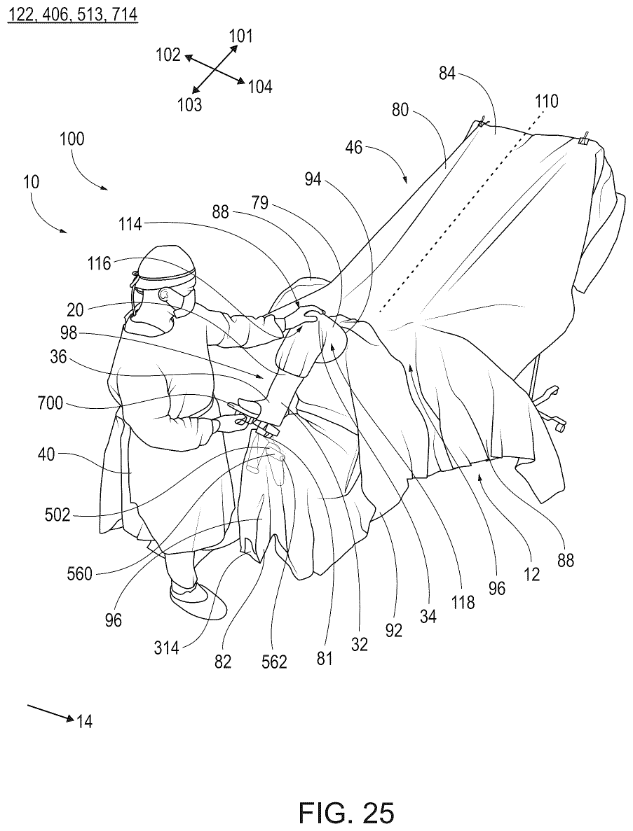

Pendant 290 shown in FIG. 63 may include one or more functionalities of handle 560 (FIG. 41), traction controls 280 (FIG. 62), or handle 560 and traction controls 280. Positioning system 100 (FIG. 1), such as surgery table 200 (FIG. 1), or components thereof, may include traction controls 291. For example, pendant 290 may include traction controls 291 to adjust a traction load and to engage or disengage traction. Pendant 290 may also include one or more controls 296 for surgery table 200, such as platform 220 (FIG. 1), to pitch and tilt the same. Pendant 290 may also include one or more controls 294 for articulating pelvic port adapter 242 (FIG. 47) and therefore attachments 240 (FIG. 48) coupled to pelvic port adapter 242. Pendant 290 may also include a display 297. Display 297 may provide user 40 (FIG. 19) with information about positioning system 100, such as surgery table 200, or components thereof. For example, display 297 may indicate that positioning system 100 is configured for knee surgery. Alternatively, display 297 may indicate that positioning system 100 is configured for another surgery, such as general surgery procedure, a specialized surgery procedure, or another orthopedic surgery, such as hip surgery. Table modules 340 (FIG. 1) may indicate which surgery is configured for. For example, foot support module 350 being coupled to spar 500 (FIG. 1) may indicate that knee surgery is configured for. Display 297 may also allow user 40 to reconfigure pedals 314 (FIG. 25). Pedals 314 may be foot switches from which user 40 may actuate different systems or apparatuses.

Pendant 290 may be functional proximate to positioning system 100 (FIG. 1), such as proximate to surgery table 200 (FIG. 1). User 40 (FIG. 19) may hold pendant 290 during at least a portion of a surgical procedure to use its controls. Positioning system 100 (FIG. 1), such as surgery table 200, spar 500 (FIG. 3), or surgery table 200 and spar 500 (FIG. 3) may receive pendant 290. For example, one or more drapes 46 (FIG. 18) may include one or more pockets 298 (not shown). Pockets 298 may receive pendant 290 to control at least articulation of spar 500. In this way, pendant 290 may be disposed in pocket 298 during at least a portion of a surgical procedure so that user 40 does not have to hold pendant 40 or find a location to store pendant 290. Pendant 290 may be disposed in pocket 298 without pocket 298 becoming contaminated from the outside.

Pocket 298 (not shown) may be disposable. Pocket 298 may include a pocket comprised partially or entirely of a film, or partially or entirely of a drape material, and may also include an adhesive, such as a pressure sensitive adhesive, on at least one side. Additionally or alternatively, pocket 298 may include another fastener, such as a button clasp, a magnetic attachment, or a hook and loop fastener. The fastener may facilitate attachment to one or more drapes 46 (FIG. 18). Pocket 298 may be coupled to one or more drapes 46 without damaging drapes 46 to maintain the sterile barrier. Pocket 298 may be selectively attachable. Pocket 298 may be selectively attachable to one or more drapes 46 for positioning system 100 (FIG. 1) along at least one of surgery table 200 (FIG. 1) and spar 500 (FIG. 1), such as along surgery table 200, along spar 500, or along surgery table 200 and spar 500. A plurality of pockets 298 may be disposed along surgery table 200, along spar 500, or along surgery table 200 and spar 500. Pocket 298 may include a sleeve to receive pendant 290 and a flap to fold over the sleeve and cover pendant 290. The flap may be secured to the sleeve with adhesive, such as a pressure sensitive adhesive, or another fastener, such as a button clasp, a magnetic attachment, or a hook and loop fastener.

Positioning system 100 (FIG. 1), such as surgery table 200 (FIG. 1) or spar 500 (FIG. 3), or components thereof, may include one or more manual controls. Manual controls may be actuated proximate to the component that is controlled. A manual lock 548 is shown in FIG. 64.

Spar 500 may be locked via manual lock 548. For example, joint 520 of spar 500 may be locked via manual lock 548. Additionally or alternatively, spar 500 articulation in pitch, yaw, and translation motions may be unlocked via manual lock 548. In other words, manual lock 548 may be used to alternately release and lock spar 500 to articulate spar 500 in at least one of flex, pitch, yaw, and translate motions. Manual lock 548 may be used in addition or alternatively to remote controls that are not proximate to joint 520 to lock and release spar 500.

Manual lock 548 may be proximate joint 520, such as directly below joint 520. Manual lock 548 may have a small profile. Accordingly, manual lock 548 may not affect imaging. At least a portion of manual lock 548 may be embedded in spar 500, such as joint 520. In other words, manual lock 548 may be formed in joint 520. In this way, manual lock 548 may include a small profile. Manual lock 548 may have a hook end that may be pulled by user 40 (FIG. 19) to assist in spar articulation, and in certain embodiments, translation for traction. For example, manual lock 548 may be pulled by user 40 (FIG. 19) to manually translate spar 500. Manual articulation of spar 500 may allow user 40 to feel spar 500 articulation, such as traction, and how patient 20 (FIG. 20) may respond to the articulation. In addition, manual articulation may allow for lower traction loads to be applied. Manual lock 548 may be actuated from sterile space 10 (FIG. 1). may Manual lock 548 may be located between the waist and chest of user 40 such that user 40 may actuate manual lock 548 from sterile space 10. Manual lock 548 may be actuated through any one or more drapes 46 (FIG. 18). Drapes corresponding to manual lock 548 may include a film portion so that the user may locate manual lock 548.

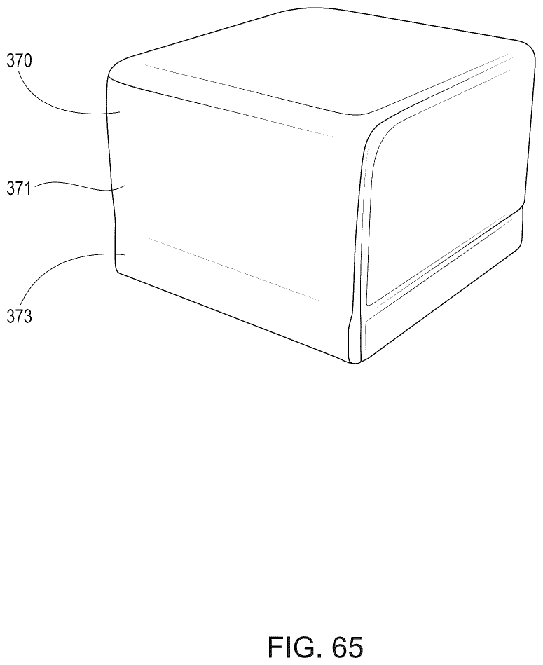

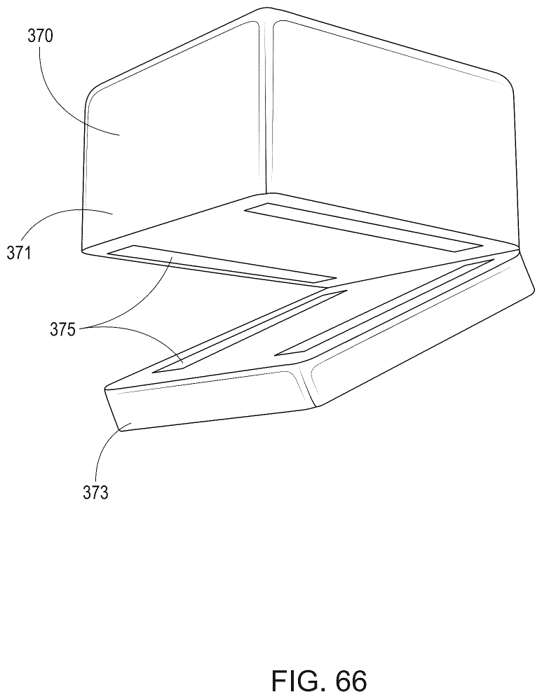

A head support 370 is shown in FIGS. 65-66 according to exemplary embodiments. Head support 370 may support patient 20 (FIG. 20), such as at least a head 24 (FIG. 20) of patient 20. Head support 370 may support patient 20 in a supine position. Additionally or alternatively, head support 370 may support patient 20 in a prone position. Additionally or alternatively, head support 370 may support patient 20 in a lateral position.

As shown, head support 370 may support patient 20 (FIG. 20) in a lateral position. Head support 370 may include a first foam 371 and a second foam 373. First foam 371 may be softer than second foam 373. First foam 371 may receive at least head 24 (FIG. 20) of patient 20. Arm 22 (FIG. 20) of patient 20 may extend laterally and may be disposed adjacent to head support 370. Second foam 373 may be pivoted outwardly to accommodate smaller sized patients. With reference to FIG. 66 first foam 371 and second foam 373 may include one or more fasteners, such as adhesive, buttons, or hook and loop fasteners, that may be alternately released and fastened to respectively outwardly pivot second foam 373 or couple first foam 371 and second foam 373. Head support 370 may receive a cover, which may be single use and sterile.

Positioning system 100 (FIG. 1), such as surgery table 200 (FIG. 1), or components thereof, may include lateral board 980, shown in FIG. 82 according to exemplary embodiments. Lateral board 980 may be coupled to one or more spars 500 (FIG. 1). For example lateral board 980 may be clamped to a part of spars 500. Lateral board 980 may be installed onto spar 500 laterally, e.g., lateral board 980 may be side-mounted onto spar 500 and fixed along spar 500. Lateral board 980 may be fixed to spar 500 via a clamp, for example, or a spar adapter also used for portable UAI 368 (not shown). Lateral board 980 may be static and not articulable. Lateral board 980 may extend from proximal end 504 (FIG. 1) to distal end 502 (FIG. 1) of spars 500 (FIG. 1). Alternatively, lateral board 980 may extend from proximal end 504 to a part of spars 500 between proximal end 504 and distal end 502. Lateral board 980 may support at least a portion of patient 20, such as when patient 20 is in a lateral position.

In knee surgery, lateral board 980 may support the non-operable leg. As lateral board 980 is mounted to spar 500, lateral board 980 may be articulated with spar 500, such as yawed or pitched. Lateral board 980 may be used on either spar 500 to support either leg. Lateral board 980 may include a width of about two-thirds of surgery table 200 (FIG. 1) to balance support for patient 20 (FIG. 18) and surgical access. As shown, lateral board 980 may include a cutout 982. Cutout 982 may create a concavity along a side of lateral board 980 such that lateral board 980 has less material but still may support patient 20 in a lateral position. In addition, cutout 982 may support user 40 (FIG. 19) in standing between spars 500 such as to access operable leg 32 (FIG. 20) directly from medial and lateral sides. Edges of lateral board 980 may be engineered to minimize risk of pressure injury. Lateral board 980 may be radiolucent. Lateral board 980 may include carbon fiber.

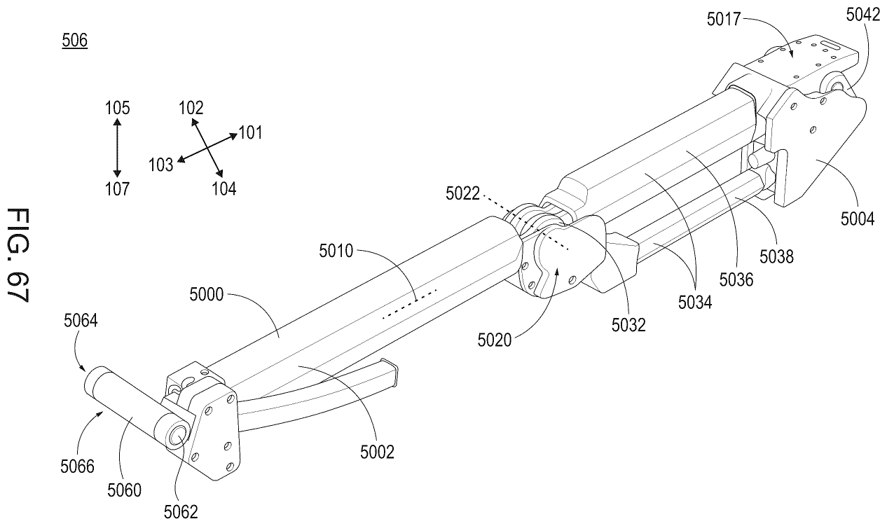

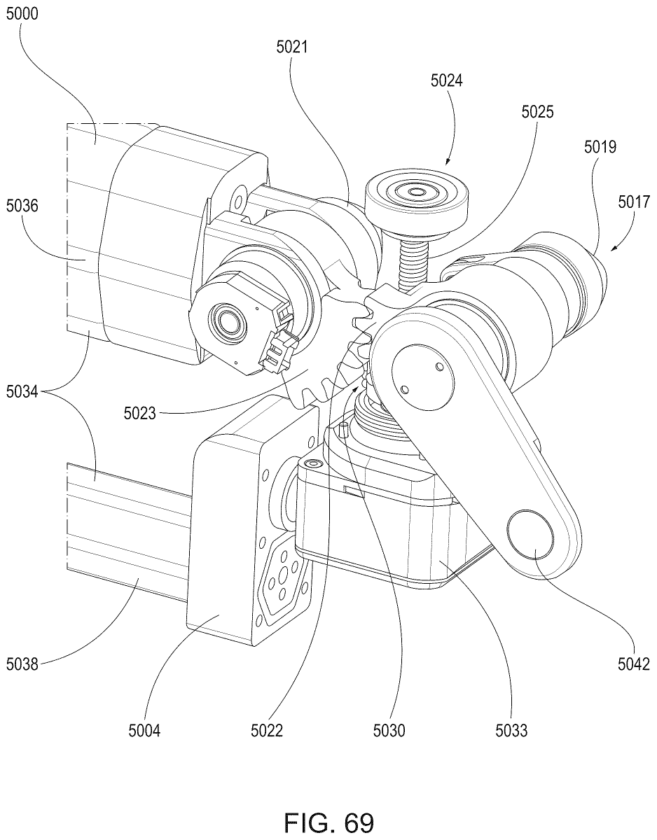

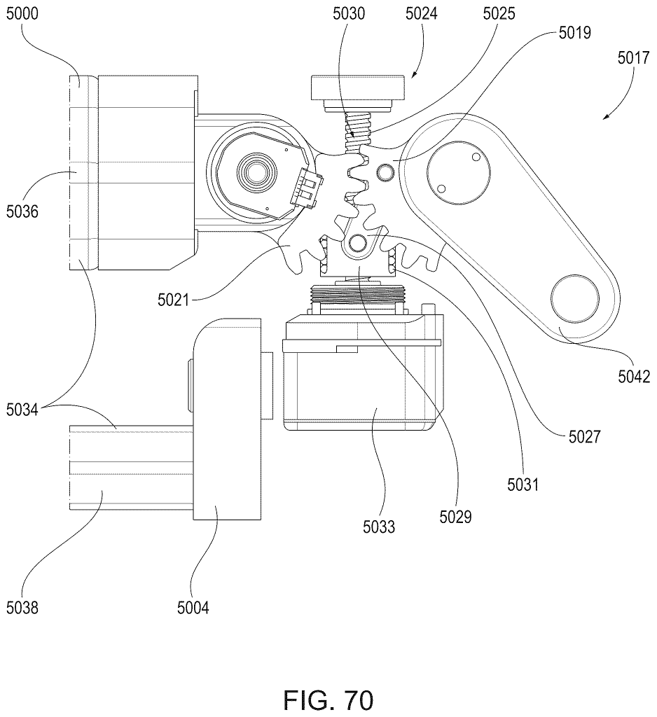

Spar 5000 is shown with reference to FIGS. 67-73 according to exemplary embodiments. Spar 5000 may include any of the features of spar 500 (FIG. 3) discussed above and vice versa. Accordingly, in an example, spar 5000 may be part of positioning system 100 (FIG. 1). In another example, spar 5000 may be a part of surgery table 200 (FIG. 1). In another example, spar 5000 may function separately, such as with other positioning systems, surgery tables, or apparatuses thereof. In an example, spar 5000 may include features that are functional separately and in combination.

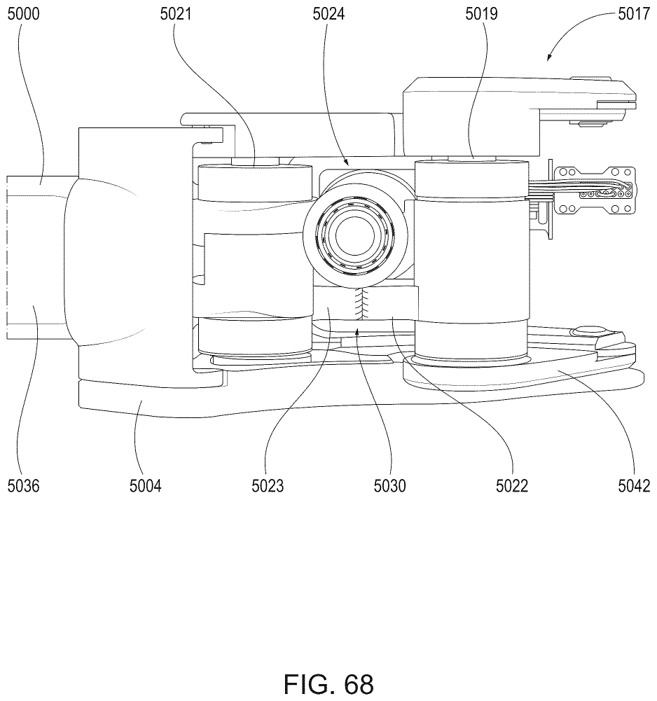

As spar 5000 flexes, proximal end 5004 may fold toward distal end 5002. Referring to FIGS. 68-70, in certain embodiments, proximal end 5004 may include one or more gears 5030 that may control the orientation of proximal end 5004. For example, one or more gears 5030 may include two gears 5030, such as a proximal gear 5022 and a distal gear 5023. Gears 5030 may be arranged as a pair. Gears 5030 may counterrotate relative to one another and mesh together. Proximal gear 5022 of the pair may be coupled to a proximal rotational shaft 5019. Distal gear 5023 of the pair may be coupled to a distal rotational shaft 5021, which may be coupled to first link 5036 to control the orientation of first link 5036 of spar 5000. With reference to the discussion above for spar 500 (FIG. 3), spar 5000 may include passive link 532 (FIG. 5), stationary link 541 (FIG. 5), and rotating link 542 (FIG. 5). Gears 5030 counterrotating and meshing may cause rotating link 542 to rotate at substantially the same time as gears 5030 rotate. Rotating link 542 may rotate towards stationary link 541, as shown in FIG. 5. Accordingly, link 5036 and link 5038 may be moved such that link 5036 and link 5038 remain parallel to one another. Passive link 532 may rotate in inferior direction 103 (FIG. 1) to compensate for rotating link 542 rotating toward stationary link 541. Rotating link 542 may be rotated away from stationary link 541 and passive link 532 may rotate in superior direction 105 (FIG. 1) as spar 5000 is unfolded at least partially, such as unfolded fully, to reach straight spar configuration 506. Accordingly, in embodiments, the pitch of distal end 5002 may be controlled by rotation of passive link 532, which is linked via gears 5030 to rotation of proximal end 5004.

Referring again to FIGS. 68-70, in other embodiments, a locking mechanism 5017 of spar 5000 may lock the orientation of proximal end 5004. Accordingly, the orientation of distal end 5002 may be locked. Proximal end 5004 may include locking mechanism 5017. Locking mechanism 5017 may lock first link 5036. Accordingly, proximal end 5004 may be prevented from folding toward distal end 5002. Locking mechanism 5017 may including proximal rotational shaft 5019, distal rotational shaft 5021 coupled to first link 5036, and gears 5030. Locking mechanism 5017 may include gears 5030 to alternately rotate and lock first link 5036. As shown, proximal gear 5022 coupled to proximal rotational shaft 5019 may be coupled to distal gear 5023 coupled to distal rotational shaft 5021 such that distal rotational shaft 5021 may rotate relative to proximal rotational shaft 5019 as gears 5030 rotate relative to one another. Locking proximal rotational shaft 5019 may lock distal rotational shaft 5021 to lock first link 5036 in position as gears 5030 stop rotating relative to one another when proximal rotational shaft 5019 is stopped from rotating.

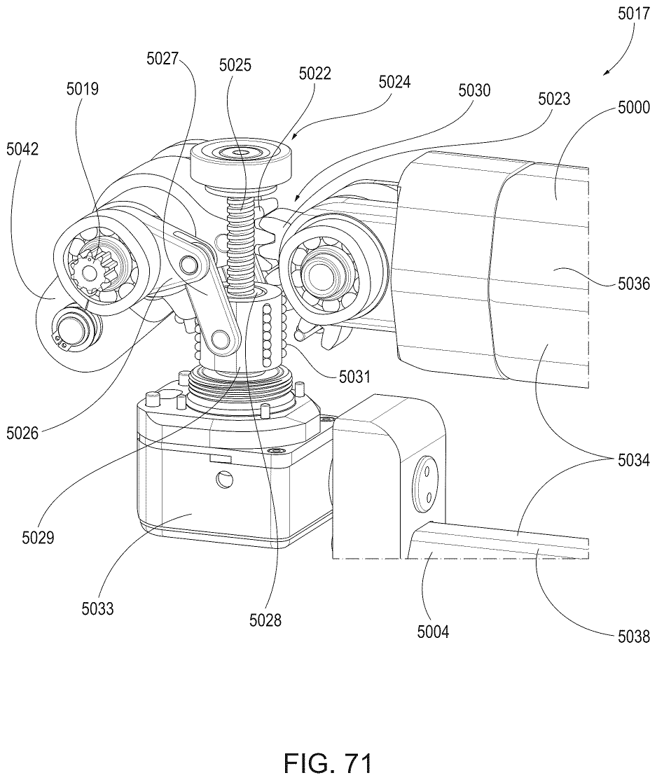

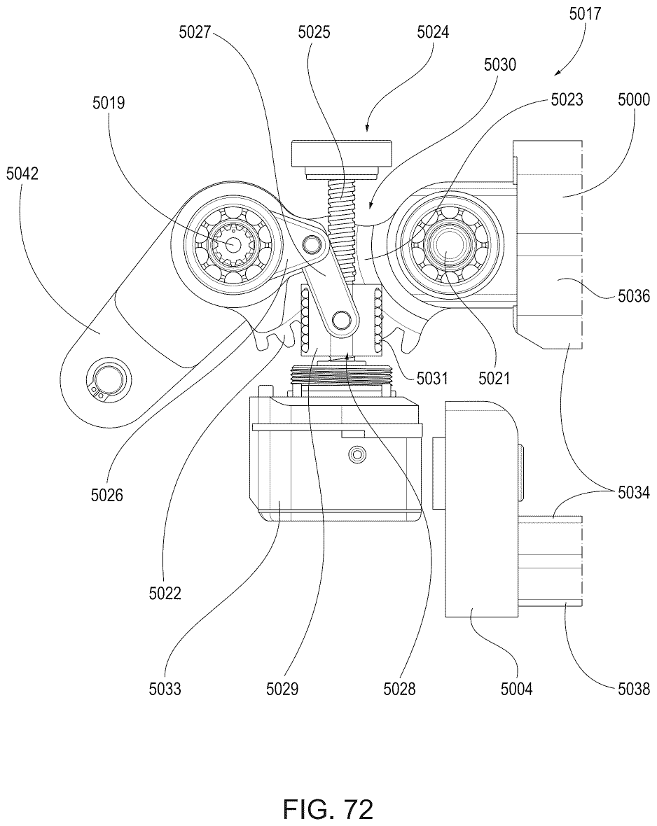

Referring to the embodiments shown in FIGS. 71-72, proximal rotational shaft 5019 may be rotated or stopped from rotating by a slider-crank mechanism 5024. Accordingly, slider-crank mechanism 5024 may control the orientation of proximal end 5004, and locking mechanism 5017 may include slider-crank mechanism 5024 to lock proximal rotational shaft 5019 and therefore first link 5036. In one embodiment, slider-crank mechanism 5024 may include an electromagnetic brake 5033. When electromagnetic brake 5033 is unlocked, proximal rotational shaft 5019 may be rotated. When electromagnetic brake 5033 is locked, proximal rotational shaft 5019 may be stopped from rotating. Slider-crank mechanism 5024 may also include a ball screw 5025 and a ball nut 5028 coupled to ball screw 5025. Ball screw 5025 may rotate and ball nut 5028 may translate vertically along ball screw 5025 when electromagnetic brake 5033 is unlocked. Ball screw 5025 may stop rotating and ball nut 5028 may be locked in position when electromagnetic brake 5033 is locked.

In embodiments, slider-crank mechanism 5024 may also include a slider link 5027. Slider link 5027 may be coupled to proximal rotational shaft 5019. Slider link 5027 may be coupled to a crank arm 5026, which is coupled to proximal rotational shaft 5019. Accordingly, slider link 5024 may rotate proximal rotational shaft 5019 when electromagnetic brake 5033 is unlocked. Slider link 5027 may be locked in position when electromagnetic brake 5033 is locked such that proximal rotational shaft 5019 is locked to lock first link 5036.

As shown in one embodiment, slider link 5027 may be coupled to ball nut 5028 and proximal rotational shaft 5019. Electromagnetic brake 5033 may be coupled to ball screw 5025 to alternately rotate and stop rotation of ball screw 5025 and alternately vertically translate and stop vertical translation of ball nut 5028. Ball nut 5028 may vertically translate along with a slider housing 5029. Ball nut 5028 may be housed by slider housing 5029 having slider bearings 5031 to rotate around ball screw 5025. Slider housing 5029 having ball nut 5028 may vertically translate along ball screw 5025, thereby vertically translating slider link 5027 to rotate proximal rotational shaft 5019 to adjust the orientation of first link 5036. In other words, ball screw 5025 may rotate such that ball nut 5028 translates vertically when electromagnetic brake 5033 is unlocked, causing slider link 5027 to translate vertically and rotate proximal rotational shaft 5019 to adjust the orientation of first link 5036. Ball screw 5025 may stop rotating when electromagnetic brake 5033 is locked such that slider link 5027 is locked in position to lock proximal rotational shaft 5019 to lock first link 5036. First link 5036 may be locked as it is coupled to distal rotational shaft 5021, which may be geared to proximal rotational shaft 5019.

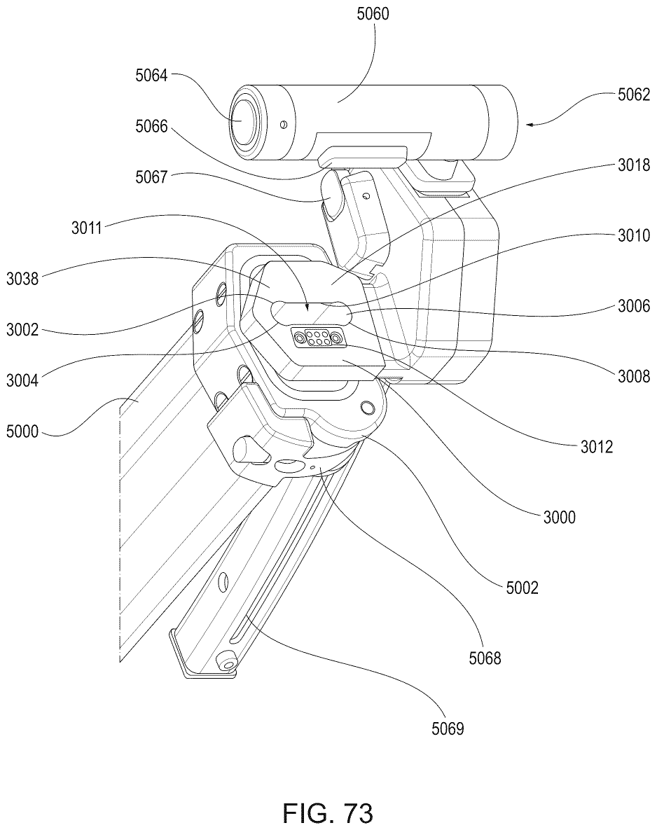

One embodiment of distal end 5002 of spar 5000 is shown in FIG. 73. As discussed above, distal end 5002 of spar 5000 may include handle 5060 to allow user 40 (FIG. 19) to articulate spar 5000. Distal end 5002 may also include one or more joint controls. In embodiments, center joint 5020 (FIG. 67) may be locked in a particular orientation when a corresponding user interface is released. Accordingly, spar 5000 may be stiff, or generally immovable, when center joint 5020 is locked. Handle 5060 may include one or more joint controls, such as button 5062, button 5064, and a button 5066. At least one of button 5062, button 5064, and button 5066 may include joint controls. Button 5062 may be similar to control 562 (FIG. 5) discussed above. Control 564 may be similar to control 564 (FIG. 5) discussed above. Button 5066 may be a pressure plate. Button 5066 may be similar to control 565 (FIG. 5) discussed above. In one embodiment, actuating button 5066 may allow for spar 5000 (FIG. 67) articulation in translation and yaw motions, similar to control 565.

Handle 5060 may include a handle extension lock 5067. Handle extension lock 5067 may have a safety function. Accordingly, handle extension lock 5067 may be released to allow handle 5060 to be repositionable. As shown, a handle extension slider 5069 may be extended relative to spar 5000 to extend handle 5060 in inferior direction 103 (FIG. 1). Alternatively, handle extension slider 5069 may be retracted relative to spar 5000 to retract handle 5060 in superior direction 101 (FIG. 1). Handle 5060 may include a spar extension lock 5068, which may also have a safety function. Accordingly, spar extension lock 5068 may be released to allow spar 5000 to be lengthened or shortened. Distal end 5002 may include a telescoping means to allow spar 5000 to lengthen or shorten. Proximal end 5004 (FIG. 67) may be positioned at a generally constant longitudinal distance relative to platform 220 (FIG. 1) as distal end 5002 is repositioned longitudinally relative to platform 220 by releasing spar extension lock 5068.

Referring to FIGS. 3 and 67, Spar 500 and spar 5000 may articulate relative to surgical table 200 (FIG. 1). Spar 500 and spar 5000 may be positioned relative to centerline 110 (FIG. 1) of positioning system 100 (FIG. 1), or surgical table 200, to facilitate articulation. Spar 500 and spar 5000 may be positioned laterally at a farther distance from centerline 110 to facilitate a wider range of articulation. Articulation may include pitch and yaw motions. Articulation may be described relative to straight spar configuration 506, in which spar 500 and spar 5000 extend along a horizontal plane of surgical table 100 and along axes generally parallel to centerline 110. Accordingly, in straight spar configuration 506, spar 500 and spar 5000 are not pitched or yawed. Spar 500 and spar 5000 may pitch in an upward direction from straight spar configuration 506 between about five degrees and about 40 degrees, such as between about 10 degrees and about 30 degrees, such as about 20 degrees. Spar 500 and spar 5000 may pitch in a downward direction from straight spar configuration 506 between about five degrees and about 50 degrees, such as between about 20 degrees and about 40 degrees, such as about 36 degrees. Spar 500 and spar 5000 may yaw from straight spar configuration 506 inward toward centerline 110 between about five degrees and about 40 degrees, such as between about 10 degrees and about 30 degrees, such as about 20 degrees. Spar 500 and spar 5000 may yaw from straight spar configuration 506 outward away from centerline 110 between about five degrees and about 60 degrees, such as between about 20 degrees and about 50 degrees, such as about 40 degrees.

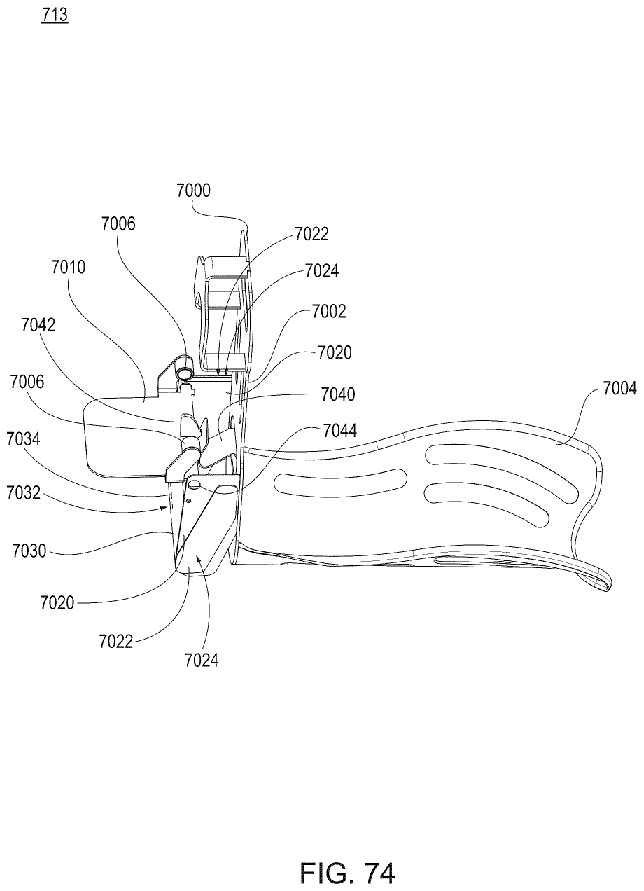

Foot support 7000 is shown in FIGS. 74-77 according to exemplary embodiments. Foot support 7000 may include any of the features of foot support 700 (FIG. 33) discussed above and vice versa. Accordingly, in an example, foot support 7000 may be part of positioning system 100 (FIG. 1). In another example, foot support 7000 may be a part of surgery table 200 (FIG. 1). In another example, foot support 7000 may function separately, such as with other positioning systems, surgery tables, or apparatuses thereof. In an example, foot support 7000 may include features that are functional separately and in combination.

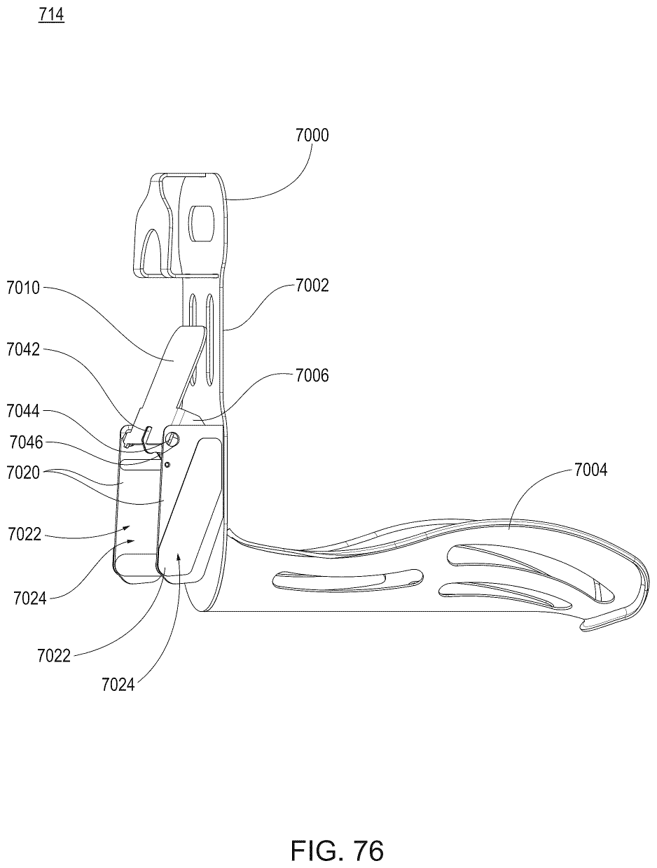

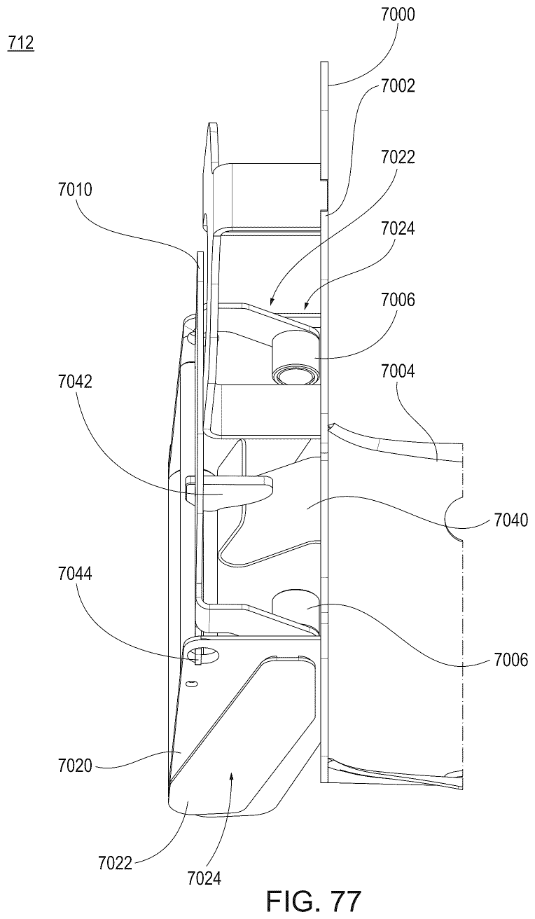

Foot support 7000 in an open position 713 is shown in FIG. 74. As discussed above for foot support 700 (FIG. 33), foot support 7000 may include user interface paddle 7010 having extended position 712 (FIG. 77). Extended position 712 may be outward of retracted position 714 (FIG. 76). User interface paddle 710 may be pivoted outward of extended position 712, as shown in FIG. 74. In this way, a wrapping may secure around foot 36 (FIG. 20) and foot portion 7002 of foot support 7000 to secure foot 36 to foot support 7000. Once the wrapping is complete, user interface paddle 7010 may be rotated back into extended position 712. Accordingly, open position 713 may be outward of extended position 712.

User interface paddle 7010 may release foot support 7000 from a foot support module, such as foot support module 350 (FIG. 39). Accordingly foot support 7000 may be rotatable relative to foot support module 350. In certain positions, foot support 7000 may be locked relative to foot support module 350. Accordingly, foot support 7000 may not be rotatable. In such positions, user interface paddle 7010 may be locked in position as well. Accordingly, user interface paddle 7010 may not be rotatable relative to the rest of foot support 7000 and foot support module 350.

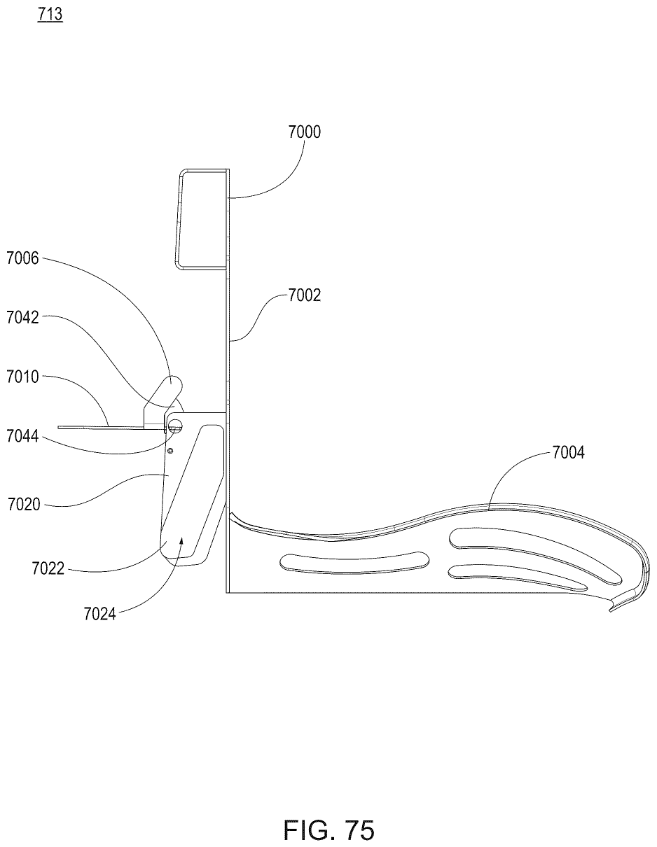

With reference to FIGS. 74-75, user interface paddle 7010 may be locked in position as foot support 7000 is in open position 713. With reference to FIG. 76, user interface paddle 7010 may not be locked in position as foot support 7000 is in retracted position 714. With reference to FIG. 77, user interface paddle 7010 may be locked in position as foot support 7000 is in extended position 712. Accordingly, user interface paddle 7010 may include a first position and a third position, extended position 712 and open position 713, and user interface paddle 7010 may be locked in at least one of the first position and the third position. User interface paddle 7010 may include a second position, retracted position 714, in which user interface paddle 7010 is not locked.

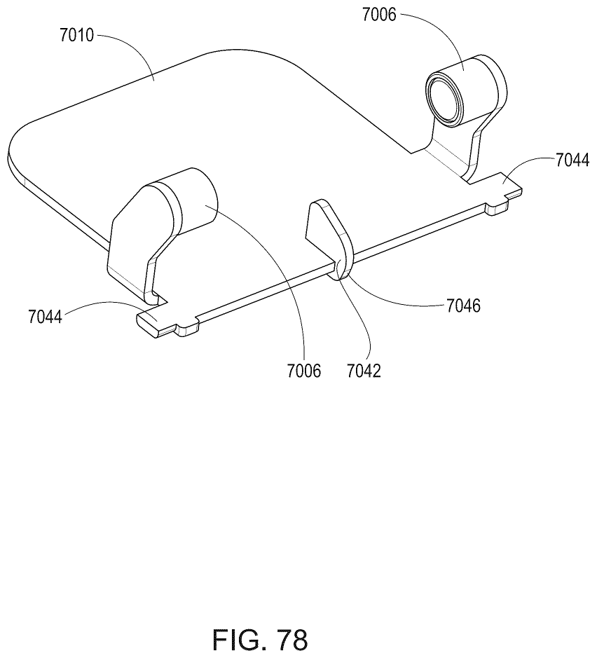

As best shown in FIG. 78 according to an exemplary embodiment, user interface paddle 7010 may include a detent 7042. Detent 7042 may lock user interface paddle 7010 in one or more positions. For example, detent 7042 may lock user interface paddle 7010 in extended position 712. Additionally or alternatively, detent 7042 may lock user interface paddle 7010 in open position 713.

Referring to FIGS. 74-78, user interface paddle 7010 may be actuated by being rotated inward. Rotation inward may be toward foot portion 7002 of foot support 7000. Rotation inward may be against one or more springs 7040. As shown, foot support 700 may include one or more springs 7040. Foot support 7000 may be biased to extended position 712. Additionally or alternatively, foot support 7000 may be biased to open position 713. Accordingly, rotation of user interface paddle 7010 to retracted position 714 may be against the bias of one or more springs 7040.

As discussed, detent 7042 may lock user interface paddle 7010 in one or more positions. As best shown in FIG. 78, detent 7042 may include one or more flat portions 7046. One or more flat portions 7046 may receive one or more springs 7040 such that pressure from one or more springs 7040 secures user interface paddle 7010. In this way, user interface paddle 7010 may be locked in position.

As best shown in FIG. 78, user interface paddle 7010 may include one or more flat heads 7044. Flat heads 7044 may rotate in corresponding openings 7046 in foot support 7000 (FIG. 74) to rotate user interface paddle 7010 relative to foot support 7000. Flat heads 7044 7044 may include a generally flat geometry. Accordingly, flat heads 7044 may not fill corresponding openings 7046 in comparison to round pins, which may fill openings 7046 and continuously contact the sidewalls of openings 7046. With fewer points of contact between flat heads 7044 and the sidewalls of corresponding openings 7046, flat heads 7044 and the sidewalls of corresponding openings 7046 may be more easily cleaned and sterilized.

Foot support module may include a cutoff switch (not shown), or a quick release mechanism to deenergize magnetic sensor 354 (FIG. 37). This may allow foot support module 350 (FIG. 39) to release foot support 7000 in any position.

Non-operable leg 32 may be supported by one or more components. As discussed herein, spar 500 (FIG. 3) may support non-operable leg 32 by securing foot 36 (FIG. 20) of patient 20 (FIG. 20) to a foot support coupled to spar 500. Additionally or alternatively, non-operable leg 32 may be supported on lateral board 980 (FIG. 82) or imaging board 984 (FIG. 83). Non-operable leg 32 may be positioned before the surgical procedure commences. Accordingly, non-operable leg 32 may be pre-operatively placed and then draped. Selection of which support is to be used for non-operable leg 32 may be based on procedure length, patient comfort, and user 40 (FIG. 19) preference.

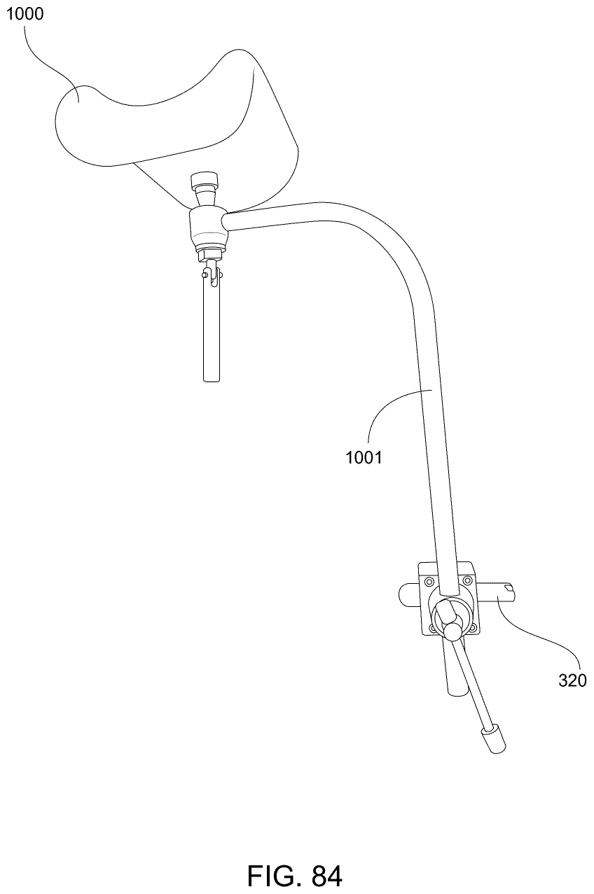

As shown in FIG. 84 according to exemplary embodiments, non-operable leg 32 may be supported by a leg holder 1000. Non-operable leg 32 may rest on leg support 1000 and may hang off of leg holder 1000. As shown, leg holder 1000 may include a cradle-shape with upwardly sloping sidewalls to support non-operable leg 32, and downwardly sloping edges to allow non-operable leg 32 to hang off of well leg support 1000. Leg holder 1000 may couple to spar 500 (FIG. 3) via a UAI plug 320. UAI plug 320 of leg holder 1000 may be coupled to UAI port 300 (FIG. 41) at distal end 502 (FIG. 3) of spar 500. Alternatively, UAI plug 300 of leg holder 1000 may be coupled to portable UAI 368 having UAI port 300. UAI plug 320 may be separated from leg holder 1000 by an arm 1001. In this way, leg holder 1000 may position non-operable leg 32 away from operable leg to facilitate the surgical procedure. Leg holder 1000 may include a smaller support surface, offering flexibility for shorter procedures, such as medium-length procedures.

Other supports for non-operable leg 32 may include a foot sling (not shown). The foot sling may be similar to a “candy cane” stirrup known in the art. In positioning system 100 (FIG. 1), the foot sling may be devoid of the “candy cane” structure and, instead, an arm can attach the sling to spar base 400 and hold the sling below distal end 502 (FIG. 3) of spar 500. The foot sling may couple to spar 500 at distal end 502, such as by coupling to UAI port 300 (FIG. 41). Because of the lightweight and flexible structure of the foot sling, the foot sling may be less stable than leg holder 1000 but also may be patient-friendly by applying less pressure to patient 20 (FIG. 20) by allowing leg 32 to hang freely. The foot sling may be desirable for shorter procedures than leg holder 1000. Another support may be a leg stirrup (not shown) known in the art, which may be desirable for longer procedures. The leg stirrup may provide stability and support for longer procedures but may be bulkier. The leg stirrup, like leg holder 1000 and the foot sling, may be coupled to spar 500 at distal end 502, such as by coupling to UAI port 300. Each well leg support may articulate with spar 500. Spar 500 articulation is described herein.

With reference to FIGS. 1-84 and the discussion herein, a surgical method may include supporting at least a portion of leg 32 of patient 20 on foot support 700 at distal end 502 of spar 500 extending from surgery table 200, placing drape 46 (e.g., top drape 80) over foot support module 350 coupled to spar 500 so that sterile space 10 is above drape 46 and working space 12 is below, coupling foot support 700 to foot support module 350 with drape 46 in between so that foot support 700 is in sterile space 10 and foot support module 350 is in working space 12, generating a magnetic field in sterile space 10 by magnets 706 in foot support 700 by moving magnets 706 proximal to a mu-metal pathway such as sidewall mu-metal conduit 722 within foot support 700 to extend the magnetic field through conduit 722 to a magnetic field terminus such as rod 730 including mu metal 732, positioning the magnetic field terminus proximate magnetic sensor 354 in foot support module 350 so that the magnetic field extends across drape 46 from sterile space 10 to working space 12, and actuating magnetic sensor 354 in working space 12 via the magnetic field extending from rod 730 to magnetic sensor 354.

In some embodiments, foot support 700 may further include a foot support attachment that provides the magnetic field terminus, which may be foot support 700, such that the magnetic field may actuate foot support module 350 to grip the foot support attachment via gripping mechanism 352 across drape 46. The method may include moving a user interface of foot support 700, such as user interface paddle 710, between a first position in which magnets 706 are not proximal to mu-metal pathway 722 and do not generate a magnetic field at the magnetic field terminus, and a second position in which magnets 706 are proximal to mu-metal pathway 722 and extend the magnetic field to the magnetic field terminus; user interface paddle 710 may be biased to the first position by springs 740, and may include a detent configured to lock the user interface in at least one of the first position or a third position outward of the second position. In the second position of user interface paddle 710, distal end 502 of spar 500 may fold and proximal end 504 of spar 500 may translate substantially simultaneously. The magnetic field terminus may include rotational axis 734 about which foot support 700 may rotate in a pitch direction relative to foot support module 350. The method may further include releasing joint 520 disposed between and coupling proximal end 504 and distal end 502 of spar 500, and flexing spar 500 generally in a posterior direction by releasing joint 520 such that an inferior window 116 is formed proximate spar 500 to allow access to a knee generally from an inferior direction. Magnets 706 may actuate mechanical coupling 600 configured to couple foot support 700 to foot support module 350. Foot support module 350 may include a switch configured to de-energize magnetic sensor 354.

A system may include a surgery table 200 having distal end 202 and proximal end 204 and platform 220 configured to support at least a portion of a patient 20; spar base 400 coupled to surgery table 200 and positioned beneath platform 220; spar 500 with proximal end 504 coupled to and extending from spar base 400 and distal end 502 to support a leg; foot support 700 coupled at distal end 502 of spar 500 and comprising a mu-metal pathway such as sidewall conduit 722 and a magnetic field terminus comprising mu metal such as rod 730 with mu metal 732 opposite magnets 706; foot support module 350 coupled to distal end 502 and comprising magnetic sensor 354; and drape 46 (e.g., top drape 80) configured to be placed over foot support module 350 to define sterile space 10 above and working space 12 below, with foot support 700 configured to couple to foot support module 350 with drape 46 in between so that foot support 700 is in sterile space 10 and foot support module 350 is in working space 12; wherein magnets 706 are configured to generate a magnetic field in sterile space 10 and selectively move proximal to mu-metal pathway 722 to extend the magnetic field to the magnetic field terminus, the magnetic field terminus is configured to be moved proximate magnetic sensor 354 to extend the magnetic field across drape 46 from sterile space 10 to working space 12, and magnetic sensor 354 is configured to be actuated in working space 12 via the magnetic field extending from the magnetic field terminus to magnetic sensor 354.

In some embodiments, foot support 700 may include a foot support attachment comprising the magnetic field terminus, which may be foot support 700, and the magnetic field may actuate foot support module 350 to grip the foot support attachment via gripping mechanism 352. Foot support 700 may include user interface paddle 710 configured to be moved from a first position in which magnets 706 are not proximal to mu-metal pathway 722 and do not generate the magnetic field at the magnetic field terminus, to a second position in which magnets 706 are proximal to mu-metal pathway 722 and extend the magnetic field to the magnetic field terminus; user interface paddle 710 may be biased to the first position by springs 740 and may include a detent configured to lock user interface paddle 710 in at least one of the first position or a third position outward of the second position. In the second position of user interface paddle 710, distal end 502 of spar 500 may fold and proximal end 504 of spar 500 may translate substantially simultaneously. The magnetic field terminus may include rotational axis 734 about which foot support 700 may rotate in a pitch direction relative to foot support module 350. The system may further include joint 520 disposed between and coupling proximal end 504 and distal end 502 of spar 500, joint 520 being configured to release to flex spar 500 generally in a posterior direction such that an inferior window 116 may be formed proximate spar 500 to allow access to a knee generally from an inferior direction.

A spar 500 for supporting a leg 32 of a patient 20 for a surgical procedure may include a proximal end 504 configured to couple to a surgery table 200; a distal end 502 opposing the proximal end 504; a joint 520 disposed between and configured to couple the proximal end 504 to the distal end 502, the joint 520 further configured to rotate and flex the spar 500 in a generally posterior direction 107 when coupled to the surgery table 200, the joint 520 comprising an angle 503 between the proximal end 504 and the distal end 502 configured to be reduced as the joint 520 is rotated; and a foot support 700 coupled to and disposed at the distal end 502 of the spar 500 and configured to secure a foot 36 of the leg 32 of the patient 20, wherein the spar 500 is configured to flex in the generally posterior direction 107 as the leg 32 is flexed in a generally anterior direction 105 opposing the generally posterior direction 107 such that the leg 32 is able to flex away from the spar 500 and without the spar 500 being directly between an upper leg 33 and a lower leg 35 of the leg 32 when the leg 32 is flexed.

In some embodiments, the joint 520 may be configured to rotate to flex the spar 500 to reduce the angle 503 between the proximal end 504 and the distal end 502 to an acute angle such that an inferior window 116 is formed proximate to the spar 500 to allow access to a knee 34 of the leg 32 generally from an inferior direction 103.

In some embodiments, the joint 520 may be configured to rotate in a plane generally parallel to a plane containing the leg 32 such that the spar 500 and the leg 32 flex in generally parallel planes.

In some embodiments, the joint 520 may be configured to be locked to fix the distal end 502 of the spar 500 relative to the proximal end 504 of the spar 500 at a plurality of angles, the plurality of angles comprising the angle 503.

In some embodiments, the spar 500 may further include a control 562 disposed at the distal end 502 of the spar 500, wherein the joint 520 is configured to be unlocked by the control 562.

In some embodiments, the spar 500 may further include a handle 560 disposed at the distal end 502 of the spar 500, the handle 560 comprising the control 562.

In some embodiments, the spar 500 may comprise a straight spar configuration 506 in which the spar 500 is not pitched or yawed, wherein the straight spar configuration 506 comprises the spar 500 extending generally along a horizontal plane of the surgery table 200 and along an axis 510 generally parallel to a centerline 110 of the surgery table 200, and wherein the spar 500 is configured to yaw from the straight spar configuration 506 inward and outward relative to the centerline 110.

In some embodiments, the spar 500 may be configured to yaw from the straight spar configuration 506 inward about 20 degrees, and the spar 500 may be configured to yaw from the straight spar configuration 506 outward about 40 degrees.

In some embodiments, the spar 500 may be configured to yaw outward such that a medial window 114 and a lateral window 118 are formed proximate to the spar 500 to allow access to the knee 34 of the leg 32 from generally a medial direction 102 and generally a lateral direction 104.

In some embodiments, the joint 520 may be configured to rotate to flex the spar 500 to reduce the angle 503 between the proximal end 504 and the distal end 502 to an acute angle to allow further access to the knee 34 of the leg 32 from a generally inferior direction 103.

In some embodiments, as the spar 500 is flexed, the spar 500 may be configured to translate in a superior direction 101 relative to the surgery table 200 and lower in generally a posterior direction 107 such that a height of a knee 34 of the leg 32 of the patient 20 is generally maintained as the spar 500 is flexed.

A system 100 for supporting a patient 20 for a surgical procedure may include a surgery table 200 comprising a distal end 202, a proximal end 204 opposing the distal end 202, and a platform 220 configured to support at least a portion of the patient 20; a first spar 500 configured to support a first leg 32 of the patient 20, the first spar 500 comprising a proximal end 504 coupled to the surgery table 200, a distal end 502 opposing the proximal end 504 of the first spar 500, a joint 520 disposed between and configured to couple the proximal end 504 to the distal end 502 of the first spar 500, the joint 520 further configured to rotate and flex the first spar 500 in a generally posterior direction 107 when coupled to the surgery table 200, an angle 503 between the proximal end 504 and the distal end 502 configured to be reduced as the joint 520 is rotated, and a foot support 700 coupled to and disposed at the distal end 502 of the first spar 500 and configured to secure a foot 36 of the leg 32 of the patient 20; a second spar 500 configured to support a second leg 32 of the patient 20, the second spar 500 coupled to the surgery table 200; and an operable leg drape portion 72 configured to be disposed over at least a portion of the first leg 32, and a non-operable leg drape portion 74 configured to be disposed over at least a portion of the second leg 32, the operable leg drape portion 72 and the non-operable leg drape portion 74 configured to form a sterile zone 98 between the first leg 32 and the second leg 32, and allow a user 40 to stand between the first leg 32 and the second leg 32, wherein the first spar 500 is configured to flex generally in a posterior direction 107 as the leg 32 is flexed generally in an anterior direction 105 opposing the posterior direction 107 such that the leg 32 is able to flex away from the first spar 500 and without the first spar 500 being directly between an upper leg 33 and a lower leg 35 of the leg 32.

In some embodiments, the operable leg drape portion 72 may comprise a leg opening 68 configured to encircle at least a portion of the first leg 32.

In some embodiments, the system 100 may further include a wall drape portion 73 configured to extend between the operable leg drape portion 72 and the non-operable leg drape portion 74 to form at least a portion of the sterile zone 98, wherein the wall drape portion 73 is configured to extend generally vertically between the operable leg drape portion 72 and the non-operable leg drape portion 74.

In some embodiments, the joint 520 may be configured to rotate to flex the first spar 500 to reduce the angle 503 between the proximal end 504 and the distal end 502 of the first spar 500 to an acute angle such that an inferior window 116 is formed proximate to the first spar 500 to allow access to a knee 34 of the first leg 32 generally from an inferior direction 103.

In some embodiments, the joint 520 may be configured to be locked to fix the distal end 502 of the first spar 500 relative to the proximal end 504 of the first spar 500 at a plurality of angles comprising the angle 503.

In some embodiments, the first spar 500 may comprise a straight spar configuration 506 in which the first spar 500 is not pitched or yawed, wherein the first spar 500 is configured to yaw from the straight spar configuration 506 inward and outward, wherein the first spar 500 extends generally along a horizontal plane of the surgery table 200 and along an axis 510 generally parallel to a centerline 110 of the surgery table 200 in the straight spar configuration 506, and wherein yaw inward and outward of the first spar 500 is relative to the centerline 110.

In some embodiments, the first spar 500 may be configured to yaw outward such that a medial window 114 and a lateral window 118 are formed proximate to the first spar 500 to allow access to the knee 34 of the leg 32 from generally a medial direction 102 and generally a lateral direction 104.

In some embodiments, the second spar 500 may comprise a second straight spar configuration 506 in which the second spar 500 is not pitched or yawed, wherein the second spar 500 is configured to yaw from the second straight spar configuration 506 inward and outward, wherein the second spar 500 extends generally along a horizontal plane of the surgery table 200 and along an axis generally parallel to a centerline 110 of the surgery table 200 in the straight spar configuration 506, wherein yaw inward and outward of the second spar 500 is relative to the centerline 110, and wherein the second spar 500 is configured to yaw outward to form the sterile zone 98 between the first leg 32 and the second leg 32.

In some embodiments, as the first spar 500 is flexed, the first spar 500 may be configured to translate in a superior direction 101 relative to the surgery table 200 and lower in generally a posterior direction 107 such that a height of a knee 34 of the leg 32 of the patient 20 is generally maintained.

It should be noted that the use of particular terminology when describing certain features or embodiments of the disclosure should not be taken to imply that the terminology is being re-defined herein to be restricted to include any specific characteristics of the features or embodiments of the disclosure with which that terminology is associated. Terms and phrases used in this application, and variations thereof, especially in the appended claims, unless otherwise expressly stated, should be construed as open-ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least”; the term “such as” should be interpreted as “such as, without limitation”; the term “includes” should be interpreted as “includes but is not limited to”; the term “example” is used to provide exemplary instances of the item in discussion, not an exhaustive or limiting list thereof, and should be interpreted as “example, but without limitation”; adjectives such as “known,” “normal,” “standard,” and terms of similar meaning should not be construed as limiting the item described to a given time period or to an item available as of a given time, but instead should be read to encompass known, normal, or standard technologies that may be available or known now or at any time in the future; and use of terms like “preferably,” “preferred,” “desired,” or “desirable,” and words of similar meaning should not be understood as implying that certain features are critical, essential, or even important to the structure or function of the present disclosure, but instead as merely intended to highlight alternative or additional features that may or may not be utilized in a particular embodiment.

Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

In addition, as used herein, the term “coupled” means connected to or joined to or placed into communication with, either directly or through intermediate components. The term “patient” is broadly defined herein to include human patients of all sizes, genders and demographics, as well as animals (e.g., for veterinarian purposes). The terminology includes the words noted above, derivatives thereof and words of similar import.

It will be appreciated by those skilled in the art that changes could be made to the embodiments described above without departing from the broad inventive concept thereof. For example, various mechanical and electrical connection elements and actuators may be used to achieve the disclosed function. It is understood, therefore, that this invention is not limited to the particular embodiments disclosed, but it is intended to cover modifications within the spirit and scope of the present invention as defined by the appended claims.

What is claimed is:

1. A spar for supporting a leg of a patient for a surgical procedure, the spar comprising:
a proximal end configured to couple to a surgery table;
a distal end opposing the proximal end;
a joint disposed between and configured to couple the proximal end to the distal end, the joint further configured to rotate and flex the spar in a generally posterior direction when coupled to the surgery table, the joint comprising an angle between the proximal end and the distal end configured to be reduced as the joint is rotated; and
a foot support coupled to and disposed at the distal end of the spar and configured to secure a foot of the leg of the patient,
wherein the spar is configured to flex in the generally posterior direction as the leg is flexed in a generally anterior direction opposing the generally posterior direction such that the leg is able to flex away from the spar and without the spar being directly between an upper leg and a lower leg of the leg when the leg is flexed.

2. The spar of claim 1, wherein the joint is configured to rotate to flex the spar to reduce the angle between the proximal end and the distal end to an acute angle such that an inferior window is formed proximate to the spar to allow access to a knee of the leg generally from an inferior direction.

3. The spar of claim 1, wherein the joint is configured to rotate in a plane generally parallel to a plane containing the leg such that the spar and the leg flex in generally parallel planes.

4. The spar of claim 1, wherein the joint is configured to be locked to fix the distal end of the spar relative to the proximal end of the spar at a plurality of angles, the plurality of angles comprising the angle.

5. The spar of claim 4, further comprising:
a control disposed at the distal end of the spar,
wherein the joint is configured to be unlocked by the control.

6. The spar of claim 5, further comprising:
a handle disposed at the distal end of the spar,
the handle comprising the control.

7. The spar of claim 1, wherein the spar comprises a straight spar configuration in which the spar is not pitched or yawed,
wherein the straight spar configuration comprises the spar extending generally along a horizontal plane of the surgical table and along an axis generally parallel to a centerline of the surgical table, and
wherein the spar is configured to yaw from the straight spar configuration inward and outward relative to the centerline.

8. The spar of claim 7, wherein the spar is configured to yaw from the straight spar configuration inward about 20 degrees, and
wherein the spar is configured to yaw from the straight spar configuration outward about 40 degrees.

9. The spar of claim 7, wherein the spar is configured to yaw outward such that a medial window and a lateral window are formed proximate to the spar to allow access to the knee of the leg from generally a medial direction and generally a lateral direction.

10. The spar of claim 9, wherein the joint is configured to rotate to flex the spar to reduce the angle between the proximal end and the distal end to an acute angle to allow further access to the knee of the leg from a generally inferior direction.

11. The spar of claim 1, wherein, as the spar is flexed, the spar is configured to translate in a superior direction relative to the surgery table and lower in generally a posterior direction such that a height of a knee of the leg of the patient is generally maintained as the spar is flexed.

12. A system for supporting a patient for a surgical procedure, the system comprising:
a surgery table comprising a distal end, a proximal end opposing the distal end, and a platform configured to support at least a portion of the patient;
a first spar configured to support a first leg of the patient, the first spar comprising:
a proximal end coupled to the surgery table,
a distal end opposing the proximal end of the first spar,
a joint disposed between and configured to couple the proximal end to the distal end of the first spar, the joint further configured to rotate and flex the spar in a generally posterior direction when coupled to the surgery table, an angle between the proximal end and the distal end configured to be reduced as the joint is rotated, and
a foot support coupled to and disposed at the distal end of the first spar and configured to secure a foot of the leg of the patient;
a second spar configured to support a second leg of the patient, the second spar coupled to the surgery table; and
an operable leg drape portion configured to be disposed over at least a portion of the first leg,
a non-operable leg drape portion configured to be disposed over at least a portion of the second leg, the operable leg drape and the non-operable leg drape configured to form a sterile zone between the first leg and the second leg, and allow a user to stand between the first leg and the second leg,
wherein the first spar is configured to flex generally in a posterior direction as the leg is flexed generally in an anterior direction opposing the posterior direction such that the leg is able to flex away from the spar and without the spar being directly between an upper leg and a lower leg of the leg.

13. The system of claim 12, wherein the operable leg drape portion comprises a leg opening configured to encircle at least a portion of the first leg.

14. The system of claim 12, further comprising:
a wall drape portion configured to extend between the operable leg portion and the non-operable leg portion to form at least a portion of the sterile zone,
wherein the wall drape portion is configured to extend generally vertically between the operable leg portion and the non-operable leg portion.

15. The system of claim 12, wherein the joint is configured to rotate to flex the first spar to reduce the angle between the proximal end and the distal end of the first spar to an acute angle such that an inferior window is formed proximate to the first spar to allow access to a knee of the first leg generally from an inferior direction.

16. The system of claim 12, wherein the joint is configured to be locked to fix the distal end of the first spar relative to the proximal end of the first spar at a plurality of angles comprising the angle.

17. The system of claim 12, wherein the first spar comprises a straight spar configuration in which the first spar is not pitched or yawed,
wherein the first spar is configured to yaw from the straight spar configuration inward and outward,
wherein the first spar extends generally along a horizontal plane of the surgical table and along an axis generally parallel to a centerline of the surgical table in the straight spar configuration, and
wherein yaw inward and outward of the first spar is relative to the centerline.

18. The system of claim 17, wherein the first spar is configured to yaw outward such that a medial window and a lateral window are formed proximate to the first spar to allow access to the knee of the leg from generally a medial direction and generally a lateral direction.

19. The system of claim 18, wherein the second spar comprises a second straight spar configuration in which the second spar is not pitched or yawed,
wherein the second spar is configured to yaw from the second straight spar configuration inward and outward,
wherein the second spar extends generally along a horizontal plane of the surgical table and along an axis generally parallel to a centerline of the surgical table in the straight spar configuration,
wherein yaw inward and outward of the second spar is relative to the centerline, and
wherein the second spar is configured to yaw outward to form the sterile zone between the first leg and the second leg.

20. The system of claim 12, wherein, as the first spar is flexed, the first spar is configured to translate in a superior direction relative to the surgery table and lower in generally a posterior direction such that a height of a knee of leg of the patient is generally maintained.
